(19) United States
(12) Patent Application Publication
(10) Pub. No.: US 2022/0148699 A1
(43) Pub. Date: May 12, 2022

(54) SYSTEMS AND METHODS FOR HOSTING WELLNESS PROGRAMS

(71) Applicant: YourCoach Health, Inc., Brooklyn, NY (US)

(72) Inventors: Daniel Kogan, Brooklyn, NY (US); Marina Borukhovich, Barcelona (ES); Eugene Borukhovich, Barcelona (ES); Ilya Sivkov, Omsk (RU)

(21) Appl. No.: 17/520,534

(22) Filed: Nov. 5, 2021

Related U.S. Application Data
(60) Provisional application No. 63/111,052, filed on Nov. 8, 2020.

(51) Int. Cl. G16H 20/30; G16H 10/60

(52) U.S. Cl. CPC G16H 20/30; G16H 10/60

(57) ABSTRACT

Systems and methods for matching a client and a coach are provided. A request for a wellness program including a set of attributes is received. A plurality of coaching profiles is obtained, each associated with a corresponding coach and includes a corresponding one or more wellness programs and a first corresponding data set associated with performance of the corresponding coach. Responsive to the request, a plurality of wellness programs is obtained, each associated with one or more corresponding coaches and includes one or more attributes improved by the respective wellness program and a second corresponding data set associated with performance of the respective wellness program. The coaching profiles, the wellness programs, and the set of attributes are processed, producing a respective result for each computational model that is collectively considered, producing a set of at least one coaching profile and wellness program that is communicated to a remote device.

### CROSS-REFERENCE TO RELATED APPLICATION

The present application claims priority to U.S. Provisional Application No. 63/111,052, entitled “Machine Learning System for Operating on Data of an Online Wellness Platform,” filed Nov. 8, 2020, which is hereby incorporated by reference herein.

### TECHNICAL FIELD

The present disclosure relates to systems and methods for hosting wellness programs. More particularly, the systems and methods of the present disclosure relate to matching a client with a coach.

### BACKGROUND

Recently, industries have turned to computer implemented matching programs to coordinate the assignment of individuals to practicing entities.

For instance, the National Resident Matching Program (NRMP) seeks to optimize matching of applications into a resident program. The NRMP collects information from applicants that includes examination scores of an applicant, membership of the applicant in a fraternal honor society, research experience of the applicant, and educational experience of the applicant. See Rinard et al., 2010, “Successfully Matching into Surgical Specialties: An Analysis of National Resident Matching Program Data,” Journal of Graduate Medical Education, 2(3), pg. 316. From this, a student performance evaluation is provided to resident program directors to evaluate relative scientific and professional attributes of each applicant. Even though these evaluations are useful for considering each applicant with respect to an inherent variability in grading across educational institutions, the evaluations are written using inconsistent methods. Furthermore, these evaluations lend themselves to inconsistent interpretation, which augments prior inconsistences in the evaluations. To remedy this, conventional systems seek to normalizing unusual characteristics by removing inconsistencies and lack of objectivity such as emphases, strengths, missions, and goals of an applicant or educational entity. See Boysen-Osborn et al., 2017, “Who to Interview? Low Adherence by U.S. Medical Schools to Medical Student Performance Evaluation Format Makes Resident Selection Difficult,” Western Journal of Emergency Medicine, 18(1), pg. 50. In this way, conventional solutions lack a mechanism to obtain either direct or indirect feedback from the applicant during the evaluation process.

Moreover, online health and wellness coaching has grown rapidly. For instance, recently many platforms have been created to assist coaches in content hosting, communicating, billing and invoice systems, and other related activities such as calendaring appointments. In the field of coaching large organizations such as an office of employees, numerous online and mobile tools exist. These tools include hybrids of contractual agreements to be signed by parties, billing and invoices system, overall program content, program schedulers, messenger with video conferencing capabilities, and content management system. Even though conventional systems have capabilities such as dynamic capacity management, search features, matching and recommendation systems, notifications, notes, journal features, analytics to measure the sentiment and/or user engagement. However, none of these conventional solutions exist as a complete, effective, efficient, online wellness platform. For instance, conventional systems are typically disparate third-party tools stitched together to support custom workflows implemented for a company that uses them. The result is these solutions cannot be used as a product that others can re-use. Those platforms that truly support the marketplace are focused on directory-like listings with basic searches and do not address the tools that support individual contributors on the platform.

One such example of a conventional system is a content hosting platform. This conventional solutional contains courses for many topics related to health and wellness and provides a wide range of functionality for both coaches and clients. Also, more topic-specific platforms exist for particular disciplines such as yoga or nutrition. These conventional platforms have a search for content based on keywords. Moreover, some conventional solutions include a recommendation engine based on keywords or demand. In both cases when a user chooses content he or she relies on their own experience or on the recommendations or opinions of other users. However, with all the prior attempts, no medical or scientific background is provided to guide a user on what course to choose. For users, this leads to choosing courses that end up with frustrating or non-relevant results. For the service provider, this leads to skewed statistics (e.g., popular courses become even more popular just because they are presented first), noisy data, and makes performance evaluation of coaches and clients very difficult. Furthermore, these conventional platforms do not incorporate feedback from the client for the coach and the coach. Moreover, the conventional platforms do not provide oversight when the coach performs a course with the client or generally engages with the platform.

As such, there is a need for systems and methods for improving how a user is onboarded to provide and/or access coaching, matching the user with a coaching program, providing oversight when conducting the coaching program, and incorporating feedback for coach and programs, or a combination thereof.

### SUMMARY

Given the above background, what is needed in the art are systems and methods that encourage and improve engagement amongst clients and coaches, either collectively or individually, with a wellness system. In some embodiments, this improved engagement is facilitated by: managing each client, each coach, and each wellness program provided by a coach; hosting each wellness program; hosting profiles and forums for promoting collaborations; conducting and performing each wellness program; monitoring the conducting and performance of each wellness program; obtaining one or more data sets associated with a respective coach and/or a respective client during the monitoring of each wellness program; improving each wellness program and abilities of the coach; or a combination thereof.

In some embodiments, by improving engagement using the systems and methods of the present disclosure, a respective coach and/or a respective client has higher levels of digital activities. From this, the systems and methods of the present disclosure obtain richer data sets and information associated with the respective coach and/or the respective client in the form of historical data sets and/or corpus of communications. The historical data sets include a corresponding historical performance of a corresponding coach, such as a quality of the respective coach during a respective wellness program, a quality of the respective wellness program (e.g., based on feedback provided from different clients that are deemed to have completed the respective wellness program), a popularity of the respective coach, a popularity of the respective wellness program, or a combination thereof. However, the present disclosure is not limited thereto. In some embodiments, the historical data sets include one or more goals associated with a respective client of the respective coach, an accuracy and/or precision of the respective wellness program, a return of investment of the respective client, or a combination thereof. Moreover, in some embodiments, the one or more data sets in the form of the corpus of communications is obtained from open, public resources, such as from scholarly research articles and empirical processes. With these data sets, the systems and methods of the present disclosure utilize a plurality of computational models to, in turn, determine resultant data sets required to match a client with a best coach, such as confidence and quality scores of a respective coach. As another non-limiting example, in some embodiments, with these data sets, the systems and methods of the present disclosure utilize a plurality of computational models to determine one or more recommendations for improving engagement with a respective wellness program associated with the respective coach. However, the present disclosure is not limited thereto. Accordingly, in some such embodiments, the systems and methods of the present disclosure obtain information from external sources, such as one or more corpus of communications associated from a scholarly publication.

Accordingly, in some embodiments, the present disclosure provides systems and methods for hosting a health and wellness coaching platform that provides one or more recommendations for the coach, such as when crafting wellness programs and/or engaging (a plurality of clients and a plurality of coaches) associated with the health and wellness coaching platform (e.g., a wellness system). In some embodiments, the one or more recommendations is provided to a respective coach when creating a wellness program, editing the wellness program, conducting the wellness program with a client, or a combination thereof.

As such, in some embodiments, the wellness system allows for onboarding the end-users by receiving a set of attributes attributed to a respective end user and providing one or more recommendations based on the set of attributes. In some embodiments, the systems and methods of the present disclosure facilitates onboarding the plurality of clients by providing a survey to a respective client and/or receiving a response to the survey from the respective client. In some such embodiments, the response to the survey is in the form of a request for a wellness program with the set of attributes assigned to the respective client. Non-limiting examples of one or more attributes in the set of attributes assigned to the respective client include one or more keywords associated with a description of a wellness program, a focus (e.g., discipline) of the wellness program, one or more conditions exhibited or experienced by the respective client (e.g., medical conditions), one or more user profile attributes (e.g., geographic region, age, wealth, expertise, willingness to pay, gender, profession, lifestyle, etc.), or a combination thereof. In some embodiments, the respective client and/or the wellness system (e.g., using one or more computational models in a plurality of computational models) assigns or suggests a weight for each respective attribute in the set of attributes.

In some embodiments, the systems and methods of the present disclosure facilitate onboarding the plurality of coaches by facilitating a process for a respective coach to create and/or edit a wellness program that is hosted and accessible through the wellness system (e.g., by a graphical user interface of a client application presented through a display of a client device). In some embodiments, the process for allowing the respective coach to create the wellness program includes providing one or more prompts to the respective coach through a graphical user interface of a client application that is executed on a remote client device associated with the respective coach. The one or more prompts is configured to receive a set of attributes that are to be assigned to the respective coach. In some embodiments, the set of attributes includes the wellness program title, a description of the wellness program, a discipline of the wellness program, one or more conditions associated with the wellness program (e.g., one or more goals), or a combination thereof. Accordingly, in some such embodiments, the systems and methods of the present disclosure provide a revised set of attributes that is considered, by a plurality of computational models, to be a best set of attributes for the wellness program. In some embodiments, the respective coach and/or the wellness system (e.g., using one or more computational models in a plurality of computational models) assigns a weight for each respective attribute in the set of attributes. For instance, in some such embodiments, each respective attribute in the set of attributes is assigned an independent weight by the respective coach or the wellness system. This revised set of attributes includes none, some, or all of the one or more attributes in the set of attributes assigned to the respective coach.

In some embodiments, the systems and methods of the present disclosure utilize one or more data sets from open, public resources, such as one or more corpus of communications obtained from scholarly research articles, empirical processes, and/or one or more historical data sets obtained when the plurality of clients and/or the plurality of coaches engage with the wellness system during usage. From this, in some embodiments, the systems and methods of the present disclosure provide wellness guidance, such as one or more recommendations for improving performance of the respective wellness program for one or more prospective clients. In some embodiments, the systems and methods of the present disclosure provides guidance by determining if a positive sentiment is associated with the respective coach when communicating with a respective client in a communication channel of the wellness system. In some embodiments, the systems and methods of the present disclosure provides guidance by determining if a non-confrontational manner is associated with the respective coach when communicating with clients in a communication channel associated with or used by the wellness system. This communication channel can be, for example, E-mail, a blog, text messages, or an electronic conversation. In some embodiments, the systems and methods of the present disclosure provide guidance to the respective coach when creating a wellness program, editing the wellness program, conducting the wellness program, mentoring a client (e.g., mentoring an apprentice coach), or a combination thereof. In some embodiments, the one or more recommendations is provided to a respective client when assigning a set of attributes to the respective client and/or suggesting the wellness program for the respective client.

Moreover, in some embodiments, the systems and methods of the present disclosure provide matching the respective client with one or more coaches and/or one or more wellness programs using a plurality of computational models. Each computational model in the plurality of computational models is configured to produce a respective result that is a data set. For instance, in some embodiments, the respective result is based on a similarity of two or more user profiles (e.g., to determine a respective match for one or more wellness programs deemed complete by similar clients). In some embodiments, the respective result is based on a similarity of at least two sets of attributes, such as two sets of goals (e.g., to determine a respective match for one or more wellness programs with one or more attribute similar to the at least to sets of attributes). In some embodiments, the respective result is based on a similarity of at least two sets of attributes and at least two sets of wellness programs (e.g., to determine a third recommendation for one or more wellness programs that satisfy each attribute in a set of attributes assigned to the client). In some embodiments, the respective result is based on a similarity of least two texts in one or more corpus of communications (e.g., to determine a fourth recommendation for one or more clusters of a plurality of wellness programs, a plurality of communication channels, a plurality of material, or a combination there). In some embodiments, the respective result is based on a similarity of at least two wellness programs (e.g., to determine a recommendation if a respective wellness program has an incorrect attribute in a set of attributes assigned to a respective coach). However, the present disclosure is not limited thereto.

In some embodiments, the systems and methods of the present disclosure provide one or more computer system implemented tools through a client application executed at a remote client device. In some embodiments, the client application allows experienced coaches to mentor less experienced coaches (e.g., a respective client that is an apprentice coach). For instance, in some embodiments, the systems and methods of the present disclosure allow a first coach that is experienced, or vetted, in a first discipline (e.g., licensed tennis professional) to be matched with a second coach that is unexperienced in the first disciplined (e.g., unlicensed tennis professional). From this matching of the first coach with the second coach, in some embodiments, a first coaching team is formed that conducts one or more wellness programs.

In some embodiments, the systems and methods of the present disclosure enable the respective coach to lend their expertise to the second coach or the respective client through one or more wellness programs, one or more publications, one or more surveys, or a combination thereof that is authored, attributed, or administrated by the respective coach through a wellness system.

In some embodiments, the systems and methods of the present disclosure facilitate obtaining one or more data sets in order to develop one or more evidence-based and/or scientifically-backed wellness programs. In some embodiments, the one or more wellness programs developed by the systems and methods of the present disclosure is utilized by one or more coaches (e.g., as a foundation for creating different wellness programs) and one or more clients (e.g., to perform with a coach). In some embodiments, this obtaining of the one or more data sets is conducted when the respective client and/or the respective coach is engaging with the wellness system, such as by communicating in a communication channel (e.g., publishing one or more messages) of the wellness system or when conducting the one or more wellness programs. However, the present disclosure is not limited thereto.

More particularly, the systems and methods of the present disclosure at least provide one or more computer implemented wellness programs that is facilitated by coaching for one or more clients provided by one or more coaches. Moreover, the one or more wellness programs is supported by computational analysis performed by a plurality of computational models. In this way, the computational analysis performed by the plurality of computational models is based, at least in part, on one or more historical data sets obtained by the systems and methods of the present disclosure as well as public data set sources, which ensures scientific accuracy and precision when evaluating a respective coach and/or a respective wellness program (e.g., for matching with a first client). A non-limiting example of this support provided by the plurality of computational models includes providing one or more recommendations to the respective coach when creating and/or editing the one or more wellness programs. Yet another non-limiting example of this support includes providing one or more recommendations to the respective coach responsive to the respective coach communicating with the one or more clients and/or conducting the one or more wellness programs (e.g., based on a corresponding historical data set associated with the respective coach and/or the one or more wellness programs).

In some embodiments, the systems and methods of the present disclosure include the plurality of computational models that provide the plurality of clients and the plurality of coaches with a high-quality wellness system for creation of the one or more wellness programs, evaluation of such one or more wellness programs, commercialization of the one or more wellness programs, or a combination thereof. In some embodiments, the plurality of computational models is configured to promote engagement with the wellness system. In some embodiments, the plurality of computational models that provide the plurality of clients and the plurality of coaches with one or more quality assessment metrics. In some embodiments, the one or more quality assessment metrics is application for a respective coach and/or a respective wellness program.

In some embodiments, each computational model in the plurality of computational models generates a result that is a data set, such as a respective historical data set of one or more analytics and key performance indicator (KPI) measurements. The results are collectively considered from the plurality of computational models to determine one or more recommendations, such as a best performing coach, a first best fit between a coach and a client, a second best fit between the client and a wellness program, or a combination thereof. In some embodiments, the systems and methods of the present disclosure provide coaches with an incentive to use the wellness platform and generate data sets, in order to not only provide a robust collection of information for use by the wellness system when providing such one or more recommendations, but also to improve the skills of the coaches as the coaches engage with wellness system. In this way, the historical data sets of the systems and methods of the present disclosure provide a feedback mechanism to give coaches an incentive to engage with the wellness system and generate such data sets for analytics and assessment by the plurality of computational models.

In some embodiments, the systems and methods of the present disclosure use acquired and/or determined data sets to govern one or more aspects of the present disclosure including one or more recommendations for a coach and/or a wellness program, one or more reputation management suggestions for the coach or a client, one or more return on investment (ROI) evaluations, or any combination thereof.

In some embodiments, the systems and methods of the present disclosure suggest and/or modify one or more attributes associated with a corresponding wellness program associated with the coach. An example of this is modifying a first attribute that is a title of the wellness program, a second attribute that is a description of the wellness program, a third attribute that is a focus of the wellness program, a fourth attribute that is a length (duration) of the wellness program, a fifth attribute that is the price of the wellness program, or the like.

In some embodiments, the systems and methods of the present disclosure provide adaptive analytics in the form of a respective result by a computational model that incorporates feedback from one or more subjects, such as from one or more coaches, one or more clients, and one or more entities (e.g., corporate entity).

In some embodiment, the systems and methods of the present disclosure allow for a gamification of the wellness system. In some embodiments, the gamification encourages a user to engage with the wellness system, such as participate in a communication channel, create a wellness program, or conduct the wellness program. As a non-limiting example, in order to accumulate a robust pool of information from the user when in a respective wellness program. This gamification of the wellness program allows a client and/or a coach to consistently, or constantly, measure a level of quality of one or more wellness programs and/or coaches. Furthermore, the systems and methods of the present disclosure incentivizes the coach to spend more time engaging with the systems and methods of the present disclosure. This gamification produces more content and data resulting in improved analytics and ultimately better matches and recommendations of a set of one or more coaches and one or more wellness programs for the user. Furthermore, in some embodiments, the gamification of the wellness programs challenges the coach to improve coaching skills and wellness program content.

In some embodiments, the systems and methods of the present disclosure provide a recommendation of a set of one or more coaches and one or more wellness programs. The systems and methods evaluate data sets associated with a respective client, a respective coach, a respective wellness program, or a combination thereof. In some embodiments, the recommendation provides a prediction of a utility of using the set of one or more coaches and the one or more wellness programs.

In some embodiments, a client is provided with a computational-based evaluation and historical data set recommendation of the set of one or more coaches and/or one or more wellness programs based on one or more attributes assigned to the client. In some embodiments, the recommendation is generated by a multi-disciplinary approach to the wellbeing and treatment of health concerns of the client. For instance, in some embodiments, one or more connections is determined between an attribute of a wellness program and a related topic that is effective when addressed together, between a depth of the wellness program description, relevance of the description of the wellness program, or relevance of the wellness program content, in order to optimize the wellness program for success by a respective client.

In some embodiments, one or more attributes is connected to one or more medical conditions and/or treatments via scientific publications. In some embodiments this is done in order to measure (determine) efficacy, accuracy, precision, or a combination thereof of such treatments when used with a respective wellness program based on the nature of such research and clinical trials. Said otherwise, in some embodiments, the wellness system utilizes the scientific foundation based on some quantification of accuracy and/or precision provided by a respective wellness program in comparison against one or more corpus of communications associated with scientific publications and vetted sources (e.g., a government agency entity). A non-limiting example of the one or more attributes that is connected to the one or more medical conditions and/or treatments includes subject lifestyle, the subject's personal development goals, the subject's mental health, a medical condition of the subject, or a combination thereof. From this, in some such embodiments, the wellness system provides the respective coach with a recommendation of a revised set of attributes based on a consideration of a respective attribute in a set of attributes that is assigned to the respective coach. In such embodiments, the respective attribute is considered against one or more attributes that is associated with one or more medical conditions and/or treatments associated with the scientific publications or vetted sources, such as clinical trial for a hypertension attribute.

In some embodiments, the systems and methods of the present disclosure provide onboarding for a coach. In some embodiments, this onboarding for the coach includes management and retaining features enabled via gamification where coaches are constantly challenged to engage with the systems and methods of the present disclosure. From this engagement with the systems and methods of the present disclosure, the coaches further improve their skills at conducting wellness programs with clients, recruiting clients for wellness programs, content of the wellness programs, or a combination thereof by the means of utilizing recommendations and matching the clients with the coaches.

Turning to other specific aspects of the present disclosure, one aspect of the present disclosure is directed to providing a method of matching a first client and a coach. The method is performed at a computer system. The computer system includes one or more processors and a memory storing at least one program for execution by the at least one processor. The at least one program includes instructions for receiving, in electronic form, a request for a wellness program. The request includes a set of attributes assigned to the first client from a plurality of attributes. Moreover, the at least one program includes instructions for obtaining a plurality of coaching profiles in response to receiving the request. Each coaching profile in the plurality of coaching profiles is associated with a corresponding coach. Furthermore, each coaching profile includes a corresponding one or more wellness programs. Each wellness program in the corresponding one or more wellness programs is administered, at least in part, by the corresponding coach. Additionally, each coaching profile includes a first corresponding data set that is associated with a corresponding first historical performance of the corresponding coach during a respective wellness program in the corresponding one or more wellness program. The at least one program further includes instructions for further obtaining a plurality of wellness programs in response to receiving the request. Each respective wellness program in the plurality of wellness program is associated with one or more corresponding coaches in the plurality of coaches. Furthermore, each respective wellness program includes one or more attributes improved by the respective wellness program. Additionally, each respective wellness program includes a second corresponding data set associated with a second historical performance of the respective wellness program during the respective wellness program. The at least one program includes instructions for processing the plurality of coaching profiles, the plurality of wellness programs, and the set of attributes assigned to the first client using a plurality of computational models. From this, the at least one program includes instructions for producing a respective result for each computational model in the plurality of computational models. Each respective result is a data set associated with the one or more wellness programs. The at least one program further includes instructions for collectively considering each respective result, which produces a set of at least one coaching profile and at least one wellness program. Accordingly, the at least one program includes instructions for communicating, in electronic format, to a remote device associated with the first client, the set of the at least one coaching profile and the at least one wellness program, which matches the first client and the coach.

In some embodiments, the set of attributes includes one or more medical attributes associated with the first client, one or more temporal attributes associated with completing the respective wellness program, one or more geographic attributes associated with completing the respective wellness program, one or more accounting attributes associated with the respective wellness program, one or more physical attributes associated with the respective wellness program, one or more mental attributes associated with the respective wellness program, or a combination thereof.

In some embodiments, the set of attributes includes the one or more accounting attributes. Moreover, the one or more accounting attributes includes a price of the respective wellness program, a schedule of the respective wellness program, a plurality of tasks associated with the respective wellness program, one or more communication conferences associated with the respective wellness program, one or more quantitative goals associated with the respective wellness program, or a combination thereof.

In some embodiments, the corresponding first historical data set for a corresponding coach in the plurality of coaching profiles includes a universal success rate for the one or more wellness programs associated with the corresponding coach, an individual success rate for each wellness program in the corresponding one or more wellness programs, a universal enrollment rate for the corresponding one or more wellness programs, an individual enrollment rate for each wellness program in the corresponding one or more wellness programs, a communications corpus associated with the corresponding coach, an engagement rate for each wellness program in the corresponding one or more wellness programs, a frequency rate for each wellness program in the corresponding one or more wellness programs, the data set associated with the corresponding one or more wellness programs, or a combination thereof.

In some embodiments, the corresponding first historical data set includes the communications corpus associated with the corresponding coach. The communications corpus includes a record of a corresponding plurality of messages for each communication channel in a plurality of communication channels associated with the corresponding coach.

In some embodiments, each communication channel in the plurality of communication channels facilitates an exchange of a plurality of messages between the corresponding coach and a respective subject in a plurality of subjects.

In some embodiments, the communications corpus provides a temporal ordering to each message in the record of the corresponding exchange of the plurality of messages.

In some embodiments, a respective computational model in the plurality of computational models includes determining, for each respective sentiment in a plurality of sentiments, whether a corresponding sentiment analysis criterion is satisfied or not satisfied by taking a cosine similarly measure or dot product of one or more data elements in the corresponding coaching profile against each reference statement in a corresponding list of reference statements for the respective sentiment that are deemed to be attributive of a predetermined sentiment.

In some embodiments, the corresponding first client is associated with an enterprise that has vetted the coach.

In some embodiments, the at least one program further include instructions for matching the first coach with the corresponding first client, in the corresponding plurality of clients in accordance with an identification, by the plurality of computational models, that the first coach is a respective coach that best matches with a respective attribute of the corresponding first client responsive to receiving a request for a match with the first coach in the set of the at least one coaching profile by the corresponding first client.

In some embodiments, the plurality of computational models includes one or more correlation models, one or more comparison models, one or more regression models, one or more classification models, one or more survival analysis models, one or more product limit estimation models, one or more ranking models, one or more cox proportional hazard models, or a combination thereof.

In some embodiments, the plurality of computational models includes one or more random forest models, one or more random survival forest models, one or more extreme gradient boosting models, one or more support vector machine models, one or more Gaussian mixture models, one or more neural network models, or a combination thereof.

In some embodiments, the data set associated with the one or more wellness programs includes a weighted average of a subset of attributes in the set of attributes assigned to the corresponding first client.

In some embodiments, the data set associated with the one or more wellness program includes a first return of investment of the first client and/or a second return on investment of a respective coach associated with a coaching profile in the set of coaching profiles.

In some embodiments, the communicating further includes generating a listing of the set of the at least one coaching profile and the at least one wellness program for display at the remote device.

In some embodiments, the at least one coaching profile and the at least one wellness program have a one-to-one relationship in the set. In some embodiments, the at least one coaching profile and the at least one wellness program have a one-to-many relationship in the set.

In some embodiments, the respective wellness program is a recreational activity and/or a sport activity.

In some embodiments, the remote device is associated with a subject other than the first client.

In some embodiments, the respective result produced includes a first similarity based on two or more user profiles, a second result based on at least two sets of attributes, a third result based on at least two sets of attributes and at least two sets of wellness programs a fourth result based on at least two texts in one or more corpus of communications, a fifth result based on at least two wellness programs, or a combination thereof.

In some embodiments, the first corresponding data set includes a quality of the respective coach, a quality of a respective wellness program in the one or more wellness programs, a popularity of the respective coach, a popularity of the respective wellness program, or a combination thereof.

In some embodiments, the set of attributes includes a first subset of attributes assigned to the first client by the plurality of computational models and a second subset of attributes assigned to the first client by a human subject.

Another aspect of the present disclosure is directed to providing a computer system for matching a first client and a coach. The computer system includes one or more processors and a memory storing at least one program for execution by the at least one processor. The at least one program includes instructions for receiving, in electronic form, a request for a wellness program. The request includes a set of attributes assigned to the first client from a plurality of attributes. Moreover, the at least one program includes instructions for obtaining a plurality of coaching profiles in response to receiving the request. Each coaching profile in the plurality of coaching profiles is associated with a corresponding coach. Furthermore, each coaching profile includes a corresponding one or more wellness programs. Each wellness program in the corresponding one or more wellness programs is administered, at least in part, by the corresponding coach. Additionally, each coaching profile includes a first corresponding data set that is associated with a corresponding first historical performance of the corresponding coach during a respective wellness program in the corresponding one or more wellness program. The at least one program further includes instructions for further obtaining a plurality of wellness programs in response to receiving the request. Each respective wellness program in the plurality of wellness program is associated with one or more corresponding coaches in the plurality of coaches. Furthermore, each respective wellness program includes one or more attributes that have been improved by the respective wellness program. Additionally, each respective wellness program includes a second corresponding data set associated with a second historical performance of the respective wellness program during the respective wellness program. The at least one program includes instructions for processing the plurality of coaching profiles, the plurality of wellness programs, and the set of attributes assigned to the first client using a plurality of computational models. From this, the at least one program includes instructions for producing a respective result for each computational model in the plurality of computational models. Each respective result is a data set associated with the one or more wellness programs. The at least one program further includes instructions for collectively considering each respective result, which produces a set of at least one coaching profile and at least one wellness program. Accordingly, the at least one program includes instructions for communicating, in electronic format, to a remote device associated with the first client, the set of the at least one coaching profile and the at least one wellness program, which matches the first client and the coach.

Yet another aspect of the present disclosure is directed to providing a non-transitory computer readable storage medium that includes at least one program. The at least one program includes instructions for receiving, in electronic form, a request for a wellness program. The request includes a set of attributes assigned to the first client from a plurality of attributes. Moreover, the at least one program includes instructions for obtaining a plurality of coaching profiles in response to receiving the request. Each coaching profile in the plurality of coaching profiles is associated with a corresponding coach. Furthermore, each coaching profile includes a corresponding one or more wellness programs. Each wellness program in the corresponding one or more wellness programs is administered, at least in part, by the corresponding coach. Additionally, each coaching profile includes a first corresponding data set that is associated with a corresponding first historical performance of the corresponding coach during a respective wellness program in the corresponding one or more wellness program. The at least one program further includes instructions for further obtaining a plurality of wellness programs in response to receiving the request. Each respective wellness program in the plurality of wellness programs is associated with one or more corresponding coaches in the plurality of coaches. Furthermore, each respective wellness program includes one or more attributes improved by the respective wellness program. Additionally, each respective wellness program includes a second corresponding data set associated with a second historical performance of the respective wellness program. The at least one program includes instructions for processing the plurality of coaching profiles, the plurality of wellness programs, and the set of attributes assigned to the first client using a plurality of computational models. From this, the at least one program includes instructions for producing a respective result for each computational model in the plurality of computational models. Each respective result is a data set associated with the one or more wellness programs. The at least one program further includes instructions for collectively considering each respective result, which produces a set of at least one coaching profile and at least one wellness program. Accordingly, the at least one program includes instructions for communicating, in electronic format, to a remote device associated with the first client, the set of the at least one coaching profile and the at least one wellness program, which matches the first client and the coach.

### BRIEF DESCRIPTION OF THE DRAWINGS

FIG. 1 illustrates a block diagram illustrating an embodiment of a system for hosting a plurality of wellness programs, in accordance with an embodiment of the present disclosure;

FIGS. 2A and 2B collectively illustrate a wellness host system for hosting a plurality of wellness programs, in accordance with an embodiment of the present disclosure;

FIG. 3 illustrates a client device for engaging with a respective wellness program, in accordance with an embodiment of the present disclosure;

FIGS. 4A, 4B, 4C, 4D, and 4E collectively provide a flow chart illustrating exemplary methods for hosting a plurality of wellness programs, in which dashed boxes indicate optional features, in accordance with some embodiments of the present disclosure;

FIG. 5 illustrates a user interface for presenting a menu that allows a user to engage with a respective wellness program, in accordance with an embodiment of the present disclosure;

FIG. 6 illustrates a user interface for presenting an evaluation of a description of a wellness program including a revised set of attributes based on a set of attributes assigned to a client, in accordance with an embodiment of the present disclosure;

FIG. 7 illustrates another user interface for presenting an evaluation of a wellness program including a chart of one or more data sets associated with the wellness program, in accordance with an embodiment of the present disclosure;

FIG. 8 illustrates yet another user interface for presenting an evaluation of a wellness program created by a coach, in accordance with an embodiment of the present disclosure;

FIG. 9 illustrates a user interface for matching a client with one or more coaches based on a set of attributes assigned to the client, in accordance with an embodiment of the present disclosure;

FIG. 10 illustrates a user interface for monitoring a communication channel and obtaining a corpus of communications associated with a coach and/or a client, in accordance with an embodiment of the present disclosure;

FIG. 11 illustrates another user interface for monitoring a communication channel and obtaining a corpus of communications associated with a coach and/or a client, in accordance with an embodiment of the present disclosure;

FIG. 12 illustrates a user interface for presenting one or more results provided by a plurality of computational models based on an evaluation of a coach, in accordance with an embodiment of the present disclosure;

FIG. 13 illustrates a flow chart of a workflow for matching a client and a coach, in accordance with an embodiment of the present disclosure;

FIG. 14 illustrates a flow chart of a workflow for obtaining a plurality of wellness programs in response to a request by a client, in accordance with an embodiment of the present disclosure; and

FIG. 15 illustrates a flow chart of a workflow for considering one or more attributes of a wellness program, in accordance with an embodiment of the present disclosure.

It should be understood that the appended drawings are not necessarily to scale, presenting a somewhat simplified representation of various features illustrative of the basic principles of the invention. The specific design features of the present invention as disclosed herein, including, for example, specific dimensions, orientations, locations, and shapes will be determined in part by the particular intended application and use environment.

In the figures, reference numbers refer to the same or equivalent parts of the present invention throughout the several figures of the drawing.

### DETAILED DESCRIPTION

One aspect of the present disclosure provides systems and methods for matching a client with a coach. A client is a person who wants to use the coach to improve an aspect of their life. For instance, in some embodiments, the client is a trainee that wants to improve performance of a particular activity, such as mediation. In some embodiments, the client is an apprentice coach that wants to improve performance for conducting a respective wellness program, thereby improving the life of the client. On the other hand, the coach is a qualified specialist who offers one or more wellness programs (e.g., services) for clients. The disclosed methods are performed using a computer system, such as a wellness computer system and/or a remote client device. In some embodiments, a request for a wellness program is received. A non-limiting example of the request for the wellness program includes a plurality of responses to a survey, in which the plurality of responses is assigned to a client through an electronic request to the wellness program. Another non-limiting example of the request for the wellness program includes evaluating a transcript of a conversation between the client and a respective coach in order to assign a set of attributes to the client. In some embodiments, the plurality of responses includes at least two answers provided by the client to at least two questions. In some embodiments, the at least two questions includes one or more questions related to health and/or medical concerns of the client (e.g., medical history of the client, undiagnosed concerns of the client, etc.), one or more questions related to certain goals of the client (e.g., a first goal satisfied when the wellness program is deemed complete, a second goal satisfied when the client satisfies a threshold condition such as a threshold weight loss, etc.), one or more questions related to certain focuses of the client (e.g., lifestyle focus, sport focus, wellness focus, etc.), one or more questions related to personal characteristics of the client (e.g., for a corresponding user profile), or a combination thereof. Accordingly, the at least two answers provided by the client collectively form a set of attributes that is assigned to the client. In some embodiments, at least the set of attributes is processed by a plurality of computational models to produce a result, such as a recommendation of a revised set of attributes. For instance, in some embodiments, the plurality of computational models processes the set of attributes assigned to the respective client, one or more historical data sets (e.g., a first corresponding historical performance of a corresponding coach, a second historical performance of a respective wellness program, a third historical performance of a respective client, etc.), one or more corpus of communications (e.g., a first corpus of communications associated with certain scientific publications), or a combination thereof. In some embodiments, a plurality of wellness programs is obtained from this set of attributes and the plurality of data sets. In some embodiments, a plurality of coaches is obtained, for instance, based on the identity of the plurality of wellness programs.

In this way, the plurality of coaching profiles, the plurality of wellness programs, the set of attributes assigned to the first client, or the combination thereof is processed using the plurality of computational models to produce a respective result for each computational model in the plurality of computational models. In some embodiments, the respective result includes a quality index associated with a respective wellness program and/or a respective coach (e.g., an average achievement score weighted by client engagement) and/or a confidence value associated with the respective wellness program and/or the respective coach (e.g., a logarithmic function of a number of sessions of the respective wellness program). However, the present disclosure is not limited thereto. By collectively considering each respective result, a set of at least one coaching profile and at least one wellness program is produced. In some embodiments, this set of the at least one coaching profile and the at least one wellness program includes a list of the at least one coaching profile and the at least one wellness that are matched to the client (e.g., depending on the quality index and confidence values determined for each respective coaching profile). Accordingly, the at least one coaching profile and the at least one wellness in the set are a best match for the client, which ensure the client is provided with high quality coaching and further ensures the coach is provided with clients.

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, and components have not been described in detail so as not to unnecessarily obscure aspects of the embodiments.

It will also be understood that, although the terms first, second, etc. may be used herein to describe various elements, these elements should not be limited by these terms. These terms are only used to distinguish one element from another. For instance, a first digital chart could be termed a second digital chart, and, similarly, a second digital chart could be termed a first digital chart, without departing from the scope of the present disclosure. The first digital chart and the second digital chart are both digital charts, but they are not the same digital chart.

The terminology used in the present disclosure is for the purpose of describing particular embodiments only and is not intended to be limiting of the invention. As used in the description of the invention and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The foregoing description included example systems, methods, techniques, instruction sequences, and computing machine program products that embody illustrative implementations. For purposes of explanation, numerous specific details are set forth in order to provide an understanding of various implementations of the inventive subject matter. It will be evident, however, to those skilled in the art that implementations of the inventive subject matter may be practiced without these specific details. In general, well-known instruction instances, protocols, structures, and techniques have not been shown in detail.

The foregoing description, for purpose of explanation, has been described with reference to specific implementations. However, the illustrative discussions below are not intended to be exhaustive or to limit the implementations to the precise forms disclosed. Many modifications and variations are possible in view of the above teachings. The implementations are chosen and described in order to best explain the principles and their practical applications, to thereby enable others skilled in the art to best utilize the implementations and various implementations with various modifications as are suited to the particular use contemplated.

In the interest of clarity, not all of the routine features of the implementations described herein are shown and described. It will be appreciated that, in the development of any such actual implementation, numerous implementation-specific decisions are made in order to achieve the designer's specific goals, such as compliance with use case- and business-related constraints, and that these specific goals will vary from one implementation to another and from one designer to another. Moreover, it will be appreciated that such a design effort might be complex and time-consuming, but nevertheless be a routine undertaking of engineering for those of ordering skill in the art having the benefit of the present disclosure.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

As used herein, the term “about” or “approximately” can mean within an acceptable error range for the particular value as determined by one of ordinary skill in the art, which can depend in part on how the value is measured or determined, e.g., the limitations of the measurement system. For example, “about” can mean within 1 or more than 1 standard deviation, per the practice in the art. “About” can mean a range of ±20%, ±10%, ±5%, or ±1% of a given value. Where particular values are described in the application and claims, unless otherwise stated, the term “about” means within an acceptable error range for the particular value. The term “about” can have the meaning as commonly understood by one of ordinary skill in the art. The term “about” can refer to ±10%. The term “about” can refer to ±5%.

As used herein, the term “dynamically” means an ability to update a program while the program is currently running.

Additionally, the terms “client,” “patient,” “subject,” “end-user,” and “user” are used interchangeably herein unless expressly stated otherwise.

Furthermore, the terms “discipline” and “focus” are used interchangeably herein unless expressly stated otherwise.

Moreover, as used herein, the term “parameter” refers to any coefficient or, similarly, any value of an internal or external element (e.g., a weight and/or a hyperparameter) in an algorithm, model, regressor, and/or classifier that can affect (e.g., modify, tailor, and/or adjust) one or more inputs, outputs, and/or functions in the algorithm, model, regressor and/or classifier. For example, in some embodiments, a parameter refers to any coefficient, weight, and/or hyperparameter that can be used to control, modify, tailor, and/or adjust the behavior, learning, and/or performance of an algorithm, model, regressor, and/or classifier. In some instances, a parameter is used to increase or decrease the influence of an input (e.g., a feature) to an algorithm, model, regressor, and/or classifier. As a nonlimiting example, in some embodiments, a parameter is used to increase or decrease the influence of a node (e.g., of a neural network), where the node includes one or more activation functions. Assignment of parameters to specific inputs, outputs, and/or functions is not limited to any one paradigm for a given algorithm, model, regressor, and/or classifier but can be used in any suitable algorithm, model, regressor, and/or classifier architecture for a desired performance. In some embodiments, a parameter has a fixed value. In some embodiments, a value of a parameter is manually and/or automatically adjustable. In some embodiments, a value of a parameter is modified by a validation and/or training process for an algorithm, model, regressor, and/or classifier (e.g., by error minimization and/or backpropagation methods). In some embodiments, an algorithm, model, regressor, and/or classifier of the present disclosure includes a plurality of parameters. In some embodiments, the plurality of parameters is n parameters, where: n≥2; n≥5; n≥10; n≥25; n≥40; n≥50; n≥75; n≥100; n≥125; n≥150; n≥200; n≥225; n≥250; n≥350; n≥500; n≥600; n≥750; n≥1,000; n≥2,000; n≥4,000; n≥5,000; n≥7,500; n≥10,000; n≥20,000; n≥40,000; n≥75,000; n≥100,000; n≥200,000; n≥500,000, n≥1×10⁶, n≥5×10⁶, or n≥1×10⁷. In some embodiments n is between 10,000 and 1×10⁷, between 100,000 and 5×10⁶, or between 500,000 and 1×10⁶.

Furthermore, when a reference number is given an “i^{th}” denotation, the reference number refers to a generic component, set, or embodiment. For instance, a client device termed “client device i” refers to the i^{th} client device in a plurality of client devices (e.g., a client device 300-i in a plurality of client devices 300).

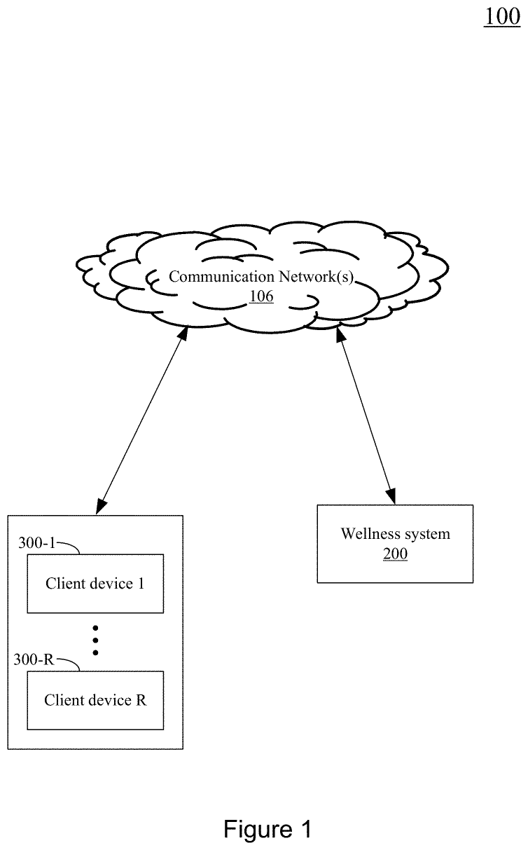

In the present disclosure, unless expressly stated otherwise, descriptions of devices and systems will include implementations of one or more computers. For instance, and for purposes of illustration in FIG. 1, a client device 300 is represented as single device that includes all the functionality of the client device 300. However, the present disclosure is not limited thereto. For instance, the functionality of the client device 300 may be spread across any number of networked computers and/or reside on each of several networked computers and/or by hosted on one or more virtual machines and/or containers at a remote location accessible across a communications network (e.g., communications network 106). One of skill in the art will appreciate that a wide array of different computer topologies is possible for the client device 300, and other devices and systems of the preset disclosure, and that all such topologies are within the scope of the present disclosure.

FIG. 1 depicts a block diagram of a distributed client-server system (e.g., distributed client-server system 100) according to some embodiments of the present disclosure. The system 100 at least facilitates hosting a plurality of wellness programs. Each wellness program is configured to be conducted by a client with the assistance of a coach. From this, the system 100 obtains one or more data sets associated with the client, the coach, a respective wellness program, or a combination thereof that is associated with a historical performance, such as when the coach and/or the client engages with the system 100 (e.g., during the respective wellness program).

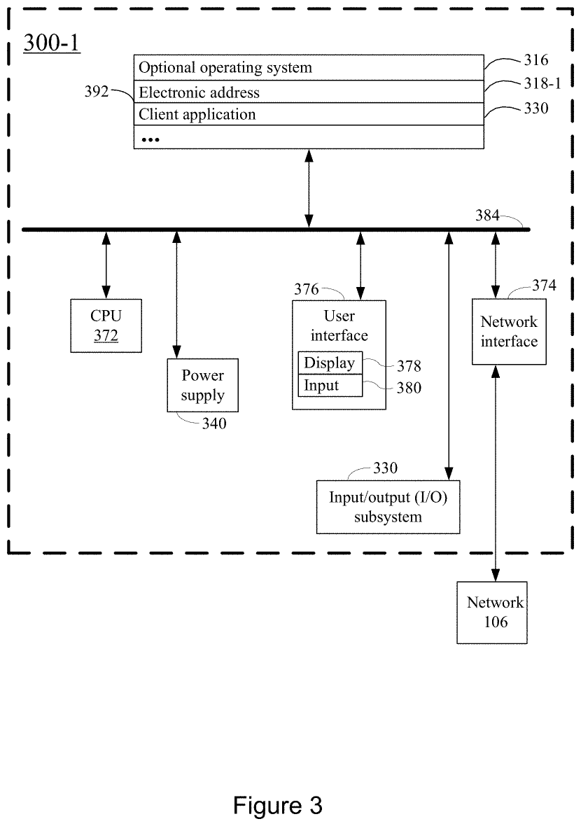

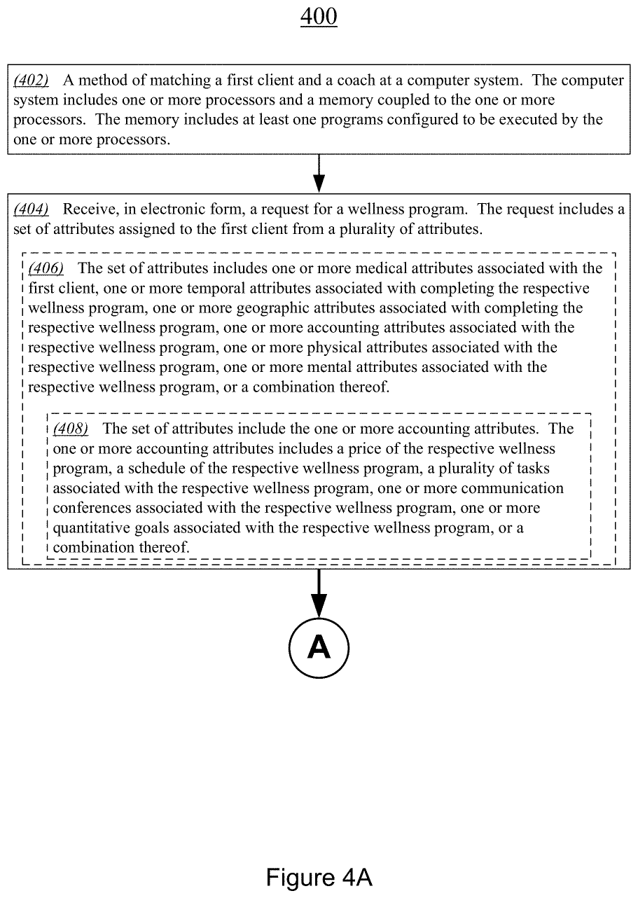

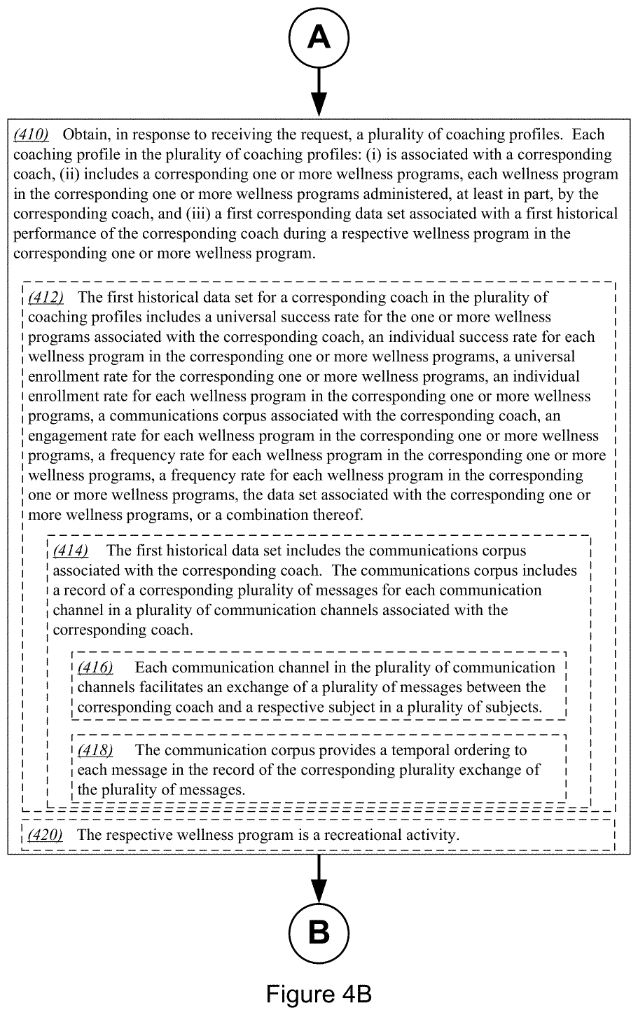

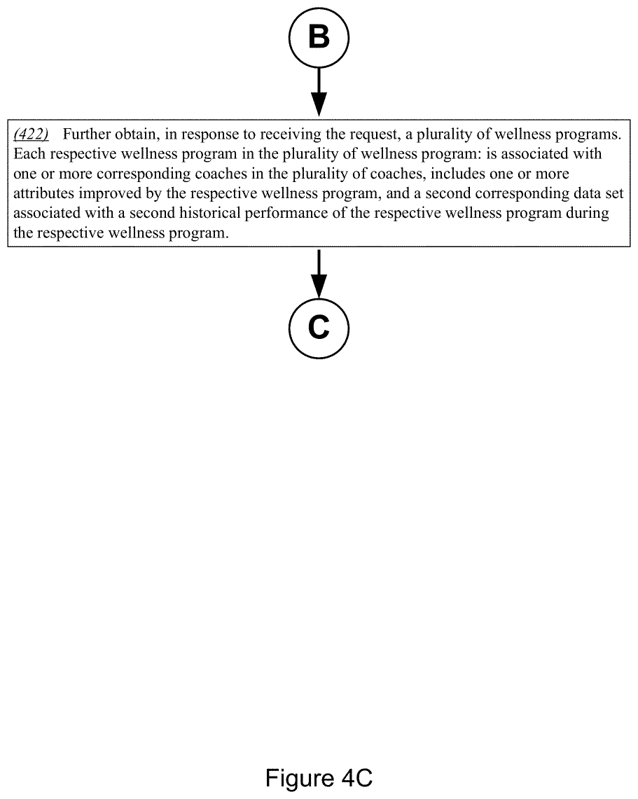

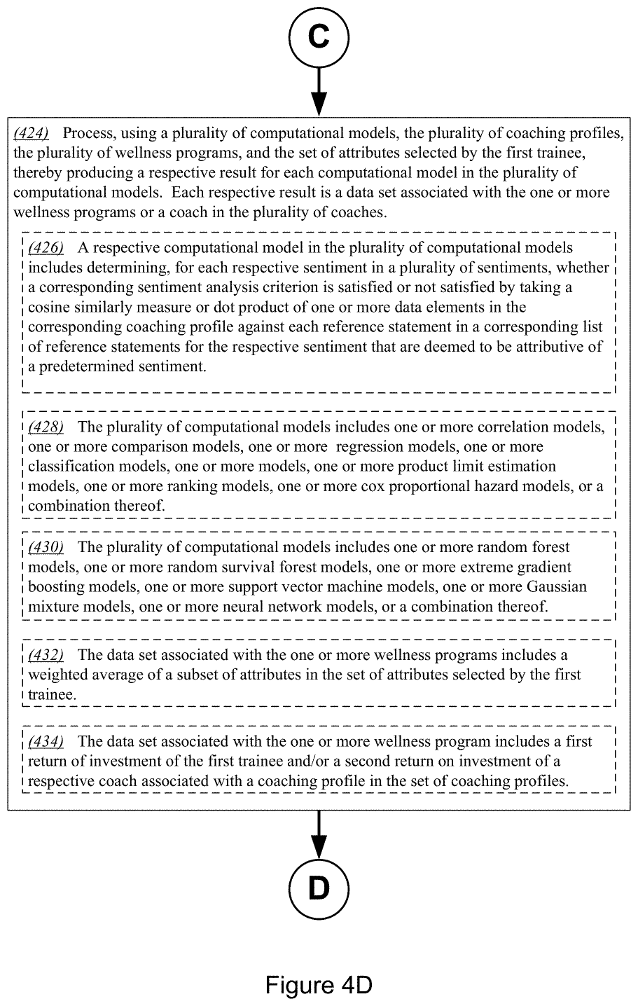

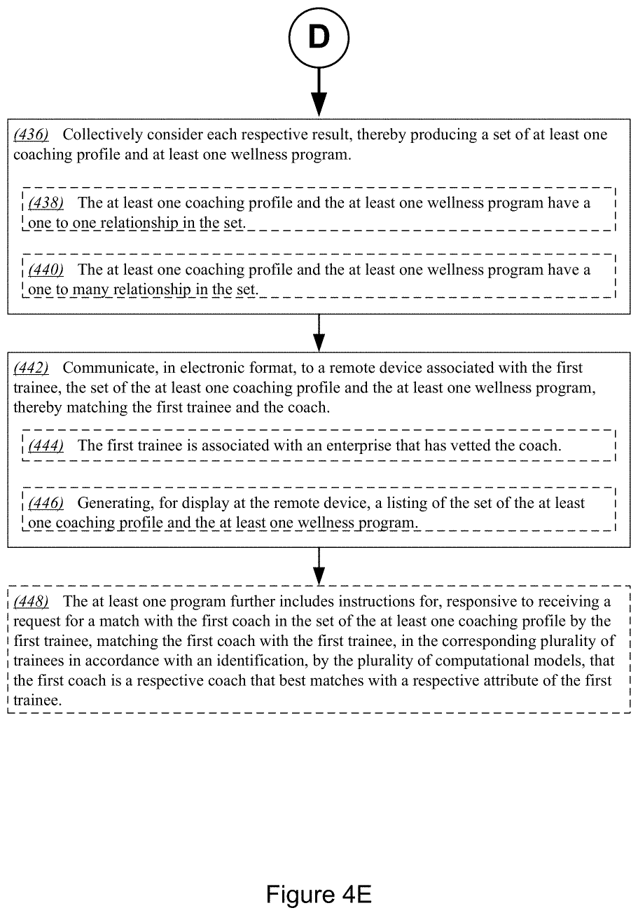

The system 100 facilitates hosting the plurality of wellness programs for a population of subjects (e.g., end-users associated with one or more client devices 300). The population of subjects includes a plurality of clients and a plurality of coaches. In some embodiments, the plurality of clients includes one or more apprentice coaches. In some embodiments, the plurality of clients includes one or more trainees. Furthermore, in some embodiments, the plurality of clients is associated with one or more entities, such as a corporation (e.g., block 444 of FIG. 4E). In some embodiments, the wellness program 210 is prepared at a first client device 300-1 by a coach, and then the wellness program 210 is provided, at least in part, to a client through a graphical user interface (GUI) (e.g., user interface 374 of FIG. 3) that is displayed through a display of a second client device 300-2 associated with the client (e.g., display 376 of FIG. 3). However, the present disclosure is not limited thereto. For instance, in some embodiments, the wellness program 210 is prepared at the first client device 300-1 by the coach, and then the wellness program 210 is provided between the coach and the client.

Of course, other topologies of the system 100 are possible. For instance, in some embodiments, any of the illustrated devices and systems can in fact constitute several computer systems that are linked together in a network or be a virtual machine and/or container in a cloud-computing environment. Moreover, rather than relying on a physical communications network 106, the illustrated devices and systems may wirelessly transmit information between each other. As such, the exemplary topology shown in FIG. 1 merely serves to describe the features of an embodiment of the present disclosure in a manner that will be readily understood to one of skill in the art.

In some embodiments, the communication network 106 optionally includes the Internet, one or more local area networks (LANs), one or more wide area networks (WANs), other types of networks, or a combination of such networks.

Examples of communication networks 106 include the World Wide Web (WWW), an intranet and/or a wireless network, such as a cellular telephone network, a wireless local area network (LAN) and/or a metropolitan area network (MAN), and other devices by wireless communication. The wireless communication optionally uses any of a plurality of communications standards, protocols and technologies, including Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (Wi-Fi) (e.g., IEEE 802.11a, IEEE 802.11ac, IEEE 802.11ax, IEEE 802.11b, IEEE 802.11g and/or IEEE 802.11n), voice over Internet Protocol (VoIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol, including communication protocols not yet developed as of the filing date of this document.

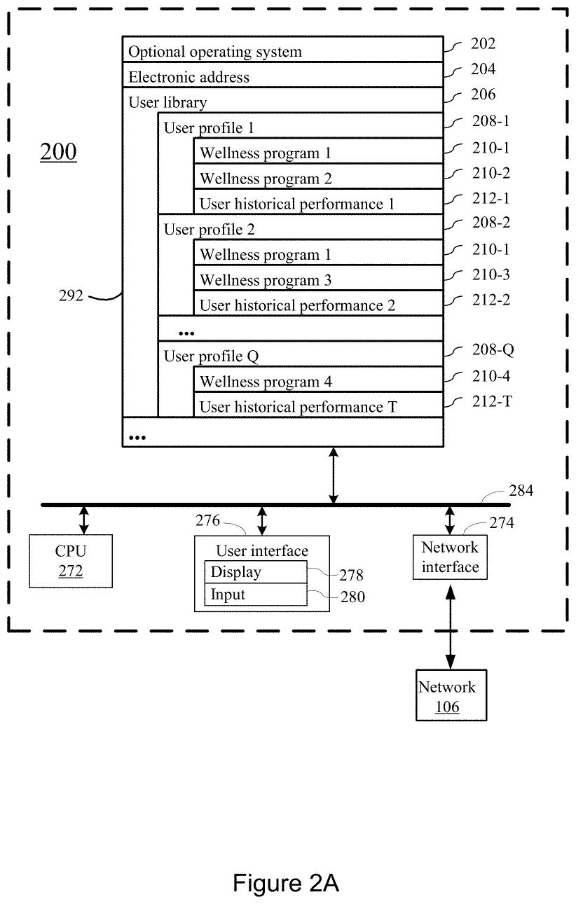

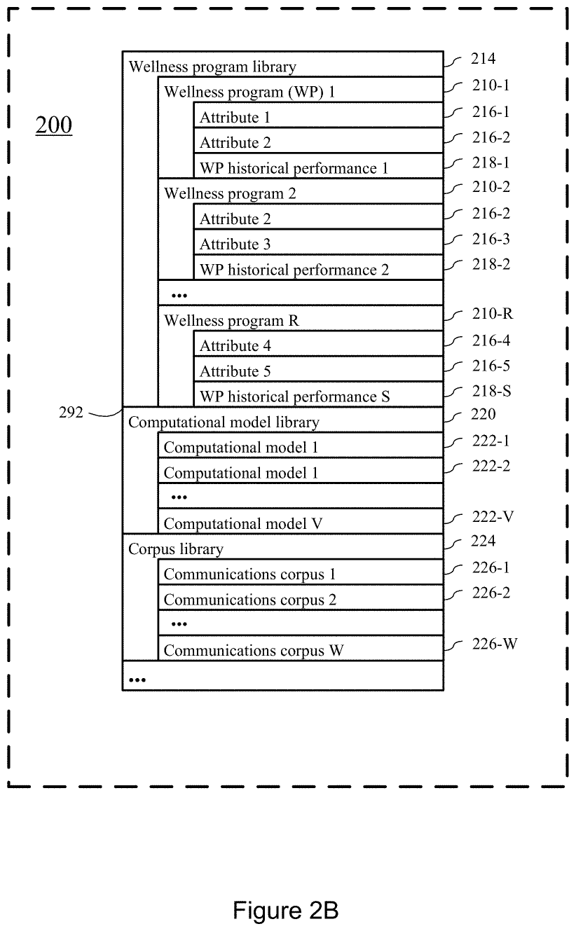

Now that a distributed client-server system 100 has generally been described, an exemplary wellness system 200 for hosting a plurality of wellness programs 210 will be described with reference to FIGS. 2A and 2B.

In various embodiments, the wellness system 200 includes one or more processing units (CPUs) 272, a network or other communications interface 274, and memory 292.

Memory 292 includes high-speed random access memory, such as DRAM, SRAM, DDR RAM, or other random access solid state memory devices, and optionally also includes non-volatile memory, such as one or more magnetic disk storage devices, optical disk storage devices, flash memory devices, or other non-volatile solid state storage devices. Memory 292 may optionally include one or more storage devices remotely located from the CPU(s) 272. Memory 292, or alternatively the non-volatile memory device(s) within memory 292, includes a non-transitory computer readable storage medium. Access to memory 292 by other components of the wellness system 200, such as the CPU(s) 272, is, optionally, controlled by a controller. In some embodiments, memory 292 can include mass storage that is remotely located with respect to the CPU(s) 272. In other words, some data stored in memory 292 may in fact be hosted on devices that are external to the wellness system 200, but that can be electronically accessed by the wellness system 200 over an Internet, intranet, or other form of network 106 or electronic cable using communication interface 2877.

In some embodiments, the memory 292 of the wellness system 200 for hosting the plurality of wellness programs 210 stores:
an operating system 202 (e.g., ANDROID, iOS, DARWIN, RTXC, LINUX, UNIX, OS X, WINDOWS, or an embedded operating system such as VxWorks) that includes procedures for handling various basic system services;
an electronic address 204 associated with the wellness system 200 that identifies the wellness system 200;
a user library 206 that stores a plurality of user profiles 208, each user profile is associated a corresponding user (e.g., a corresponding coach, a corresponding client, etc.) of the wellness program and further associated with one or more wellness programs 210 in the plurality of wellness programs 210 hosted by the wellness system 200 and with a user historical performance 212, such as when engaging with the wellness system 200;
a wellness program library 214 that stores the plurality of wellness programs 210 hosted by the wellness system 200, each wellness program 210 in the plurality of wellness programs 210 associated with a set of attributes 216 that is configured to define a focus and/or discipline of a respective wellness program 210;
a computational model library 220 that stores a plurality of computational models 222 for processing information and producing a data set as a result of this processing; and
a corpus library 224 that stores a plurality of communications corpora 226, each respective communications corpus 226 in the plurality of communications corpora 226 associated with a particular subject matter.

As indicated above, an electronic address 204 is associated with the wellness system 200. The electronic address 204 is utilized to at least uniquely identify the wellness system 200 from other devices and components of the distributed system 100 (e.g., uniquely identify wellness system 200 from second client device 300-2 and third client device 300-3). For instance, in some embodiments, the electronic address 204 is utilized to receive a request from a client device 300 to participate in a respective wellness program or the like.

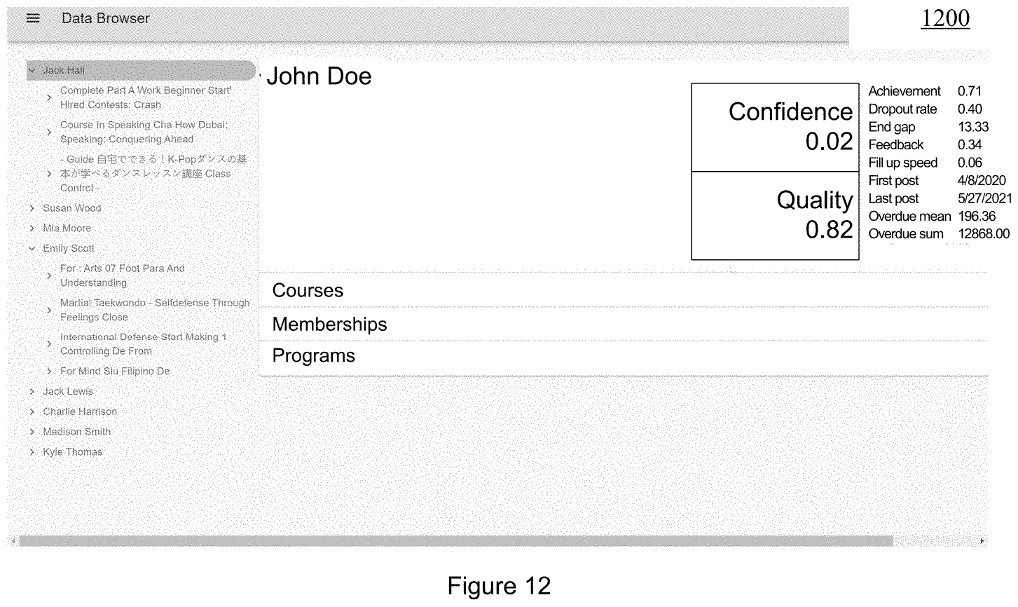

A user library 204 retains a plurality of user profiles 208. Each respective user profile 208 is associated with a corresponding user of the wellness system 200. In some such embodiments, each respective user profile includes an identifier that is configured to designate a type of user within the system 100, such as a respective coach that administrates and/or authors (e.g., creates) one or more wellness programs 210 hosted by the wellness system, one or more clients (e.g., a trainee or an apprentice coach) that will participate, or are presently participating in, a respective wellness program 210, or a combination thereof. Referring briefly to FIG. 12, a non-limiting example of a respective user profile 208 for a coach is provided, which includes a listing of one or more wellness programs associated with the coach (e.g., “Programs”) and one or more clients that are associated with the one or more wellness programs 210 (e.g., “Memberships”). For instance, in some embodiments, a respective user first customizes his or her profile (e.g., first user profile 208-1) at a client device 300 by providing a plurality of user login information, such as a password, an address (e.g., E-mail address, physical address, etc.), a personal name (e.g., a given name, a username, etc.), and the like. In some embodiments, the user profile 208 uniquely identifies the respective user within the wellness system 200, such that no two users are associated with a respective user profile 208. In this way, each user profile 208 allows the wellness system 200 to retain login information, privacy information, geographical data associated with the respective user (e.g., preferred location(s) for conducting a respective wellness program), biographical data associated with the respective user, or a combination thereof. In some such embodiments, the user profile 208 further identifies the corresponding user associated with the user profile 208 as a respective client or a coach, which allows for access to certain features of a client application 320 associated with the wellness system 200, such as an ability to create and/or edit a respective wellness program 210. However, the present disclosure is not limited thereto.

Moreover, in some embodiments, the user profile 208 includes a plurality of attributes 216 assigned to the corresponding user. For instance, in some embodiments, the user profile 208 includes at least 2 attributes 216 assigned to the corresponding user, at least 3 attributes assigned to the corresponding user, at least 5 attributes assigned to the corresponding user, at least 10 attributes assigned to the corresponding user, at least 15 attributes assigned to the corresponding user, at least 50 attributes assigned to the corresponding user, at least 100 attributes assigned to the corresponding user, at least 500 attributes assigned to the corresponding user, or a combination thereof.

In some embodiments, a first subset of attributes 216 in the plurality of attributes 216 is assigned to the corresponding user by the corresponding user, such as a name of the corresponding user, an age of the corresponding user, a gender of the corresponding user, a height of the corresponding user, a weight of the corresponding user (e.g., an initial weight of the user, a recently measured weight of the corresponding user, a goal weight of the corresponding user, etc.), and the like. In some embodiments, the first subset of attributes 216 includes at least 2 attributes, at least 5 attributes, at least 10 attributes, at least 20 attributes, at least 50 attributes, at least 100 attributes, or a combination thereof. In some embodiments, a second subset of attributes 216 in the plurality of attribute 216 is assigned to the corresponding user by the wellness system 200, such that the corresponding user is associated with the second set of attributes 216 but is restricted from modifying the second set of attributes. Non-limiting examples of a respective attribute in the second subset of attributes 216 include a feedback score of the corresponding user, a professional licenses status of the corresponding user, and the like. However, the present disclosure is not limited thereto. For instance, referring briefly to FIG. 12, in some embodiments, the second subset of attributes includes an achievement score of the corresponding user, a dropout rate of the corresponding user, a feedback score of the corresponding user, an engagement characteristic of the corresponding user (e.g., a first date of an initial post in a communication channel by the corresponding user, a second date of a most recent post in the communication channel by the corresponding user, etc.). In some embodiments, the second subset of attributes 216 includes at least 2 attributes, at least 5 attributes, at least 10 attributes, at least 20 attributes, at least 50 attributes, at least 100 attributes, or a combination thereof. In some embodiments, the plurality of attributes 216 includes one or more medical condition attributes associated with the corresponding user, such as one or more medical conditions exhibited by the user (e.g., smoking status, amputations, anxiety disorders, behavioral and/or emotional disorders, bipolar affective disorder, depression, dissociation and/or dissociative disorders, drugs and/or alcohol abuse disorder, eating disorders, obsessive compulsive disorder, paranoia, panic attacks, post-traumatic stress disorder, psychosis, social withdrawal, etc.). In some embodiments, the one or more medical condition attributes is assigned to the corresponding user by the corresponding user. In some embodiments, the one or more medical condition attributes is assigned to the corresponding user based on an evaluation of a medical record of the corresponding user. Additionally, in some embodiments, a respective attribute in the plurality of attributes 216 associated with the user profile is assigned to the corresponding user by a subject other than the corresponding user (e.g., by an administrator of the wellness system, a clinician associated with the corresponding user, etc.).

Furthermore, in some embodiments, the plurality of attributes 216 includes a profession of the corresponding user (e.g., professional volleyball player, professional volleyball coach, shaman, music teacher, etc.), a lifestyle (e.g., nutritional lifestyle, exercise lifestyle, sleep lifestyle, stress management lifestyle, social contact lifestyle, etc.), and the like.

Furthermore, some embodiments, the user profile 208 allows the coach to designate one or more specialties of the corresponding coach (e.g., stress management specialty, situation diffusion specialty, etc.), an educational background of the corresponding user, one or more accolades associated with the corresponding user (e.g., awards), or a combination thereof. In some such embodiments, this designated information provided by the corresponding information provides qualifying information, such as either to qualify as a respective coach within the wellness system 200 or for presentation (e.g., advertisement) to clients within the wellness system 200.

In some embodiments, the plurality of user profiles 208 includes at least 5 coach profiles, at least 10 coach profiles, at least 25 coach profiles, at least 50 coach profiles, at least 100 coach profiles, at least 150 coach profiles, at least 250 coach profiles, at least 600 coach profiles, at least 1,000 coach profiles, at least 2,500 coach profiles, at least 5,000 coach profiles, at least 10,000 coach profiles, at least 100,000 coach profiles, or a combination thereof.

In some embodiments, the plurality of user profiles 208 includes at least 5 client profiles, at least 10 client profiles, at least 25 client profiles, at least 50 client profiles, at least 100 client profiles, at least 150 client profiles, at least 250 client profiles, at least 600 client profiles, at least 1,000 client profiles, at least 2,500 client profiles, at least 5,000 client profiles, at least 10,000 client profiles, at least 100,000 client profiles, at least 1,000,000 client profiles, or a combination thereof. In some embodiments, a number of client profiles in the plurality of user profiles 208 is greater than a number of coaching profiles in the plurality of user profiles 208. In some embodiments, the number of client profiles in the plurality of user profiles 208 is ten times, twenty time, fifty time, sixty times, a hundred times, or a thousand times greater than the number of coaching profiles in the plurality of user profiles 208.

In some embodiments, the user profile 208 includes some or all of a corresponding user historical performance 212 associated with the user profile 208. Each corresponding user historical performance 212 is a data set that is at least associated with a performance of one or more wellness programs 210 by the respective user, such as how much of a respective wellness program 210 the respective user has completed. In some embodiments, the performance of the one or more wellness programs 210 by the user is provided by one or more computational models in a plurality of computational models (e.g., 2 computational models, five computational models, etc.). In some embodiments, this performance of the one or more wellness programs 210 includes communications sent to the respective user and/or provided by the respective user, which allows for the wellness system 200 to evaluate how the respective user communicates with other users within the respective wellness program 210. However, the present disclosure is not limited thereto. In some embodiments, by storing the corresponding user historical performance 212, a respective coach is constantly challenged to engage with the wellness system 200 and, therefore, improve his or her skills and/or the corresponding one or more wellness programs 210 associated with the respective coach since subjective and/or objective historical data 212 from past performances by the respective coach and/or the client associated with the respective coach is stored by the wellness system 200 and used to match a future client with the respective coach. In this way, coaches that have high rated past performances and clients that complete and improve from the one or more wellness programs 210 of the respective coach improve the ability of the respective coach to match with the future client.

In some embodiments, the corresponding user historical performance 212 includes a plurality of task associated with one or more wellness programs 210 further associated with the corresponding user of the corresponding user profile 208. Each task is a unit of what the corresponding user must do to deem a portion including some or all of a respective wellness program 210 complete. Non-limiting examples of such tasks include completing a session, an event, a challenge, and the like. In some embodiments, each task is fixed to the respective wellness program 210 by the corresponding coach that is administering and/or authored the respective wellness program 210. In some embodiments, a respective task is associated with a corresponding attribute 216. For instance, consider a first task of completing a number of push-ups that is associated with a first attribute 216-1 for subjects with healthy upper bodies. However, the present disclosure is not limited thereto. In some embodiments, the plurality of tasks includes at least 5 tasks, at least 10 tasks, at least 20 tasks, at least 30 tasks, at least 60 tasks, at least 100 tasks, at least 250 tasks, or a combination thereof.

In some embodiments, the corresponding user historical performance 212 includes a plurality of achievements associated with the corresponding user. In some embodiments, the plurality of achievements represents what the user earned after the respective wellness program 210 is deemed complete by the wellness system. For instance, in some embodiments, each achieve is a data structure that supports a step function to reach a threshold value associated with a goal. In some embodiments, a respective achievement is associated with a corresponding attribute 216. For instance, consider a first achievement associated with a second weight loss attribute 216-2. This first achievement is configured by the corresponding user of wanting to lose 50 pounds and that the first achievement is to be completed in five equal steps. Here, the corresponding user historical performance 212 includes at least that the first user is 50% of this first achievement. However, the present disclosure is not limited thereto. In some embodiments, the plurality of achievements is processed by one or more computational models to determine a resultant data set that is an achievement score of the corresponding user, such as a first value (e.g., percentage) of total achievements deemed complete by the corresponding, a ratio of a first number of achievements deemed complete by the corresponding user against a second number of achievements attempted by the corresponding user, and the like.

In some embodiments, the corresponding user historical performance 212 for each respective user is collectively considered as a total user historical performance and then utilized by the plurality of computational models to determine one or more results of the systems and methods of the present disclosure. For instance, in some embodiments, the total user historical performance is a data set that includes a total client success (e.g., a sum across all clients of client success that is a sum of session success across all sessions). In some embodiments, the total user historical performance includes a session success of passing a respective wellness program 210 by a respective client. In some embodiments, the session success is based on time with at least one post in a communication channel, a number of tasks deemed complete (e.g., assuming uniform task distribution across the respective wellness program 210), a weighted average of goal achievements where one or more goal achievements are expressed (e.g., determined and/or presented) and weighted by the respective client before beginning the respective wellness program. However, the present disclosure is not limited thereto.

In some embodiments, the total user historical performance is a data set that includes a total coach success, a review score, and the like. In some embodiments, the review score is determined based on feedback by a plurality of clients that the wellness system 200 and/or the respective coach deems to complete the respective wellness program. In some embodiments, the review score provided for the respective coach by each client includes a result score (e.g., weighted at about 50%), a competence score (e.g., weighted at about 30%), an attention (e.g., weighted at about 10%), a manners and ease of communication (e.g., weighted at about 5%), a recommendation to other (e.g., weighted at about 5%), or a combination thereof.

In some embodiments, the total user historical performance includes a ranking as an additional source of data for the wellness system 200, such as to determine a popularity result of a respective wellness program and/or a respective coach using the plurality of computation models 222. In some embodiments, the plurality of coaches is ranked based on: a number of clients follow the corresponding coach; a number of sessions deemed completed for the corresponding coach, a number of wellness programs deemed completed and/or associated with the corresponding coach, a score of activity engaging with the wellness system 200 and/or a communication channel, or a combination thereof. In some embodiments, the wellness programs 210 are ranked by a number of sessions deemed completed for the corresponding wellness program 210, a length of the corresponding wellness program 210, a number of tasks deemed completed for the corresponding wellness program 210, a function of task and length of the corresponding wellness program, or a combination thereof (e.g., mean task length).

Referring to FIG. 2B, a wellness program library 214 is configured to store a plurality of wellness programs 210. Each wellness program 210 in the plurality of wellness programs is a program that is configured for an improvement of a respective client that engages with and completes the wellness program 210. In typical embodiments, a respective wellness program 210 is created at a client device 300 by user of the wellness system 200 that is a coach and then retained by the wellness system. Accordingly, in some embodiments, the respective wellness program 210 is a structured series of events, or tasks, that the respective client engages with to complete the respective wellness program 210. However, the present disclosure is not limited thereto. For instance, in some embodiments, the respective wellness program 210 is created by an administrator of the wellness system 200, such as a host of a client application 320.

In some embodiments, the plurality of wellness programs 210 includes at least 5 wellness programs, at least 10 wellness programs, at least 25 wellness programs, at least 50 wellness programs, at least 100 wellness programs, at least 150 wellness programs, at least 250 wellness programs, at least 600 wellness programs, at least 1,000 wellness programs, at least 2,500 wellness programs, at least 5,000 wellness programs, at least 10,000 wellness programs, at least 100,000 wellness programs, at least 1,000,000 wellness programs or a combination thereof.

Each wellness program 210 includes a plurality of attributes 216 (e.g., first wellness program 210-1 includes first attribute 216-1 and second attribute 216-1, second wellness program 210-2 includes second attribute 216-2 and third attribute 216-3, etc.). In some embodiments, each attribute 216 associated with a respective wellness program 210 defines a unique characteristic of the respective wellness program 210. In some embodiments, a respective attribute associated with the respective wellness program 210 is assigned to the respective wellness program by a client, by an administrator of the wellness system, by a coach, by a computational model, or a combination thereof. From the attributes 216, a user, such as a client user and/or an entity user, of the wellness system 200 is allowed to search for a respective coach and/or a respective wellness program by way of the attributes 216. For instance, in some embodiments, the unique characteristic is a discipline of the wellness program 210, such as a sports discipline (e.g., baseball, basketball, football, soccer, stretching, tennis, yoga, etc.), a health discipline (e.g., physical therapy, emotional therapy, etc.), and the like.

In some embodiments, the plurality of attributes 216 includes at least 5 attributes, at least 10 attributes, at least 25 attributes, at least 50 attributes, at least 100 attributes, at least 150 attributes, at least 250 attributes, at least 600 attributes, at least 1,000 attributes, at least 2,500 attributes, at least 5,000 attributes, at least 10,000 attributes, at least 100,000 attributes, at least 1,000,000 attributes, or a combination thereof.

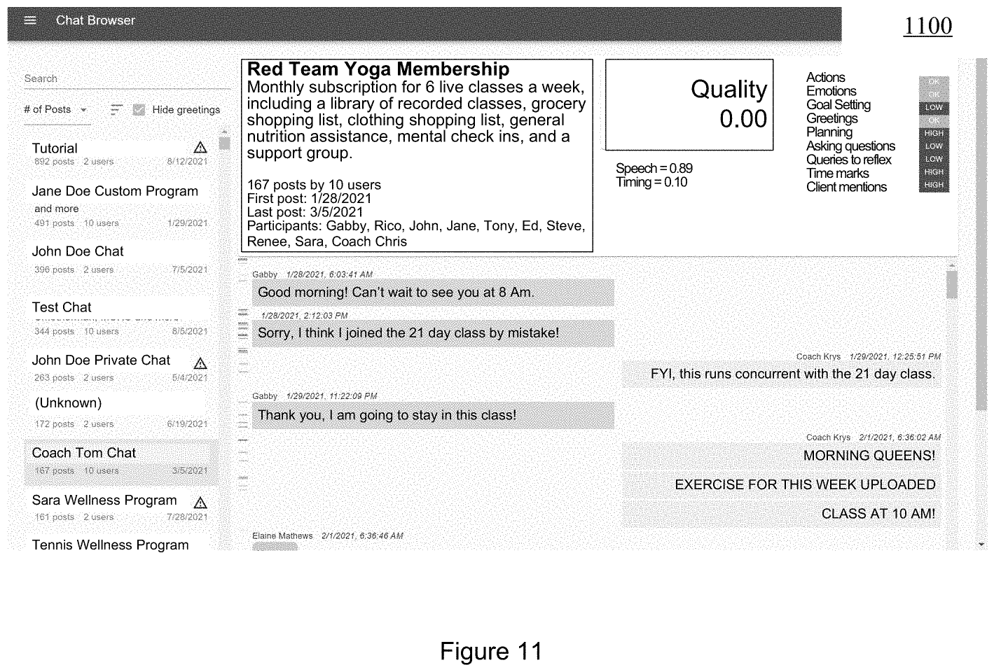

In some embodiments, when creating or editing a respective wellness program 210, the coach is able to specify a title of the respective wellness program 210 (e.g., “Red Team Yoga” of FIG. 11), a description of the respective wellness program 210 (e.g., “Monthly subscription for 6 live classes a week[ . . . ]” of FIG. 11), a focus of the respective wellness program 210, a length of the respective wellness program 210, a price of the respective wellness program 210, one or more data files of the respective wellness program 210, or a combination thereof. In some embodiments, the focus of the respective wellness program 210 is the attributes 216 of respective wellness program 210 is associated with and, optionally, one or more weights assigned to every attribute. In some embodiments, the length of the respective wellness program 210 is a period of time required to pass the respective wellness program, such as five months and/or ten sessions with the coach. In some embodiments, each coach is also provided with a set of goals to optimize for the respective wellness program 210. In some embodiments, the set of goals includes a number of completed sessions with a client, a total number of clients engaged with the respective wellness program 210 (e.g., instantaneously, collectively, etc.), profits earned from providing the respective wellness program, a popularity index of the respective wellness program, a quality index of the respective wellness program 210, or a combination thereof. In some embodiments, the coach is able to combine a respective goal in the set of goals by assigning, for example, a corresponding weight to each respective goal.

In some embodiments, a respective attribute 216 is configured to define when a wellness program 210 is deemed complete. As a non-limiting example, a first attribute 216-1 is configured to deem a first wellness program 210-1 complete for a respective client when the respective client has satisfied a threshold number of tasks, such as 80% of tasks. However, the present disclosure is not limited thereto.

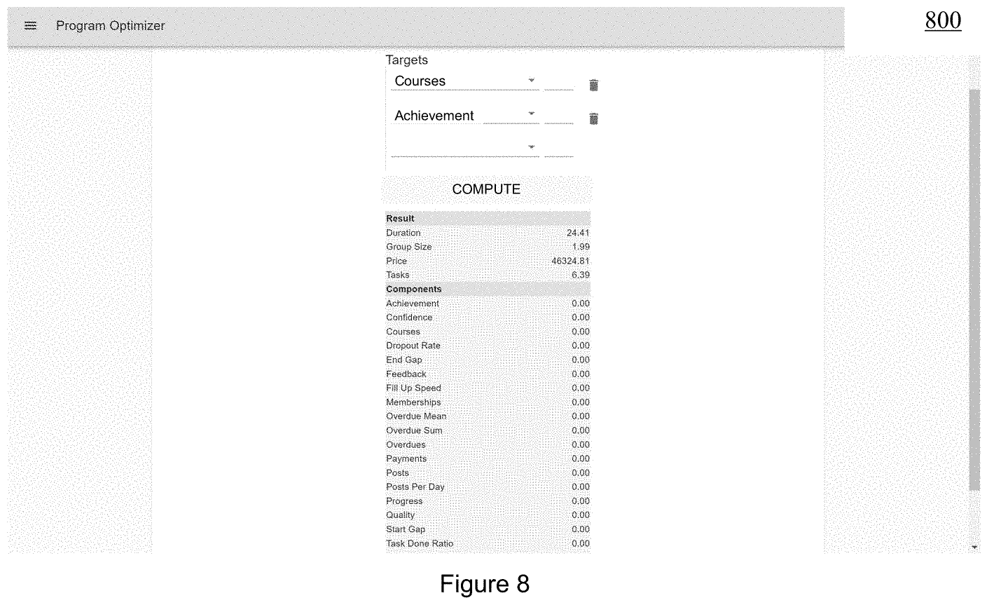

Each respective wellness program 210 includes a wellness program historical performance data set 216 that describes a performance of each respective client and/or each coach associated with the respective wellness program 210. For instance, in some embodiments, after completing the respective wellness program 210, the client is provided a survey for feedback on the respective wellness program 210 and/or a coach of the respective wellness program. In some embodiments, the feedback provided by the client includes one or more results in an activity of the respective wellness program 210 (e.g., scores from a tennis match), materials provided during the respective wellness program 210, a score of manners of the coach, a score of ease of communication with the coach, and the like. Referring briefly to FIG. 8, in some embodiments, the wellness program historical performance data set 216 include a temporal duration of the respective wellness program 210 (e.g., total time spent by the coach and/or one or more clients engaging with the respective wellness program 210, etc.), a confidence index of the respective wellness program 210, a client dropout rate of the respective wellness program 210, a rate of reaching capacity of the respective wellness program 210, a number of engagements of the respective wellness program 210 (e.g., number of posts per day within a communication channel associated with the respective wellness program 210), or a combination thereof.

In some embodiments, the wellness program historical performance data set 216 includes a popularity index. In some embodiments, the popularity index is provided by a plurality of computational models 222 as a function of, at least, a number of views of the wellness program through a respective client application 310, a number of purchases of the respective wellness program 210, and a number of followers of the respective wellness program 210. In some embodiments, the popularity is a number equal to or greater than zero.

In some embodiments, the wellness system 200 includes a computational model library 220 that stores a plurality of computational models 222 (e.g., classifiers, regressors, etc.). In some embodiments, the plurality of computational models 222 includes at least 5 computational models, at least 10 computational models, at least 25 computational models, at least 50 computational models, at least 100 computational models, at least 150 computational models, at least 250 computational models, at least 600 computational models, at least 1,000 computational models, at least 2,500 computational models, at least 5,000 computational models, at least 10,000 computational models, at least 100,000 computational models, or a combination thereof.

In some embodiments, the computational model 222 is implemented as an artificial intelligence engine. For instance, in some embodiments, the computational model includes one or more gradient boosting models, one or more random forest models, one or more neural networks (NN), one or more regression models, one or more Naïve Bayes models, one or more machine learning algorithms (MLA), or a combination thereof. In some embodiments, a MLA or a NN is trained from a training data set (e.g., a first training data set including a respective user historical performance 212 and/or a wellness program historical performance 218 or a combination thereof) that includes one or more features identified from a data set. By way of example, in some embodiments, the training data set includes data associated with a first user profile 208-1 and data associated with user tendencies when engaging a first wellness program 210-1. MLAs include supervised algorithms (such as algorithms where the features/classifications in the data set are annotated) using linear regression, logistic regression, decision trees, classification and regression trees, Naïve Bayes, nearest neighbor clustering; unsupervised algorithms (such as algorithms where no features/classification in the data set are annotated) using, for instance, means clustering, principal component analysis, random forest, adaptive boosting; and semi-supervised algorithms (such as algorithms where an incomplete number of features/classifications in the data set are annotated) using generative approach (such as a mixture of Gaussian distributions, mixture of multinomial distributions, hidden Markov models), low density separation, graph-based approaches (such as minimum cut, harmonic function, manifold regularization, etc.), heuristic approaches, or support vector machines. In some embodiments, the supervision of a respective computational model is performed by a medical practitioner associated with a user of a client device 300 that utilizes the systems and methods of the present disclosure.

In some embodiments, a probabilistic model is used in the methods and systems described herein, e.g., as a component model of an ensemble computational model 222. Probabilistic models employ random variables and probability distributions to a model for a phenomenon, e.g., the presence of a feature state, fraction, etc. Probabilistic models provide a probability distribution as a solution. Generally, probabilistic models can be classified as either graphical models (such as Bayesian networks, causal inference models, and Markov networks) or Stochastic models.

Probabilistic graphical models (PGMs) are probabilistic models for which a graph expresses a conditional dependence structure between random variables, encoding a distribution over a multi-dimensional space. One type of PGM is a Bayesian network, which is probabilistic graphical model that represents a set of variables and their conditional dependencies via a directed acyclic graph (DAG), according to Bayesian analysis. Briefly, given data x and parameter θ, Bayesian analysis uses a prior probability (a prior) p(θ) and a likelihood p(x|θ) to compute a posterior probability p(θ|x)∝p(x|θ) p(θ). Methods for learning Bayesian Networks are described, for example, in Castillo E, et al., “Learning Bayesian Networks,” Expert Systems and Probabilistic Network Models, Monographs in computer science, New York: Springer-Verlag, pp. 481-528, ISBN 978-0-387-94858-4, which is incorporated herein by reference, in its entirety, for all purposes. Another type of PGM is a Markov network, which is a set of random variables having a Markov property described by an undirected graph. Markov properties include pairwise Markov properties, in which any two non-adjacent variables are conditionally independent given all other variables, local Markov properties, in which a variable is conditionally independent of all other variables given its neighbors, and global Markov properties, in which any two subsets of variables are conditionally independent given a separating subset.

Stochastic probabilistic models model pseudo-randomly changing systems, assuming that future states depend only on a current state, not the events that occurred before the current state, otherwise known as the Markov property. Stochastic probabilistic models include Markov chains and Hidden Markov models (HMM). Markov chains are models describing a sequence of possible events in which the probability of each event depends only on the state attained in the previous event. For information on learning and application of Markov chains see, for example, Gagniuc, Paul A. (2017). Markov Chains: From Theory to Implementation and Experimentation. USA, NJ: John Wiley & Sons. pp. 1-235. ISBN 978-1-119-38755-8, which is incorporated herein by reference, in its entirety, for all purposes. Hidden Markov models (HMM) assume that a property Xis dependent upon an unobservable (“hidden”) state Y that can be learned based on observation of the property. For review of Hidden Markov models see, for example, Rabiner and Juang, “An introduction to hidden Markov models,” IEEE ASSP Magazine, 3(1):4-16 (1986), which is incorporated herein by reference, in its entirety, for all purposes.

In some embodiments, a deep learning model is used as a computational model 222 in the methods and systems described herein, e.g., as a component model of an ensemble classifier or circulating tumor fraction estimation model. Deep learning models use multiple layers to extract higher-level features from input data.

In some embodiments, the deep learning model of the computational model 222 is a neural network (e.g., a convolutional neural network and/or a residual neural network). Neural network algorithms, also known as artificial neural networks (ANNs), include convolutional and/or residual neural network algorithms (deep learning algorithms). Neural networks can be machine learning algorithms that may be trained to map an input data set to an output data set, where the neural network comprises an interconnected group of nodes organized into multiple layers of nodes. For example, the neural network architecture may include at least an input layer, one or more hidden layers, and an output layer. In some embodiments, the neural network includes any total number of layers, and any number of hidden layers, where the hidden layers function as trainable feature extractors that allow mapping of a set of input data to an output value or set of output values. As used herein, a deep learning algorithm (DNN) can be a neural network that includes a plurality of hidden layers, e.g., two or more hidden layers. In some embodiments, each layer of the neural network includes a number of nodes (or “neurons”). A node can receive input that comes either directly from the input data or the output of nodes in previous layers, and perform a specific operation, e.g., a summation operation. In some embodiments, a connection from an input to a node is associated with a parameter (e.g., a weight and/or weighting factor). In some embodiments, the node may sum up the products of all pairs of inputs, xi, and their associated parameters. In some embodiments, the weighted sum is offset with a bias, b. In some embodiments, the output of a node or neuron is gated using a threshold or activation function, f, which may be a linear or non-linear function. The activation function may be, for example, a rectified linear unit (ReLU) activation function, a Leaky ReLU activation function, or other function such as a saturating hyperbolic tangent, identity, binary step, logistic, arcTan, softsign, parametric rectified linear unit, exponential linear unit, softPlus, bent identity, softExponential, Sinusoid, Sine, Gaussian, or sigmoid function, or any combination thereof.

The weighting factors, bias values, and threshold values, or other computational parameters of the neural network, may be “taught” or “learned” in a training phase using one or more sets of training data, such as a communications corpus 232 associated with a particular candidate subject. For example, in some embodiments, the parameters is trained using the input data from a training data set (e.g., first communications corpus 232-1 of FIG. 2) and a gradient descent or backward propagation method so that the output value(s) that the ANN computes are consistent with the examples included in the training data set. In some embodiments, the parameters are obtained from a back propagation neural network training process.

Any of a variety of neural networks may be suitable for use in evaluating a request for a wellness program (e.g., block 404 of FIG. 4A), obtaining a plurality of coaching profiles (e.g., user profiles 208 associated with a respective coach) (e.g., block 410 of FIG. 4B), further obtaining a plurality of wellness programs (e.g., block 422 of FIG. 4C), processing the plurality of coaching profiles (e.g., block 424 of FIG. 4D), considering each respective result provided by a corresponding computational model (e.g., a first computational model 222-1 evaluates a respective result provided by a second computational model 222-2) (e.g., block 436 of FIG. 4E), communicating and/or determining a set of at least one coaching profile and at least one wellness program (e.g., block 442 of FIG. 4E), matching a first client with a coach (e.g., block 488 of FIG. 4E), or a combination thereof. Examples can include, but are not limited to, feedforward neural networks, radial basis function networks, recurrent neural networks, residual neural networks, convolutional neural networks, residual convolutional neural networks, and the like, or any combination thereof. In some embodiments, the machine learning makes use of a pre-trained and/or transfer-learned ANN or deep learning architecture. Convolutional and/or residual neural networks can be used to extract the set of attributes 216 arising from the evaluation of the wellness program requests (e.g., block 404 of FIG. 4A), obtaining the plurality of coaching profiles (e.g., user profiles 208 associated with the respective coach) (e.g., block 410 of FIG. 4B), further obtaining the plurality of wellness programs (e.g., block 422 of FIG. 4C), processing the plurality of coaching profiles (e.g., block 424 of FIG. 4D), considering each respective result provided by a corresponding computational model (e.g., a first computational model 222-1 evaluates the respective result provided by a second computational model 222-2) (e.g., block 436 of FIG. 4E), communicating and/or determining a set of at least one coaching profile and at least one wellness program (e.g., block 442 of FIG. 4E), matching a first client with a coach (e.g., block 488 of FIG. 4E), or the combination thereof.

For instance, a deep neural network model includes an input layer, a plurality of individually parameterized (e.g., weighted) convolutional layers, and an output scorer. The parameters (e.g., weights) of each of the convolutional layers as well as the input layer contribute to the plurality of parameters (e.g., weights) associated with the deep neural network model. In some embodiments, at least 100 parameters, at least 1,000 parameters, at least 2,000 parameters or at least 5,000 parameters are associated with the deep neural network model. As such, deep neural network models require a computer to be used because they cannot be mentally solved. In other words, given an input to the model, the model output needs to be determined using a computer rather than mentally in such embodiments. See, for example, Krizhevsky et al., 2012, “Imagenet classification with deep convolutional neural networks,” in Advances in Neural Information Processing Systems 2, Pereira, Burges, Bottou, Weinberger, eds., pp. 1097-1105, Curran Associates, Inc.; Zeiler, 2012 “ADADELTA: an adaptive learning rate method,” CoRR, vol. abs/1212.5701; and Rumelhart et al., 1988, “Neurocomputing: Foundations of research,” ch. Learning Representations by Back-propagating Errors, pp. 696-699, Cambridge, Mass., USA: MIT Press, each of which is hereby incorporated by reference.

Neural network algorithms, including convolutional neural network algorithms, suitable for use as models are disclosed in, for example, Vincent et al., 2010, “Stacked denoising autoencoders: Learning useful representations in a deep network with a local denoising criterion,” J Mach Learn Res 11, pp. 3371-3408; Larochelle et al., 2009, “Exploring strategies for training deep neural networks,” J Mach Learn Res 10, pp. 1-40; and Hassoun, 1995, Fundamentals of Artificial Neural Networks, Massachusetts Institute of Technology, each of which is hereby incorporated by reference. Additional example neural networks suitable for use as models are disclosed in Duda et al., 2001, Pattern Classification, Second Edition, John Wiley & Sons, Inc., New York; and Hastie et al., 2001, The Elements of Statistical Learning, Springer-Verlag, New York, each of which is hereby incorporated by reference in its entirety. Additional example neural networks suitable for use as models are also described in Draghici, 2003, Data Analysis Tools for DNA Microarrays, Chapman & Hall/CRC; and Mount, 2001, Bioinformatics: sequence and genome analysis, Cold Spring Harbor Laboratory Press, Cold Spring Harbor, N.Y., each of which is hereby incorporated by reference in its entirety.

In some embodiments, a mixture model, also referred to herein as an admixture model, is used as a computational model 222 in the methods and systems described herein, e.g., as a component model of a computational model 222. Mixture models are probabilistic model for representing the presence of subpopulations within an overall population, without requiring that an observed data set should identify the sub-population to which an individual observation belongs. Given a sampling of parameter data from a mixture of distributions, e.g., term occurrence, parts of speech, and financial model distributions of the parameters over each distribution separately, several techniques can be used to determine the parameters of the particular mixture of distributions. These techniques include maximum likelihood estimation (e.g., expectation maximization), application of Bayes' theorem on posterior sampling of the mixture of distributions (e.g., via a Markov chain Monte Carlo algorithm such as Gibbs sampling), moment matching, and several graphical methodologies. For a review of the use of mixture models see, for example, Titterington, D et al., “Statistical Analysis of Finite Mixture Distributions,” Wiley ISBN 978-0-471-90763-3 (1985), which is incorporated herein by reference, in its entirety, for all purposes.

Logistic regression algorithms suitable for use as computational models 222 are disclosed, for example, in Agresti, An Introduction to Categorical Data Analysis, 1996, Chapter 5, pp. 103-144, John Wiley & Son, New York, which is hereby incorporated by reference.

Neural network algorithms, including convolutional neural network algorithms, suitable for use as computational models 222 are disclosed in, for example, Vincent et al., 2010, “Stacked denoising autoencoders: Learning useful representations in a deep network with a local denoising criterion,” J Mach Learn Res 11, pp. 3371-3408; Larochelle et al., 2009, “Exploring strategies for training deep neural networks,” J Mach Learn Res 10, pp. 1-40; and Hassoun, 1995, Fundamentals of Artificial Neural Networks, Massachusetts Institute of Technology, each of which is hereby incorporated by reference. A neural network has a layered structure that includes a layer of input units (and the bias) connected by a layer of weights to a layer of output units. For regression, the layer of output units typically includes just one output unit. However, neural networks can handle multiple quantitative responses in a seamless fashion. In multilayer neural networks, there are input units (input layer), hidden units (hidden layer), and output units (output layer). There is, furthermore, a single bias unit that is connected to each unit other than the input units. Additional example neural networks suitable for use as computational models 222 are disclosed in Duda et al., 2001, Pattern Classification, Second Edition, John Wiley & Sons, Inc., New York; and Hastie et al., 2001, The Elements of Statistical Learning, Springer-Verlag, New York, each of which is hereby incorporated by reference in its entirety. Additional example neural networks suitable for use as classifiers are also described in Draghici, 2003, Data Analysis Tools for DNA Microarrays, Chapman & Hall/CRC; and Mount, 2001, Bioinformatics: sequence and genome analysis, Cold Spring Harbor Laboratory Press, Cold Spring Harbor, N.Y., each of which is hereby incorporated by reference in its entirety.

SVM algorithms suitable for use as computational models 222 are described in, for example, Cristianini and Shawe-Taylor, 2000, “An Introduction to Support Vector Machines,” Cambridge University Press, Cambridge; Boser et al., 1992, “A training algorithm for optimal margin classifiers,” in Proceedings of the 5^{th} Annual ACM Workshop on Computational Learning Theory, ACM Press, Pittsburgh, Pa., pp. 142-152; Vapnik, 1998, Statistical Learning Theory, Wiley, New York; Mount, 2001, Bioinformatics: sequence and genome analysis, Cold Spring Harbor Laboratory Press, Cold Spring Harbor, N.Y.; Duda, Pattern Classification, Second Edition, 2001, John Wiley & Sons, Inc., pp. 259, 262-265; and Hastie, 2001, The Elements of Statistical Learning, Springer, New York; and Furey et al., 2000, Bioinformatics 16, 906-914, each of which is hereby incorporated by reference in its entirety. When used for classification of textual data in a respective communication 240, SVMs separate a given set of binary labeled data training set (e.g., a first and second term condition of each respective term in a plurality of terms in a communications corpus 232) with a hyperplane that is maximally distant from the labeled data. For cases in which no linear separation is possible, SVMs can work in combination with the technique of kernels, which automatically realize a non-linear mapping to a feature space. The hyperplane found by the SVM in feature space corresponds to a non-linear decision boundary in the input space.

Naïve Bayes classifiers suitable for use as computational models 222 are disclosed, for example, in Ng et al., 2002, “On discriminative vs. generative classifiers: A comparison of logistic regression and naive Bayes,” Advances in Neural Information Processing Systems, 14, which is hereby incorporated by reference.

Decision trees algorithms suitable for use as computational models 222 are described in, for example, Duda, 2001, Pattern Classification, John Wiley & Sons, Inc., New York, pp. 395-396, which is hereby incorporated by reference. Tree-based methods partition the feature space into a set of rectangles, and then fit a model (like a constant) in each one. In some embodiments, the decision tree is random forest regression. One specific algorithm that can be used as a computational model 222 is a classification and regression tree (CART). Other examples of specific decision tree algorithms that can be used as computational models 222 include, but are not limited to, ID3, C4.5, MART, and Random Forests. CART, ID3, and C4.5 are described in Duda, 2001, Pattern Classification, John Wiley & Sons, Inc., New York. pp. 396-408 and pp. 411-412, which is hereby incorporated by reference. CART, MART, and C4.5 are described in Hastie et al., 2001, The Elements of Statistical Learning, Springer-Verlag, New York, Chapter 9, which is hereby incorporated by reference in its entirety. Random Forests are described in Breiman, 1999, “Random Forests—Random Features,” Technical Report 567, Statistics Department, U.C. Berkeley, September 1999, which is hereby incorporated by reference in its entirety.

Clustering algorithms suitable for use as computational models 222 are described, for example, at pages 211-256 of Duda and Hart, Pattern Classification and Scene Analysis, 1973, John Wiley & Sons, Inc., New York, (hereinafter “Duda 1973”) which is hereby incorporated by reference in its entirety. As set forth in Section 6.7 of Duda 1973, the clustering problem is described as one of finding natural groupings in a dataset. To identify natural groupings, two issues are addressed. First, a way to measure similarity (or dissimilarity) between two samples is determined. This metric (similarity measure) is used to ensure that the samples in one cluster are more like one another than they are to samples in other clusters. Second, a mechanism for partitioning the data into clusters using the similarity measure is determined. Similarity measures are discussed in Section 6.7 of Duda 1973, where it is stated that one way to begin a clustering investigation is to define a distance function and to compute the matrix of distances between all pairs of samples in a dataset. If distance is a good measure of similarity, then the distance between samples in the same cluster will be significantly less than the distance between samples in different clusters. However, as stated on page 215 of Duda 1973, clustering does not require the use of a distance metric. For example, a nonmetric similarity function s(x, x′) can be used to compare two vectors x and x′. Conventionally, s(x, x′) is a symmetric function whose value is large when x and x′ are somehow “similar.” An example of a nonmetric similarity function s(x, x′) is provided on page 216 of Duda 1973.

In some embodiments, the similarity includes a first similarity between two or more user profiles 208 (e.g., to determine a first recommendation for one or more wellness programs 208 deemed complete by similar clients). In some embodiments, the similarity includes a second similarity between at least two sets of attributes 216, such as two sets of goals (e.g., to determine a second recommendation for one or more wellness programs 210 with one or more attribute 216 similar to the at least to sets of attributes 216). In some embodiments, the similarity includes a third similarity between at least two sets of attributes 216 and at least two sets of wellness programs 216 (e.g., to determine a third recommendation for one or more wellness programs 216 that satisfy each attribute in a set of attributes 216 assigned to the client). In some embodiments, the similarity includes a fourth similarity between at least two texts in one or more corpus of communications 226 (e.g., to determine a fourth recommendation for one or more clusters of a plurality of wellness programs 210, a plurality of communication channels, a plurality of material, or a combination there). In some embodiments, the similarity includes a fifth similarity between at least two wellness programs (e.g., to determine a recommendation if a respective wellness program 210 has an incorrect attribute in a set of attributes 216 assigned to a respective coach). However, the present disclosure is not limited thereto.

Once a method for measuring “similarity” or “dissimilarity” between points in a dataset has been selected, clustering makes use of a criterion function that measures the clustering quality of any partition of the data. Partitions of the dataset that extremize the criterion function are used to cluster the data. See page 217 of Duda 1973. Criterion functions are discussed in Section 6.8 of Duda 1973. More recently, Duda et al., Pattern Classification, 2nd edition, John Wiley & Sons, Inc. New York, has been published. Pages 537-563 describe clustering in detail. More information on clustering techniques suitable for use as classifiers are disclosed in Kaufman and Rousseeuw, 1990, Finding Groups in Data: An Introduction to Cluster Analysis, Wiley, New York, N.Y.; Everitt, 1993, Cluster analysis (3d ed.), Wiley, New York, N.Y.; and Backer, 1995, Computer-Assisted Reasoning in Cluster Analysis, Prentice Hall, Upper Saddle River, N.J. Particular exemplary clustering techniques that can be used as classifiers include, but are not limited to, hierarchical clustering (agglomerative clustering using nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering.

In some embodiments, a computational model 222 is a nearest neighbor algorithm. For nearest neighbors, given a query point x₀ (a test subject), the k training points x₍ᵣ₎, r, . . . , k (here the training subjects) closest in distance to x₀ are identified and then the point x₀ is classified using the k nearest neighbors. Here, the distance to these neighbors is a function of the abundance values of the discriminating gene set. In some embodiments, Euclidean distance in feature space is used to determine distance as d₍ᵢ₎=∥x₍ᵢ₎−x₍₀₎∥. Typically, when the nearest neighbor algorithm is used, the abundance data used to compute the linear discriminant is standardized to have mean zero and variance 1. The nearest neighbor rule can be refined to address issues of unequal class priors, differential misclassification costs, and feature selection. Many of these refinements involve some form of weighted voting for the neighbors. For more information on nearest neighbor analysis, see Duda, 2001, “Pattern Classification,” Second Edition, John Wiley & Sons, Inc; and Hastie, 2001, “The Elements of Statistical Learning,” Springer, New York, each of which is hereby incorporated by reference in its entirety.

One of skill in the art will readily appreciate that other computational models 222 that are applicable to the systems and methods of the present disclosure. In some embodiments, the systems and methods of the present disclosure utilize more than one computational model to provide a result (e.g., arrive at an evaluation given one or more inputs) with an increased accuracy. For instance, in some embodiments, each respective computational model arrives at a corresponding result when provided a respective data set. Accordingly, in some such embodiments, each respective computational model independently arrives at a result and then the result of each respective computational model is collectively verified through a comparison or amalgamation of the computational models. From this, a cumulative result is provided by the computational models. However, the present disclosure is not limited thereto.

In some embodiments, a respective computational model 222 is tasked with performing a corresponding activity (e.g., step within method 400 of FIGS. 4A through 4E, etc.). As a non-limiting example, in some embodiments, the task performed by the respective computational model includes evaluating a request for a wellness program (e.g., block 404 of FIG. 4A), obtaining a plurality of coaching profiles (e.g., user profiles 208 associated with a respective coach) (e.g., block 410 of FIG. 4B), further obtaining a plurality of wellness programs (e.g., block 422 of FIG. 4C), processing the plurality of coaching profiles, processing the plurality of wellness programs, processing the set of attributes assigned to a respective client (e.g., block 424 of FIG. 4D), considering each respective result provided by a corresponding computational model (e.g., a first computational model 222-1 evaluates a respective result provided by a second computational model 222-2) (e.g., block 436 of FIG. 4E), communicating and/or determining a set of at least one coaching profile and at least one wellness program (e.g., block 442 of FIG. 4E), matching a first client with a coach (e.g., block 488 of FIG. 4E), or a combination thereof. However, the present disclosure is not limited thereto. By way of example, in some embodiments, a second computational model is tasked with generating a respective challenge 26 for a user and/or determining if the respective challenge 26 is deemed complete by the user. In some embodiments, each respective computational model of the present disclosure makes use of 10 or more parameters, 100 or more parameters, 1,000 or more parameters, 10,000 or more parameters, or 100,000 or more parameters. In some embodiments, each respective computational model of the present disclosure cannot be mentally performed.

A wellness system 200 includes a corpus library 224 that is configured to store a plurality of communications corpora 226 (e.g., first communications corpus 226-1, second communications corpus 226-2, . . . , communications corpus W 226-W, etc.). Each communications corpus 226 in the plurality of communication corpora is associated with a unique respective subject matter (e.g., a unique user, a unique medical condition, a unique wellness program, etc.). For instance, in some embodiments, information of the same subject matter (e.g., title, descriptions, goals, and/or corresponding attributes, etc.) is matched to one or more predetermined subject matter using the plurality of computational models 222, such as cosine similarity with term frequency-inverse document frequency (TF-IDF) embedding and transform applied to all texts associated with the information of the same subject matter. In some embodiments, Inter-type matching (e.g., goal-to-description) mostly means checking if a short text is presented (e.g., in slightly changed view) in the larger one. In some embodiments, lemmatization and bi/trigram search approaches are appropriate. As a non-limiting example, in some embodiments, a first communications corpus 226-1 is associated with knee injury medical conditions, a second communications corpus 226-2 is associated with customer support, a third communications corpus 226-3 is associated with social media, a fourth communications corpus 226-4 is associated with politics, a fifth communications corpus 226-5 is associated with comedy, and the like. In some embodiments, these communications corpora are processed by a respective computational model 222 as a training data set, such as including corpus data from medical forums, customer support forums and general social forums where people talk about humor, politics, and general topics in order to detect noise in the communications between clients and coaches through the wellness system 200. However, the present disclosure is not limited thereto.

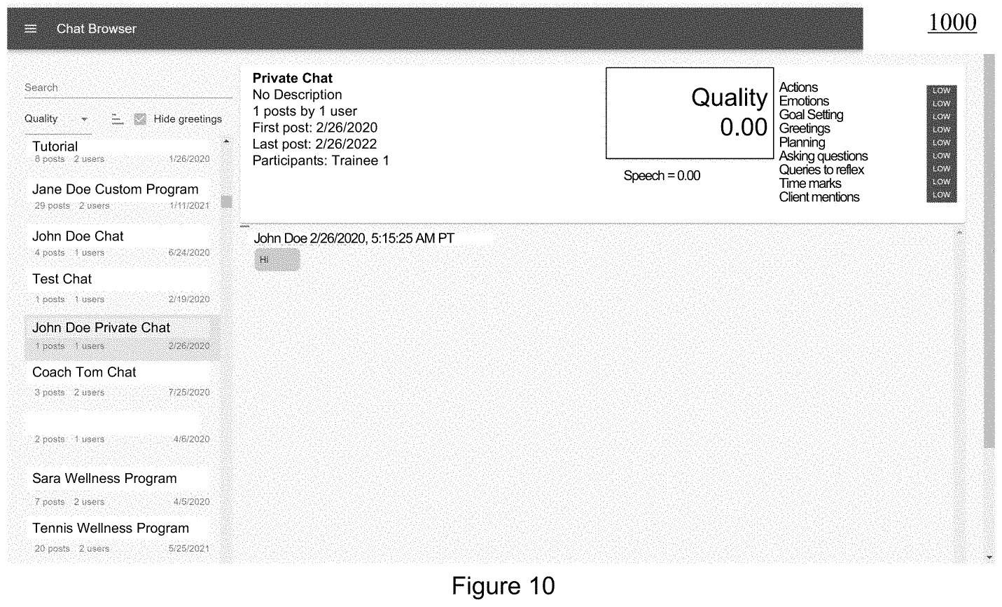

For instance, in some embodiments, a respective communications corpus 226 includes one or more publications (e.g., public communications), such as a scholarly research journal article. As a non-limiting example, clients and coaches are both provided with science-based and historical data sets 212, 218 in order for the wellness system to provide proven recommendations on selection of a set of coaches and wellness programs 210 for a respective client and improve the skills of the coaches. Moreover, these publications allow the wellness system to generate a multi-disciplinary approach to wellbeing and/or treatment of health concerns of a respective client and/or a respective coach based on the scientific research retained in the respective communications corpus. Accordingly, in some embodiments, by using the plurality of computational models 222, one or more connections are obtained between different attributes 216 of the plurality of wellness programs 210 in order to provide a coach with one or more recommendations on related attributes 216 that are effective in improving a respective wellness program 210, such as a depth of description of the respective wellness program (e.g., description checker of FIG. 10), relevance of textual descriptions of the respective wellness program 210, relevance of attributes assigned by the coach for the respective wellness program 210, or a combination thereof. As another non-limiting example, in some embodiments, the publications allow for a respective coach to utilize coaching terms and attributes that are connected to medical conditions and treatments by way of scientific publications retained by the communications corpus 226. From this, the wellness system 200 can determine a projected accuracy and/or precision of the respective wellness program 210 for a particular client based on research and clinical trials of the communications corpus 226, which is further processed by the plurality of computational models. Moreover, a relevance between the one or more wellness programs 210 produced by a coach, especially around proximity and relationship of certain attributes, such as semantic graphs, a relationship in attributes 216 to the scientific concepts and functions provided in the publications, expected outcomes based on best coaching practices provided in the publications (e.g., those that require multi-disciplinary focus), or a combination thereof is determined by the plurality of computational models 222.

Accordingly, in some such embodiments, the wellness system 200 polls for the one or more publications from a remote database. When a determination has been made that a respective publication in the one or more publications exists, the wellness system 200 retrieves the respective publication for storage in a corresponding communications corpus 266. As a non-limiting example, consider the wellness system 200 polling one or more remote devices for a publication associated with tennis. The wellness system 200 polls for a publication, such as when an event occurs (e.g., results of a tennis tournament) and a publication is published, which is then received by the wellness system 200. However, the present disclosure is not limited thereto. In some embodiments, the polling occurs by communicating with one or more remote databases, such as a first database that includes subject-specific aggregations of information, such as university databases, patient records, etc. In some embodiments, the polling occurs by communicating with an internal site that includes searchable databases for internal publications of one or more sites that is dynamically created, such as a knowledge database on a corporate site. In some embodiments, the polling occurs by communicating with one or more publication sources that includes searchable databases for current and archived publications. In some embodiments, the polling occurs by communicating with one or more service providers, such as a classified listing or social media service provider. In some embodiments, the polling of the first communication 240-1 occurs by communicating with a portal that includes more than one of these other categories in searchable databases. In some embodiments, the polling occurs by communicating with one or more computation models 222, such as a database that includes an internal data component for determining one or more results including a dictionary look-ups computational model 222, and a translator between human languages computational model 222, or the like. Additional details and information regarding the receiving of a communication 240 can be found at Bergman, M., 2001, “White Paper: The Deep Web: Surfacing Hidden Value,” Journal of Electronic Publishing, 7(1), print; Dumbacher et al., 2018, “SABLE: Tools for Web Crawling, Web Scraping, and Text Classification,” Federal Committee on Statistical Methodology Research Conference, print; Yan, Y., 2016, “Text Analysis on SEC Filings (A Course Proposal),” print; Rosenfelder et al., 2017, Bayesian Modeling and Advanced Topics in Optimization (Seminar)—Preprocessing Text Data for Sentiment Analysis in R and Python,” print, each of which is hereby incorporated by reference in its entirety.

As yet another non-limiting example, in some embodiments, the respective communication corpus 226 is associated with one or more communication channels facilitated by the wellness system 200 (e.g., by way of client application 320 of client device 300 of FIG. 3, e.g., communication channels of FIG. 10, communication channels of FIG. 11). For instance, in some embodiments, the one or more communication channels associated with the respective communications corpus 226 is particular to a corresponding coach or a corresponding client. In alternative embodiments, the one or more communication channels associated with the respective communications corpus 226 is associated with a predetermined sentiment, such as a positive sentiment (e.g., as determined by one or more computational models 222). Each communication channel enables one-to-one communication or one-to-many communication between a first user and a respective user in a plurality of users and facilitates electronic communication between the first user and the respective user. In some embodiments, a respective communication channel is associated with a social media platform, such as an instant message feature of the social media platform.

In some embodiments, the plurality communications corpus 222 includes at least 5 communications corpora, at least 10 communications corpora, at least 25 communications corpora, at least 50 communications corpora, at least 100 communications corpora, at least 150 communications corpora, at least 250 communications corpora, at least 600 communications corpora, at least 1,000 communications corpora, at least 2,500 communications corpora, at least 5,000 communications corpora, at least 10,000 communications corpora, at least 100,000 communications corpora, or a combination thereof.

Each of the above identified modules and applications correspond to a set of executable instructions for performing one or more functions described above and the methods described in the present disclosure (e.g., the computer-implemented methods and other information processing methods described herein; method 400 of FIGS. 4A through 4E; etc.). These modules (e.g., sets of instructions) need not be implemented as separate software programs, procedures or modules, and thus various subsets of these modules are, optionally, combined or otherwise re-arranged in various embodiments of the present disclosure. In some embodiments, the memory 292 optionally stores a subset of the modules and data structures identified above. Furthermore, in some embodiments, the memory 292 stores additional modules and data structures not described above.

It should be appreciated that the wellness system 200 of FIGS. 2A and 2B is only one example of a wellness system 200, and that the wellness system 200 optionally has more or fewer components than shown, optionally combines two or more components, or optionally has a different configuration or arrangement of the components. The various components shown in FIGS. 2A and 2B are implemented in hardware, software, firmware, or a combination thereof, including one or more signal processing and/or application specific integrated circuits.

Referring to FIG. 3, an exemplary client device 300 is provided (e.g., first client device 300-1). A client device 300 includes one or more processing units (CPUs) 372, one or more network or other communication interfaces 374, memory 392 (e.g., random access memory and/or non-volatile memory) optionally accessed by one or more controllers, and one or more communication busses 374 interconnecting the aforementioned components.

In some embodiments, a client device 300 includes a mobile device, such as a mobile phone, a tablet, a laptop computer, a wearable device such as a smart watch, and the like. In some embodiments, the client device 300 is a desktop computer or other similar devices. In some embodiments, the client device 300 is a standalone device that is dedicated to hosting the plurality of wellness programs 210 of the systems and methods of the present disclosure. Further, in some embodiments, each client device 300 enables a respective user to provide information related to the respective user and/or a different user (e.g., subject preferences, subject feedback, etc.).

In addition, the client device 300 includes a user interface 376. The user interface 376 typically includes a display device 378 for presenting media, such as an evaluation of a respective wellness program 210 or a respective coach and receiving instructions from the subject operating the client device 300. In some embodiments, the display device 378 is optionally integrated within the client device 300 (e.g., housed in the same chassis as the CPU 372 and memory 392), such as a smart (e.g., smart phone) device. In some embodiments, the client device 300 includes one or more input device(s) 380, which allow the subject to interact with the client device 300. In some embodiments, input devices 380 include a keyboard, a mouse, and/or other input mechanisms. Alternatively, or in addition, in some embodiments, the display device 378 includes a touch-sensitive surface, e.g., where display 3778 is a touch-sensitive display or client device 300 includes a touch pad.

In some embodiments, the client device 300 includes an input/output (I/O) subsystem 330 for interfacing with one or more peripheral devices with the client device 300. For instance, in some embodiments, audio is presented through an external device (e.g., speakers, headphones, etc.) that receives audio information from the client device 300 and/or a remote device (e.g., wellness system 200), and presents audio data based on this audio information. In some embodiments, the input/output (I/O) subsystem 330 also includes, or interfaces with, an audio output device, such as speakers or an audio output for connecting with speakers, earphones, or headphones. In some embodiments, the input/output (I/O) subsystem 330 also includes voice recognition capabilities (e.g., to supplement or replace an input device 310).

In some embodiments, the client device 300 also includes one or more sensors (e.g., an accelerometer, an optical sensor, an intensity sensor, a magnetometer, a proximity sensor, a gyroscope, etc.), an image capture device (e.g., a camera device or an image capture module and related components), a location module (e.g., a Global Positioning System (GPS) receiver or other navigation or geolocation system module/device and related components), or a combination thereof. In some embodiments, the one or more sensors of the client device 300 is configured to capture one or more physiological measurements associated with the user, such as a glucose sensor, a heart rate monitor sensor, a blood pressure sensor, a temperature sensor (e.g., a core temperature sensor, a temporal temperature sensor), and the like. In some embodiments, the one or more sensors of the client device 300 include one or more optical sensors. In some embodiments, the one or more optical sensor include a charge-coupled device (CCD) or a one or more complementary metal-oxide semiconductor (CMOS) phototransistors. In some embodiments, the one or more optical sensors receives light from the environment, projected through one or more lens of the client device 300, and converts the light to data representing an image. In some embodiments, the optical sensor captures still images and or video. In some embodiments, a first optical sensor is disposed on a back end portion of the client (e.g., opposite the display on a front end portion of the client device 300), such as to enable the client device 300 for use as a viewfinder for still and or video image acquisition. In some embodiments, a second optical sensor is located on the front end portion of the client device 300 so that an image of the subject is obtained (e.g., to verify the health or condition of the subject, to determine the physical activity level of the subject, to help diagnose a subject's condition remotely, or to acquire visual physiological measurements of the subject, etc.). In some embodiments, a communication channel provided by the client device includes the image and or video captured by the optical sensor (e.g., the communication channel includes a video feed or an image). However, the present disclosure is not limited thereto.

As described above, the client device 300 includes a user interface 376. The user interface 376 typically includes a display device 378, which is optionally integrated within the client device 300 (e.g., housed in the same chassis as the CPU and memory, such as with a smart phone or an all-in-one desktop computer client device 300). In some embodiments, the client device 300 includes a plurality of input device(s) 380, such as a keyboard, a mouse, and/or other input buttons (e.g., one or more sliders, one or more joysticks, one or more radio buttons, etc.). Alternatively, or in addition, in some embodiments, the display device 378 includes a touch-sensitive surface, e.g., where display 308 is a touch-sensitive display 378 or a respective client device 300 includes a touch pad.

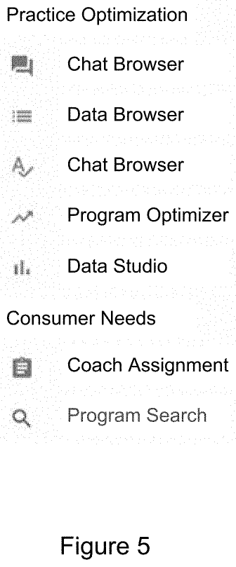

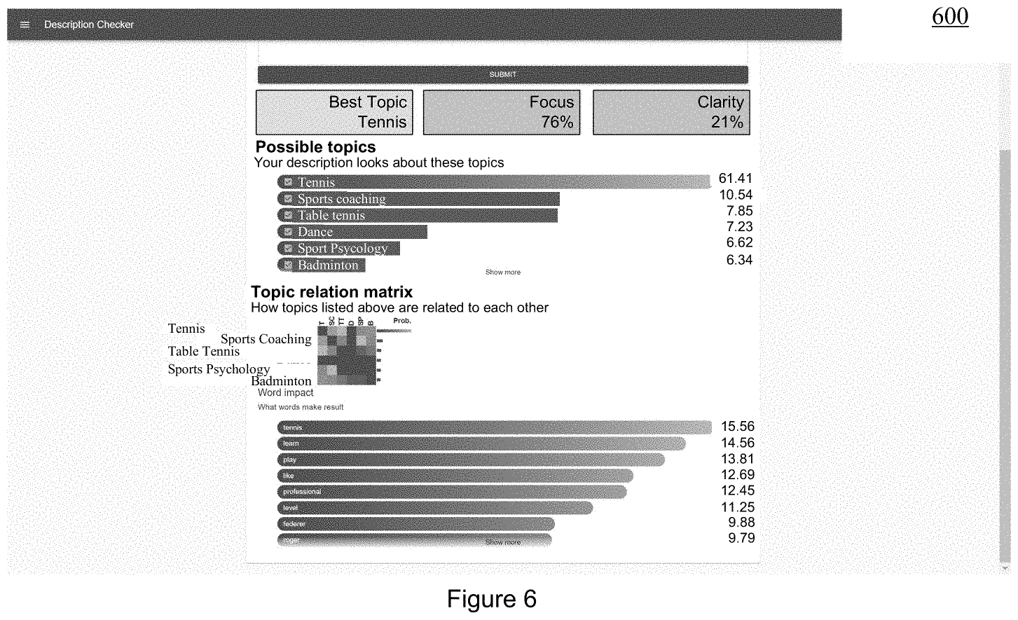

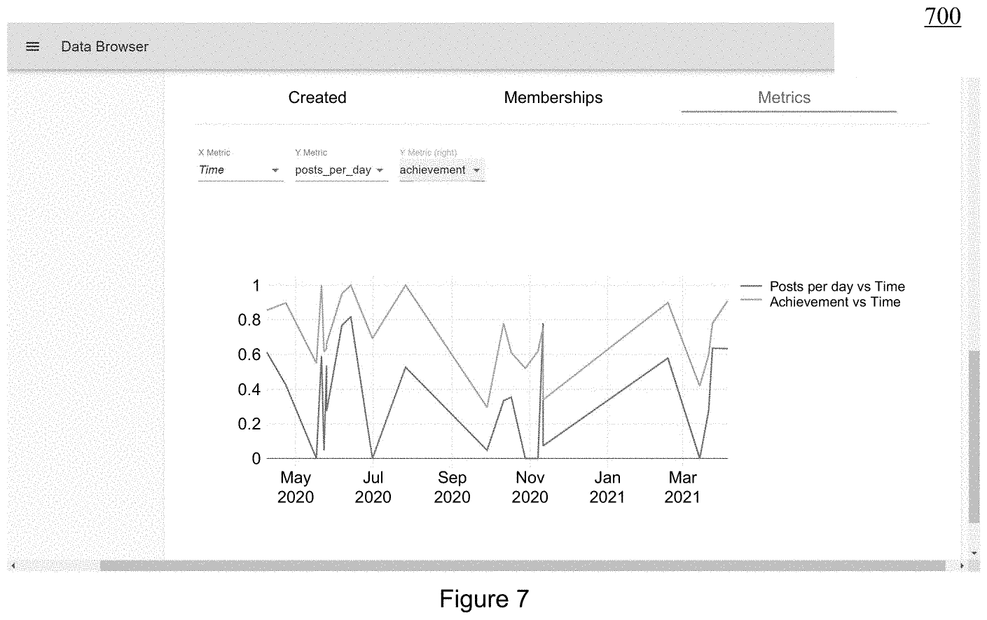

In some embodiments, the client device 300 presents media to a user through the display 378. Examples of media presented by the display 378 include one or more images, a video, audio (e.g., waveforms of an audio sample), or a combination thereof (e.g., user interface 500 of FIG. 5, user interface 600 of FIG. 6, user interface 700 of FIG. 7, user interface. In typical embodiments, the one or more images, the video, the audio, or the combination thereof is presented by the display 378 through a client application 320. In some embodiments, the audio is presented through an external device (e.g., speakers, headphones, etc.) that receives audio information from the client device 300, the wellness system 200, or both, and presents audio data based on this audio information. In some embodiments, the user interface 376 also includes an audio output device, such as speakers or an audio output for connecting with speakers, earphones, or headphones. In some embodiments, the user interface 306 also includes an audio input device (e.g., a microphone), and optional voice recognition capabilities (e.g., to supplement or replace the keyboard). Optionally, the client device 300 includes an audio input device 310 (e.g., a microphone) to capture audio (e.g., speech from a user). In some embodiments, the audio input device 310 is a single omni-directional microphone.

In some embodiments, the client device 300 also includes one or more of: one or more sensors (e.g., accelerometer, magnetometer, proximity sensor, gyroscope); an image capture device (e.g., a camera device or module and related components); and/or a location module (e.g., a Global Positioning System (GPS) receiver or other navigation or geolocation device and related components). In some embodiments, the sensors include one or more hardware devices that detect spatial and motion information about the client device 300. Spatial and motion information can include information about a position of the client device 300, an orientation of the client device 300, a velocity of the client device 300, a rotation of the client device 300, an acceleration of the client device 300, or a combination thereof.

Memory 392 includes high-speed random access memory, such as DRAM, SRAM, DDR RAM, or other random access solid state memory devices, and optionally also includes non-volatile memory, such as one or more magnetic disk storage devices, optical disk storage devices, flash memory devices, or other non-volatile solid state storage devices. Memory 392 may optionally include one or more storage devices remotely located from the CPU(s) 372. Memory 392, or alternatively the non-volatile memory device(s) within memory 392, includes a non-transitory computer readable storage medium. Access to memory 392 by other components of the client device 300, such as the CPU(s) 372 and the I/O subsystem 330, is, optionally, controlled by a controller. In some embodiments, memory 392 can include mass storage that is remotely located with respect to the CPU 372. In other words, some data stored in memory 392 may in fact be hosted on devices that are external to the client device 300, but that can be electronically accessed by the client device 300 over an Internet, intranet, or other form of network 106 or electronic cable using communication interface 304.

In some embodiments, the memory 392 of the client device 300 stores:
an operating system 316 that includes procedures for handling various basic system services;
an electronic address 318 associated with the client device 300 that identifies the client device 300 within a distributed system 100; and
a client application 320 for generating content for display through a graphical user interface presented on the display 308 the client device 300.

An electronic address 318 is associated with the client device 300, which is utilized to at least uniquely identify the client device 300 from other devices and components of the distributed system 100. In some embodiments, the electronic address 318 associated with the client device 300 is used to determine a source of an assessment provided by the client device 300 (e.g., receiving an assessment from the wellness system 200 and communicating one or more responses based on the assessment).

In some embodiments, a client application 320 is a group of instructions that, when executed by a processor, generates content for presentation to the user, such as a communication channel, a video conference, a survey, a search result, and the like. The client application 320 may generate content in response to inputs received from the user through the client device 300, such as the inputs 310 of the client device. As a non-limiting exemplary embodiment, in some embodiments, the client application 320 presents a wellness platform of the wellness system 200. In some such embodiments, the client application 320 provides a user that is either a client or a coach with a common functionality such as sign-up for the wellness platform, login to the wellness platform, password recovery, user profile 208 management, and the like. In some embodiments, the client application 320 allows one or more clients and one or more coaches to participate in a communication channel. For instance, in some embodiments, a first communication channel is one-to-one (e.g., private) between a first coach and a first client. In other embodiments, the first communication channel includes a plurality of participants, such as the first client, a second client, and the first coach. In some embodiments, the client application 320 allows both one or more clients and one or more coaches to participate to participate in a video conference, much like aforementioned communication channel. In some embodiments, the client application 320 allows a coach to create a respective wellness program 210, edit the respective wellness program 210, lead the respective wellness program 210 (e.g., engage with one or more clients associated with the respective wellness program 210), delete the respective wellness program 210, manage financials of the respective wellness program 210, obtain statistical data sets (e.g., by computational models 222 of wellness system 200 of FIG. 2), or a combination thereof. Moreover, in some embodiments, the client application 320 provides a survey to a user, such as a client. Furthermore, in some embodiments, the client application 320 allows a user to search for the respective wellness program 210, engage with the respective wellness program 210 (e.g., purchase and/or participate in the respective wellness program 210), complete the respective wellness program 210, declare one or more goals of the user to complete during the respective wellness program 210 or when completing the respective wellness program 210, provide achievements completed by the user over a period of time, provide feedback for the respective wellness program 210 and/or the coach associated with the respective wellness program 210, or a combination thereof.

Each of the above identified modules and applications correspond to a set of executable instructions for performing one or more functions described above and the methods described in the present disclosure (e.g., the computer-implemented methods and other information processing methods described herein, method 400 of FIGS. 4A through 4E, etc.). These modules (e.g., sets of instructions) need not be implemented as separate software programs, procedures or modules, and thus various subsets of these modules are, optionally, combined or otherwise re-arranged in various embodiments of the present disclosure. In some embodiments, the memory 392 optionally stores a subset of the modules and data structures identified above. Furthermore, in some embodiments, the memory 392 stores additional modules and data structures not described above.

It should be appreciated that the client device 300 of FIG. 3 is only one example of a client device 300, and that the client device 300 optionally has more or fewer components than shown, optionally combines two or more components, or optionally has a different configuration or arrangement of the components. The various components shown in FIG. 3 are implemented in hardware, software, firmware, or a combination thereof, including one or more signal processing and/or application specific integrated circuits.

Now that a general topology of the distributed system 100 has been described in accordance with various embodiments of the present disclosures, details regarding some processes in accordance with FIGS. 4A through 4E will be described. FIGS. 4A through 4E illustrates a flow chart of methods (e.g., method 400) for matching a first client and a coach, such as by providing a set of at least one coaching profile and at least one wellness program that is associated with the coach, in accordance with embodiments of the present disclosure. Specifically, an exemplary method 400 for matching the first client and the coach is provided, in accordance with some embodiments of the present disclosure. In the flow charts, the preferred parts of the methods are shown in solid line boxes, whereas optional variants of the methods, or optional equipment used by the methods, are shown in dashed line boxes.

Various modules in the memory 292 of the wellness system 200, the memory 392 of a client device 300, or both perform certain processes of the methods 400 described in FIGS. 4a through 4E, unless expressly stated otherwise. Furthermore, it will be appreciated that the processes in FIGS. 4A through 4E can be encoded in a single module or any combination of modules.

Block 402. Referring to block 402 of FIG. 4A, a method 400 of matching a first client (e.g., a first user of a first client device 300-1) and a coach (e.g., a second user of a second client device 300-2) at (using) a computer system (e.g., wellness system 200). The computer system 200 includes one or more processors (e.g., CPU 274 of FIGS. 2A and 2B) and a memory (e.g., memory 292 of FIGS. 2A and 2B) coupled to the one or more processors 274. The memory 292 includes at least one programs (e.g., user library 206 of FIG. 2A, wellness program library 214 of FIG. 2B, computational model library 220 of FIG. 2B, client application 320 of FIG. 3, etc.) configured to be executed by the one or more processors 274.

In some embodiments, prior to performing the method 400, the first client and the coach are unknown to one another (e.g., the first client has never engaged with the coach or a respective wellness program 210 associated with the coach). Accordingly, the method 400 brings together the first client and the coach when such a connection would otherwise never come to fruition without random encounter.

Block 404. Referring to block 404, the method 400 includes receiving a request for a wellness program (e.g., wellness program 210 of FIGS. 2A and 2B). The request for the wellness program 210 is received in electronic form, such as by way of a communication network 106. In some embodiments, the request is in the form of an application programming interface (API) call to the wellness system 200 from a client device 300 associated with the client. In some embodiments, the request for the wellness program 210 is a survey completed by the client at a client device 300. The survey includes a plurality of questions or prompts that is configured to elicit a corresponding plurality of responses from the client, which form a basis of the request for the wellness program.

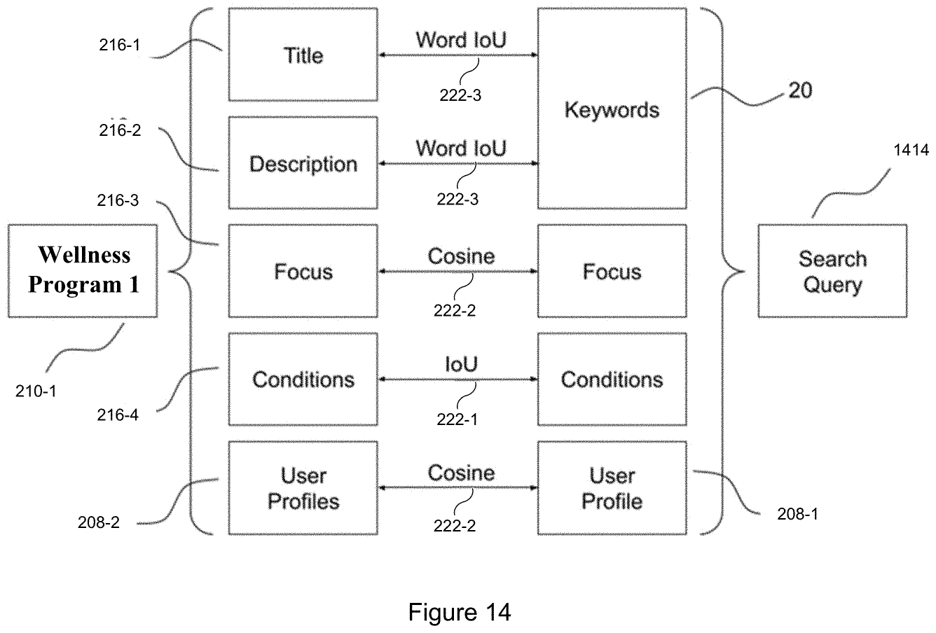

The request includes a set of attributes (e.g., attribute(s) 216 of FIG. 2B) that is assigned to the first client. In some embodiments, this set of attributes 216 is assigned from a plurality of attributes 216. In some embodiments, the plurality of attributes is an aggregation of each unique attribute 216 associated with a respective wellness program 210 of the wellness system 200. For instance, referring briefly to FIG. 14, in some embodiments, the set of attributes assigned to each client collectively include a focus of a respective wellness program 210, one or more conditions associated with the respective wellness program 210 and/or the client, one or more personal characteristics associated with the client, or a combination thereof. However, the present disclosure is not limited thereto.

In some embodiments, the set of attributes 216 includes at least 1 attribute, at least 2 attributes, at least 5 attributes, at least 10 attributes, at least 25 attributes, at least 50 attributes, at least 100 attributes, at least 150 attributes, at least 250 attributes, at least 600 attributes, at least 1,000 attributes, at least 2,500 attributes, at least 5,000 attributes, at least 10,000 attributes, or a combination thereof.

As another non-limiting example, in some embodiments, the plurality of attributes 216 is obtained from the survey completed by the client, such as by providing one or more responses from the survey to a plurality of computational models 226. For instance, after registering with the wellness system 200 and creating a corresponding user profile 208 (e.g., third user profile 208-3) associated with the client, the client is presented with the survey through a display (e.g., display 378 of FIG. 3) of the client device. After completing some or all of the survey, the corresponding plurality of responses provided by the client is received by the wellness system 200. Accordingly, using one or more computational models 222 (e.g., two computational models 222), the set of attributes is obtained, such as a first medical condition of the client (e.g., recent tear in muscle) associated with a first attribute in the set of attributes 216 and a first health-related concern of the client (e.g., old age) associated with a second attribute 216-2 in the set of attributes 216.

Blocks 406-408. Referring to blocks 406 and 408, in some embodiments, the set of attributes 216 includes one or more focus attributes 216 and one or more personal attribute 216, such as medical information and desired disciplines of the wellness program 210. More particularly, in some embodiments, the set of attributes 216 includes one or more medical attributes associated with the first client. In some embodiments, the set of attributes 216 includes one or more temporal attributes associated with completing the respective wellness program, such as a period of time consumed completing the respective wellness program 210. In some embodiments, the set of attributes 216 includes one or more geographic attributes associated with completing the respective wellness program 210, such as a preferred geographic location of where to conduct the respective wellness program 210. In some embodiments, the set of attributes 216 includes one or more accounting attributes 216 associated with the respective wellness program, which is associated with a financial aspect of providing and/or engaging with the respective wellness program 210. In some embodiments, the one or more accounting attributes 216 includes a price of the respective wellness program 210 (e.g., $50 per session of a first wellness program 210-1, $1,000 for the entire second wellness program 210-2, etc.), a schedule of the respective wellness program 210 (e.g., a calendar of when and/or where events for the respective wellness program 210 occur), a plurality of tasks associated with the respective wellness program (e.g., an initial task, one or more intermediate tasks, and a final task for completing the respective wellness program 210), one or more communication conferences associated with the respective wellness program 210 (e.g., required communications in a communication channel with a coach of the respective wellness program), one or more quantitative goals associated with the respective wellness program 210, or a combination thereof. In some embodiments, the set of attributes 216 includes one or more physical attributes associated with the respective wellness program, such as a desired weight loss from completing the respective wellness program 210 (e.g., lose 2% body fat of a respective client) or a desired strength gain (e.g., gain 5% upper strength). In some embodiments, the set of attributes 216 includes one or more mental attributes 216 associated with the respective wellness program.

Block 410. Referring to block 410 of FIG. 4, the method 400 includes obtaining a plurality of coaching profiles, which is a set of user profiles 208 from a user library that is associated with coaches of the wellness platform, as opposed to clients. This obtaining of the plurality of coaching profiles is in response to receiving the request for the wellness program 210, which allows for the plurality of coaching profiles obtained from the user library 206 to be tailored to the request, ensuring the client is matched with the best coach. As described above, each coaching profile in the plurality of coaching profiles is associated with a corresponding coach, which ensures that only a respective user profile 208 associated with a coach is obtained responsive to the request.

In some embodiments, the plurality of coaching profiles includes at least 2 coaching profiles, at least 5 coaching profiles, at least 10 coaching profiles, at least 25 coaching profiles, at least 50 coaching profiles, at least 100 coaching profiles, at least 150 coaching profiles, at least 250 coaching profiles, at least 600 coaching profiles, at least 1,000 coaching profiles, at least 2,500 coaching profiles, at least 5,000 coaching profiles, at least 10,000 coaching profiles, at least 100,000 coaching profiles, or a combination thereof.

Furthermore, each coaching profile includes a corresponding one or more wellness programs (e.g., second user profile 208 that is associated with a coach is further associated with first wellness program 210-1 and third wellness program 210-3). In some embodiments, each wellness program 210 in the corresponding one or more wellness programs 210 associated with the coaching profile is authored, at least in part, by the corresponding coach, such as by using the input 380 of the client device associated with the corresponding user profile 208. As described supra, allowing the coach to create and edit the wellness programs 210 allows for each coach to optimize engagement with a respective client according to the expertise and experience of the coach.

Furthermore, each coaching profile includes a first corresponding data set associated with a corresponding first historical performance of the corresponding coach (e.g., user historical performance 212-1 associated with a corresponding user profile 208-1). In some embodiments, the corresponding first historical performance 212-1 of the corresponding coach includes data elements that are captured and/or derived from when the coach is engaging with a respective wellness program 210 in the corresponding one or more wellness program 210.

Block 412. Referring to block 412, in some embodiments, the corresponding first historical data set 212-1 for a corresponding coach in the plurality of coaching profiles includes a universal success rate for the one or more wellness programs 210 associated with the corresponding coach, an individual success rate for each wellness program 210 in the corresponding one or more wellness programs 210, a universal enrollment rate for the corresponding one or more wellness programs 210, an individual enrollment rate for each wellness program 210 in the corresponding one or more wellness programs 210, a communications corpus 226 associated with the corresponding coach (e.g., some or all of a respective communications corpus 226 associated with the coach and/or the one or more wellness programs 210), an engagement rate for each wellness program 210 in the corresponding one or more wellness programs 210, a frequency rate for each wellness program 210 in the corresponding one or more wellness programs 210, a frequency rate for each wellness program 210 in the corresponding one or more wellness programs 210, the data set associated with the corresponding one or more wellness programs 210, or a combination thereof.

Blocks 414-416. Referring to blocks 414 and 416, in some embodiments, the corresponding first historical data set 212-1 includes (e.g., references) the communications corpus 226 that is associated with the corresponding coach. In some embodiments, the communications corpus 226 includes a record of a corresponding plurality of messages for each communication channel in a plurality of communication channels associated with the corresponding coach. By including the record of the corresponding plurality of messages, the corresponding first historical data set 212-1 includes references points from each communication channel the coach engages with, which allows for the method 400 to evaluate how the engage engages with each communication channel.

In some embodiments, by having the communications corpus 212-2 included in the corresponding first historical data set 212-1, the method 400 provides content analysis for prior communication channels associated with the coach. For instance, in some embodiments, a presence of medical terms stated by the coach is checked by the plurality of computational models to prove that a conversation with a respective client is not off-topic to the wellness program.

In some embodiments, this including of the communications corpus allows for the method to ensure that a respective coach matched for the client ensures a threshold level of non-abusive, positive sentiment, and polite language when engaging through a communication channel.

Block 416. Referring to block 416, in some embodiments, each communication channel in the plurality of communication channels facilitates an exchange of a plurality of messages between the corresponding coach and a respective subject in a plurality of subjects. For instance, as described supra, in some embodiments, the exchange of the plurality of messages is by text (e.g., communication channels of FIG. 10, communication channels of FIG. 11, etc.). In some embodiments, the exchange of the plurality of messages is by video or audio. Furthermore, in some embodiments, the respective subject is the client. In some embodiments, the respective subject is a respective client associated with the coach, such as a first client engaging with a first wellness program 210-1 further associated with the coach. However, the present disclosure is not limited thereto.

Block 418. Referring to block 418, in some embodiments, the communications corpus 226 provides a temporal ordering to each message in the record of the corresponding plurality of exchange of the plurality of messages, such as an order from oldest message to newest message. In this way, in some embodiments, the communications corpus 226 gives a certain weight to one or more messages depending on a placement of the one or more messages within the temporal ordering. For instance, in some embodiments, messages that are older in the temporal ordering are given a lower weight to give emphasis on building new communication techniques for the coach or a higher weight to give emphasis on building established communication techniques for the coach.

Block 420. Referring to block 420, in some embodiments, the respective wellness program 210 is a recreational activity and/or a sport activity. In some embodiments, the recreation activity includes those activities that the client chooses to do to refresh his or her body and/or minds and make free time more interesting and enjoyable for the client. Examples of recreation activities are walking, music, non-competitive swimming, meditation, reading, playing games (e.g., board games, physical games such as tetherball, card games, mental games, etc.), and dancing. Moreover, in some embodiments, the sport activity refers to any type of organized physical activity, such as soccer, rugby, football, basketball, and athletics (e.g., gymnastics, track and field, etc.). As a recreational activity and/or a sport activity, the respective wellness program 210 is improves the quality of performance of the client by having the client perform the recreational activity and/or the sport activity with the coach that is accomplished in the activity.

Block 422. Referring to block 422 of FIG. 4C, the method 400 includes further obtaining a plurality of wellness programs 210, such as from the wellness program library 214 of the wellness system 200. Each respective wellness program 210 in the plurality of wellness program 210 is associated with one or more corresponding coaches in the plurality of coaches, such as the one or more corresponding coaches that authored the respective wellness program 210. In some embodiments, the one or more corresponding coaches includes a primary coach that is responsible for leading a respective wellness program 210 and at least one supporting coach that helps the primary coach lead the respective wellness program 210.

In some embodiments, the plurality of wellness programs 210 includes at least 2 wellness programs, at least 5 wellness programs, at least 10 wellness programs, at least 25 wellness programs, at least 50 wellness programs, at least 100 wellness programs, at least 150 wellness programs, at least 250 wellness programs, at least 600 wellness programs, at least 1,000 wellness programs, at least 2,500 wellness programs, at least 5,000 wellness programs, at least 10,000 wellness programs, at least 100,000 wellness programs, or a combination thereof.

Furthermore, each respective wellness program 210 includes one or more attributes 216 that is improved for the client by the respective wellness program, such as when completing the respective wellness program and/or when engaging with the respective wellness program. In some embodiments, a respective attribute 2116 in the one or more attribute is assigned to the coach when the respective wellness program 210 is created by the coach. In some embodiments, the respective attribute 2116 in the one or more attribute is assigned to a client who has completed the respective wellness program 210, which allows for providing feedback by way of the attributes 216, such as if the coach provides an inaccurate attribute 216 when creating the respective wellness program 210.

Additionally, each respective wellness program 210 includes and a second corresponding data set associated with a second historical performance of the respective wellness program during the respective wellness program (e.g., wellness program historical performance 218 of FIG. 2B). In some embodiments, this second corresponding data set includes object data associated with the second historical performance, such as a projection of a future return on invention for a coach and/or a client that completes the respective wellness program 210. As described supra, the wellness program historical performance 218 of the second corresponding data set allows for the method to evaluation past performances by a respective client and a respective coach of the respective wellness program 210. In some embodiments, the second historical performance is a completed performance of the respective wellness program 210 by the respective client, which ensures that only those clients that have completed the respective wellness program 210 are included in the wellness program historical performance 218. However, the present disclosure is not limited thereto.

Block 424. Referring to block 424 of FIG. 4D, the method 400 includes processing the plurality of coaching profiles (e.g., block 410 of FIG. 4B), the plurality of wellness programs (e.g., block 422 of FIG. 4C), and the set of attributes assigned to the first client (e.g., block 404 of FIG. 4A). This processing is performed by a plurality of computational models 222, such as about three computational models 222. In some embodiments, the plurality of computations 222 produce a respective result for each computational model 222 in the plurality of computational models 222, which allow for producing multiple results to achieve higher accuracy and precision. Each respective result provided by the computational model 222 is a data set that is associated with the one or more wellness programs 210.

Block 426. Referring to block 426, in some embodiments, a respective computational model in the plurality of computational models 222 includes determining, for each respective sentiment in a plurality of sentiments, whether a corresponding sentiment analysis criterion is satisfied or not satisfied by taking a cosine similarly measure or dot product of one or more data elements in the corresponding coaching profile against each reference statement in a corresponding list of reference statements for the respective sentiment that are deemed to be attributive of a predetermined sentiment.

In some embodiments, the plurality of sentiments includes a positive sentiment, a neutral sentiment, a negative sentiment, or a combination thereof. Moreover, in some embodiments, the sentiment of the data construct includes a combination or two or more sentiments (e.g., a positive sentiment and a negative sentiment combine to form a neutral sentiment, a positive sentiment and a negative sentiment combine to a sentiment that is weighted towards one of the positive sentiment or the negative sentiment, etc.).

In some embodiments, the plurality of computational models 222 include a lexical based computational model such as a corpus based computational model (e.g., semantic based or statistical based) or a dictionary based computational model. In some embodiments, the plurality of computational models 222 includes a plurality of one or more correlation models, one or more comparison models, one or more regression models, one or more computational models 222, one or more survival analysis models, one or more product limit estimation models, one or more ranking models, one or more cox proportional hazard models, or a combination thereof. In some embodiments, the plurality of computational models 222 includes one or more random forest models, one or more random survival forest models, one or more extreme gradient boosting models, one or more support vector machine models, one or more Gaussian mixture models, one or more neural network models, or a combination thereof.

Turning to more specific aspects of using the plurality of computational models 222 to evaluate the sentiment and gauge, predict, maintain, or a combination thereof high levels of engagement for the client and/or the coach with a respective wellness program 210.

A decision tree computational model 222 is a supervised learning classification model that solves various regression and classification problems. The decision tree computational model 222 using one or more branching nodes associated with an attribute (e.g., a source, a text string, etc.) of an input (e.g., data construct) and leaf (e.g., end) nodes associated with a classification label (e.g., a characteristic such as a sentiment, an emotion, etc.). Evaluating and providing a characteristic of a communication using the decision tree computational model 222 includes starting at a root (e.g., base) of a decision tree. An attribute of the root is compared with the communication to evaluate a characteristic. The comparison continues through one or more intermediate (e.g., internal) nodes until a leaf node is reached to provide the characteristic. To select the attribute, in some embodiments, instructions 210 associated with the decision tree computational model 222 include a plurality of information gain (e.g., Gini index) instructions 210. The information gain instructions 210 estimate a distribution of the information included in each attribute. The information gain instructions 210 provide a metric of a degree of elements incorrectly identified. For instance, an information gain I of zero (0) is considered perfect with no errors. To measure an uncertainty of a random variable X (e.g., a text string or an ideogram of a communication), entropy H for a binary classification problem of two classes (e.g., positive sentiment or negative sentiment) is defined as, where p(x) is the proportion of the random variable X, E is the expected value:

H(X)=E_{X}[I(x)]=−Σ(p(x)log p(x)).

In some embodiments, the decision tree computational model 222 is a binary classification having a positive and a negative class (e.g., proportion of binary classification x1 and x2 is −(p(x1) log(px. If the classes of a data construct are all positive or all negative, the entropy is considered zero. If half of the classes are positive and the other half are negative, the entropy is considered to be one (1). Since the Gini Index is a mechanism to determine if a portion of the data construct is incorrectly analyzed, elements having an attribute with a lower relative Gini Index are preferred since a lower relative Gini Index relates to a higher accuracy in evaluating and providing a characteristic of a communication.

In some embodiments, a decision tree computational model 222 has an overfitting problem in accordance with a determination that the decision tree computational model 222 goes deeper and deeper (e.g., higher order series of branches, meaning an increased number of internal nodes). To avoid this overfitting problem, in some embodiments, the instructions 210 of the decision tree computational model 222 includes one or more pre-pruning instructions and/or one or more post-pruning instructions. These pre-pruning instructions and post-pruning instructions 210 reduce a number of branches within the decision tree of the decision tree computational model 222. Furthermore, these pre-pruning instructions and post-pruning instructions 210 allow the decision tree computational model 222 to cease tree growth and cross validate data, increasing an accuracy for evaluating and providing a characteristic of a communication.

Utilizing a decision tree computational model 222 requires less processing time for evaluating and providing a characteristic of a communication. Furthermore, the decision tree computational model 222 is not affected if a non-linear relationship exists between different parameters of the classification evaluation. However, in some embodiments, the decision tree computational model 222 has difficulty handling non-numeric data, and small change in the data (e.g., evolution of a language such as a new slang term) may lead to a major change in the tree structure and logic.

In some embodiments, the neural network computational model 222 includes a convolutional neural network (CNN) and/or a region-convolutional neural network (RCNN). In some embodiments, the neural network computational model 222 includes an inter-pattern distance based (DistAI) computational model 222 (e.g., a constructive neural network learning computational model 222).

In some embodiments, the inter-pattern distance based computational model 222 includes a multi-layer network of threshold logic units (TLU), which provide a framework for pattern (e.g., characteristic) classification. This framework includes a potential to account for various factors including parallelism of data, fault tolerance of data, and noise tolerance of data. Furthermore, this framework provides representational and computational efficiency over disjunctive normal form (DNF) expressions and the decision tree computational model 222. In some embodiments, a TLU implements an (N−1) dimensional hyperplane partitioning an N-dimensional Euclidean pattern space into two regions. In some embodiments, one TLU neural network sufficiently classifies patterns in two classes if the two patterns are linearly separable. Compared to other constructive learning computational model 222, the inter-pattern distance based computational model 222 uses a variant TLU (e.g., a spherical threshold unit) as hidden neurons. Additionally, the distance based computational model 222 determines an inter-pattern distance between each pair of patterns in a training data set (e.g., reference text 214 of FIG. 2), and determines the weight values for the hidden neurons. This approach differs from other computational model 222 that utilize an iterative classification process to determine the weights and thresholds for evaluating and providing a characteristic of a communication.

In some embodiments, the distance based computational model 222 utilizes one or more types of distance metric to determine the inter-pattern distance between each pair of patterns. For instance, in some embodiments, the distance metric is based on those described in Duda et al., 1973, “Pattern Classification and Scene Analysis,” Wiley, Print., and/or that described in Salton et al., 1983, “Introduction to Modern Information Retrieval,” McGraw-Hill Book Co., Print, each of which is hereby incorporated by reference in their entirety. Table 1 provides various types of distance metrics of the distance based computational model 222.

TABLE 1
Exemplary distance metrics for the distance based computational model 222.
Consider X^{p} = [X₁^{p}, . . . , Xₙ^{p}] and X^{q} = [X₁^{p}, . . . , Xₙ^{q}] to be two pattern vectors. Also
consider maxᵢ and minᵢ to be the maximum value and the minimum value of an i^{th} attribute of
the patterns in a data set (e.g., a communication and/or a text string), respectively. The
distance between X^{p} and X^{q} is defined as follows for each distance metric:

| Type | Distance Metric |
|---|---|
| Euclidean | $d ( {X}^{p} , {X}^{q} ) = \sqrt{{\sum }_{i = 1}^{n} {( {X}_{i}^{p} - {X}_{i}^{q} )}^{2}}$ |
| Manhattan | $d ( {X}^{p} , {X}^{q} ) = {\sum }_{i = 1}^{n} \vert {X}_{i}^{p} - {X}_{i}^{q} \vert$ |
| Maximum | d(X^{p}, X^{q}) = argmaxᵢ\|Xᵢ^{p} − Xᵢ^{q}] |
| Value | |
| Normalized Euclidean | $d ( {X}^{p} , {X}^{q} ) = \sqrt{\frac{1}{n} {\sum }_{i = 1}^{n} {( \frac{{X}_{i}^{p} - {X}_{i}^{q}}{{max}_{i} - {min}_{i}} )}^{2}}$ |
| Normalized Manhattan | $d ( {X}^{p} , {X}^{q} ) = \frac{1}{n} {\sum }_{i = 1}^{n} \frac{\vert {X}_{i}^{p} - {X}_{i}^{q} \vert}{{max}_{i} - {min}_{i}}$ |
| Normalized Maximum Value | $d ( {X}^{p} , {X}^{q} ) = {argmax}_{i} \frac{\vert {X}_{i}^{p} - {X}_{i}^{q} \vert}{{max}_{i} - {min}_{i}}$ |
| Dice Coefficient | $d ( {X}^{p} , {X}^{q} ) = 1 - \frac{2 {\sum }_{i - 1}^{n} {X}_{i}^{p} {X}_{i}^{q}}{{\sum }_{i - 1}^{n} {X}_{i}^{{p}^{2}} + {\sum }_{i - 1}^{n} {X}_{i}^{{q}^{2}}}$ |
| Cosine coefficient | $d ( {X}^{p} , {X}^{q} ) = 1 - \frac{{\sum }_{i - 1}^{n} {X}_{i}^{p} {X}_{i}^{q}}{\sqrt{{\sum }_{i - 1}^{n} {X}_{i}^{{p}^{2}} \cdot {\sum }_{i - 1}^{n} {X}_{i}^{{q}^{2}}}}$ |
| Jaccard coefficient | $d ( {X}^{p} , {X}^{q} ) = 1 - \frac{{\sum }_{i - 1}^{n} {X}_{i}^{p} {X}_{i}^{q}}{{\sum }_{i - 1}^{n} {X}_{i}^{{p}^{2}} + {\sum }_{i - 1}^{n} {X}_{i}^{{q}^{2}} - {\sum }_{i - 1}^{n} {X}_{i}^{p} {X}_{i}^{q}}$ |

Additional details and information regarding the distance based computational model 222 can be learned from Yang et al., 1999, “DistAI: An Inter-pattern Distance-based Constructive Learning Algorithm,” Intelligent Data Analysis, 3(1), pg. 55, which is hereby incorporated by reference in its entirety.

Distance based computational model 222 use of one or more spherical threshold neurons in a hidden layer to determine a cluster of patterns for classification by each hidden neuron, allowing the distance based computational model 222 to have a higher accuracy and an improved processing performance in evaluating and providing a characteristic of a communication as compared to other computational models 222. This improved performance holds particularly true for large data sets, such as those provided as reference text 214. If the distance based computational model 222 is trained using reference text 214, a processing time shortens for evaluating and providing a characteristic of a communication to other computational models 222. However, the distance based computational model 222 computational model 222 requires maintenance of an inter-pattern distance matrix during the training of the computational model 222. Additionally, the distance based computational model 222 consumes more memory (e.g., memory 292 of FIG. 2A, memory 392 of FIG. 3, etc.) compared to other computational models 222 due to the large data set sizes associated with the distance based computational model 222.

In some embodiments, the Bayesians Network computational model 222 includes one or more attribute node (e.g., characteristic node), that each lack a parent node except a class node. Furthermore, all attributes are independently given a value of a class variable. The Bayesian theorem provides a mechanism for optimally predicting a class of a previously unseen example data (e.g., a communication provided by a user). A classifier is a function assigning a class label to a communication and/or text string. Generally, a goal of learning computational models 222 is to construct the classifier for a given set of training data (e.g., reference text 214 of FIG. 2) with a class label.

For instance, consider E to represent a sequence element of attribute values (x₁, x₂, . . . , xₙ), where xᵢ is a value of an attribute Xi, consider C to represent a classification variable, and consider c to represent a value of C. If an assumption is made that both positive (+) or negative (−) classes exist (e.g., a positive sentiment and a negative sentiment), a probability of an example data E=(x₁, x₂, . . . , xₙ), being of a class c is:

$$p ( c \vert E ) = \frac{p ( E \vert c ) p ( c )}{p ( E )} .$$

E is classified as the class C=+if and only if

$${f}_{b} ( E ) = \frac{p ( C = + \vert E )}{p ( C = - \vert E )} \geq 1 ,$$

wherein f_{b}(E) is a Bayesian classifier. Furthermore, in some embodiments, if all attributes are assumed independent values of the class variable then p(E|c)=p(x₁, x₂, . . . , xₙ|c)=Åᵢ₌₁ⁿp(xᵢ|c), and a resulting classifier is

$${f}_{nb} ( E ) = \frac{p ( C = + )}{p ( C = - )} = {\prod }_{i = 1}^{n} \frac{p ( {x}_{i} \vert C = + )}{p ( {x}_{i} \vert C = - )} ,$$

with f_{nb}(E) being a Naïve Bayes classifier.

Naïve Bayes computational models 222 are typically simple to implement and have a fast processing time. This improved performance is due in part to Naïve Bayes computational models 222 requiring less extensive set of training data (e.g., reference text 214 of FIG. 2), and performing well with binary (e.g., a positive sentiment or a negative sentiment characteristics) and multi-class (e.g., emotional characteristics) classifications. In some embodiments (e.g., if the Naïve Bayes conditional independent assumptions holds), the Naïve Bayes computational models 222 provides improved performance and accuracy in predicting a characteristic of a communication as compared to a logistic regression discriminative computational model 222. Furthermore, memory (e.g., memory 290 and/or 192 of FIG. 2, memory 307 of FIG. 3, etc.) and CPU (e.g., CPU 274 of FIG. 2, CPU 392 of FIG. 3, etc.) utilizations are modest to run the computational models 222 since the operations are not required to hold the whole data set in the memory of the system 100.

In some embodiments, the Support Vector Machine (SVM) computational model 222 provides classification and/or regression evaluation processes. The SVM computational model 222 is a supervised learning classification model and primarily utilized in classification processes. Generally, a binary computational model 222 is given a pattern x drawn from a domain X. The binary computational model 222 estimates which value an associated binary random variable, considering y E {±1}, will assume. For instance, given pictures of apples and oranges, a user might want to state whether the object in question is an apple or an orange. Equally well, a user might want to predict whether a homeowner might default on his loan, given income data, credit history, or whether a given Email is junk or genuine.

The Support Vector Machine (SVM) computational model 222 performs classification for determining a characteristic of a communication by finding a hyperplane that maximizes a margin between two respective classes of characteristics. Accordingly, the support vector is the vectors that define the hyperplane. In other words, SVM classification is the partition that segregates the classes. A vector is an object that has both a magnitude and a direction. In geometrical term, a hyperplane is a subspace whose dimension is one less than that of its ambient space. If a space is in 3-dimensions then a hyperplane is a plane, if a space is in 2-dimensions then a hyperplane is a line, if a space in one dimension then a hyperplane is a point, and the like.

Often, a communication includes objective language, which typically is unbiased and not influenced by opinion, and/or subjective language, which express opinion and judgement. Accordingly, in some embodiments, parsing the communication to determine objective language within the communication and/or subject language within the text box is useful to increase accuracy of determining a characteristic of the communication. Thus, in some embodiments, facts and information are evaluated from the objective language of the communication, and sentiment and/or emotion are evaluated from the subjective language of the communication.

In some embodiments, parsing the communication into one or more text strings further includes applying a pattern matching computational model 222, such as a keyword analysis classification model and/or a simple parsing classification model. In some embodiments, the pattern matching computational model 222 including parsing the communication on a sentence-by-sentence basis or a word-by-word basis to determine a part-of-speech (e.g., a clause, a verb, a noun, a pronoun, an adverb, a preposition, a conjunction, an adjective, an interjection, etc.) of the portion of the communication. Knowing the part-of-speech of the portion of the communication provides a grammatical description of the portion of the text, aiding in the evaluating and providing of a characteristic of a communication.

Furthermore, knowing the part-of-speech of the portion of the communication allows for the system 100 to exclude trivial portions of the communication while evaluating and providing the characteristic of the communication, improving processing performance of the system 100. In some embodiments, the trivial portions of the communication include an article, a preposition, a conjunction, a verb (e.g., a linking verb), or a combination thereof.

In some embodiments, the parsing of the communication into one or more text strings further includes applying a semantic analysis computational model 222. The semantic analysis computational model 222 provides various natural language processes for evaluating and providing a characteristic of a communication. These tasks include determining a synonym of a word in the communication and/or the one or more text strings, translating a first language into a second language, a question and answer systems, and the like. In some embodiments, a portion of a text string includes a slang word or a word that would provide improved context to a communication of the text string if substituted with a different word. Consider the text string of “To keep my room cool, I bought a cool new air condition machine at the local store last week.” The word “cool” in the above text string is utilized as both a slang word meaning good, and as a conventional definition of the word meaning a low temperature. Accordingly, the wellness system 200 can substitute and/or comprehend the implementation of the slang of the word “cool” to mean good, aiding in an evaluating and providing of a characteristic.

Block 432. Referring to block 432, in some embodiments, the data set associated with the one or more wellness programs 210 includes a weighted average of a subset of attributes in the set of attributes assigned to the first client. For instance, in some embodiments, the weighted average of the subset of attributes is determined using a composite matching score. In some embodiments, this score is a weighted average of the attribute-to-title matching having a first weight (e.g., of about 40%) that is a portion of the attributes 216 that appear in the title of a respective wellness program 210. In some embodiments, this score includes attribute-to-description matching having a second weight (e.g., of about 30%) that is a portion of the attributes that appear in the description of the respective wellness program 210. In some embodiments, this score includes a focus matching having a third weight (e.g., of about 10%) that is a first function of a number of topics presented in the both the request for the wellness program (e.g., block 404 of FIG. 4A) and one or more wellness program attributes 216 and a number of topics in the request and the wellness program 210. In some embodiments, this score includes medical conditions having a fourth weight (e.g., of about 10%) that is a second function of a number of medical condition attributes 216 presented in both request for the wellness program 210 and the topic attributes 216 of a respective wellness program 210 with a of number of medical conditions attributes 216 in the query and the program. Furthermore, in some embodiments, the score includes a user profile 208 matching having a fifth weight (e.g., of about 10%) that is a third function of an average similarity between personal data provided by the client in the user historical performance data set 212 and wellness program historical performance data 218 of one or more clients that have already successfully completed the program the wellness program 210 and provided a positive feedback for the completed wellness program 210. By ensure that only those that provided positive feedback for the completed wellness program 210, the method ensures that the client is matched with the coach that provides the best and most engaging wellness programs for that particular client.

In some embodiments, the data set associated with the one or more wellness programs 210 that is a respective result provided by a respective computational model in the plurality of computational models includes a quality index, such an intelligent quality and reputation index, that is obtained for each wellness program 210. In some embodiments, the quality index includes a score of client satisfaction, a score of scientific validity and/or depth, a score of popularity, or a combination thereof.

Block 434. Referring to block 434, in some embodiments, the data set associated with the one or more wellness program 210 includes a first return of investment (ROI) of the first client and/or a second return on investment of a respective coach associated with a coaching profile in the set of coaching profiles. In some embodiments, ROI includes a function of a respective coach achievement score for a particular attribute 216. In some embodiments, the ROI includes assign every client to a single coach to maximize a total expected ROI when a number of clients per coach is less than or equal to a capacity of a respective coach. As used herein, “capacity” is a number of clients the respective coach is able to lead.

Block 436. Referring to block 436 of FIG. 4E, the method 400 includes collectively considering each respective result that is provided by each respective computational model 222 in the plurality of computational models 222. In some embodiments, this collective consider is performed by the plurality of computational models 222. As such, this collective considering requires a computer (e.g., wellness system 200 of FIGS. 2A and 2B and/or client device 300 of FIG. 3) to be used because such considerations cannot be mentally solved. In other words, given an input to the computational model to collectively consider each respective result, the computational model output needs to be determined using a computer rather than mentally in such embodiments. From this, the method 400 produces producing a set of at least one coaching profile (e.g., user profile 208 associated with the coach) and at least one wellness program 210. In some embodiments, the set of the at least one coaching profile and the at least one wellness program 210 includes a list of wellness programs 210 and coaches with the highest expected quality index for the set of attributes 216 assigned to the client.

Blocks 438. Referring to blocks 438 and 440, in some embodiments, the at least one coaching profile and the at least one wellness program 210 have a one to one relationship in the set. In this way, this ensures that each respective coach associated with a coaching profile in the one or more coaching profile has a corresponding wellness program 210 in the at least one wellness program 210. In this way, should the client view the set of the at least one coaching profile and the at least one wellness program 210 one dimensionally (e.g., viewing the at least one coaching profile without viewing the at least one wellness program 210). However, the present disclosure is not limited thereto.

Block 440. Referring to block 440, in some embodiments, the at least one coaching profile and the at least one wellness program 210 have a one to many relationship in the set. In this way, this ensures that each respective coach associated with a coaching profile in the one or more coaching profile has at least one corresponding wellness program 210 in the at least one wellness program 210, which provides an emphasis on matching a coach with the client.

Furthermore, in some embodiments, the set of at least one coaching profile and the at least one wellness program includes at least 2 coaching profiles and/or wellness programs, at least 5 coaching profiles and/or wellness programs, at least 10 coaching profiles and/or wellness programs, at least 25 coaching profiles and/or wellness programs, at least 50 coaching profiles and/or wellness programs, at least 100 coaching profiles and/or wellness programs, at least 150 coaching profiles and/or wellness programs, at least 250 coaching profiles and/or wellness programs, at least 600 coaching profiles and/or wellness programs, at least 1,000 coaching profiles and/or wellness programs, at least 2,500 coaching profiles and/or wellness programs, at least 5,000 coaching profiles and/or wellness programs, at least 10,000 coaching profiles and/or wellness programs, at least 100,000 coaching profiles and/or wellness programs, or a combination thereof.

Block 442. Referring to block 442, the method 400 includes communicating to a remote device (e.g., client device 300 of FIG. 3) that is associated with the first client the set of the at least one coaching profile and the at least one wellness program. This communicating is performed in electronic form. In this way, the first client is matched with a coach by way of the set of the at least one coaching profile and the at least one wellness program 210.

In some embodiments, the at least one wellness program 210 of the set is displayed on the client device 300 of the client. In some such embodiments, the at least one wellness program 210 sorted by a weighted average between composite matching score and a quality index, such as in descending order.

Block 444. Referring to block 444, in some embodiments, the first client is associated with an enterprise that has vetted the coach. In some embodiments, the coach is vetted by a person that is an employee at the enterprise before inclusion in the set of the at least one or more coaching profiles and the at least one wellness program 210. However, the present disclosure is not limited thereto. For instance, in alternative embodiments, the coach is vetted by a person that is other than an employee at the enterprise before inclusion in the set of the at least one or more coaching profiles and the at least one wellness program 210.

In some embodiments, the set of at least one coach and at least one wellness program 210 is communicated to a subject associated with the enterprise. For instance, in some embodiments, after registering with the wellness system, the enterprise client gets access to a survey for clients that are employees of the enterprise client. In some embodiments, this survey completed by the clients that are employees and received by the wellness system 200. Then, the method 400 provides the enterprise client with a list of attributes 316 related to conditions and concerns found by the plurality of computational models based on the responses provided by the clients that are employees.

In some embodiments, this vetting, including the matching of the client with the coach through the method 400, allows for a gamification of coaching. For instance, the method encourages coaches to spend more time engaging with clients to get qualified for coaching opportunities with enterprise clients based on the user historical performance data set 212 associated with a respective coach, which is reviewable, at least in part, by the enterprise. This feature of vetting creates a beneficial cycle where the more coaches spend time on the platform, and the more they generate content and the wellness programs 210 are deemed complete.

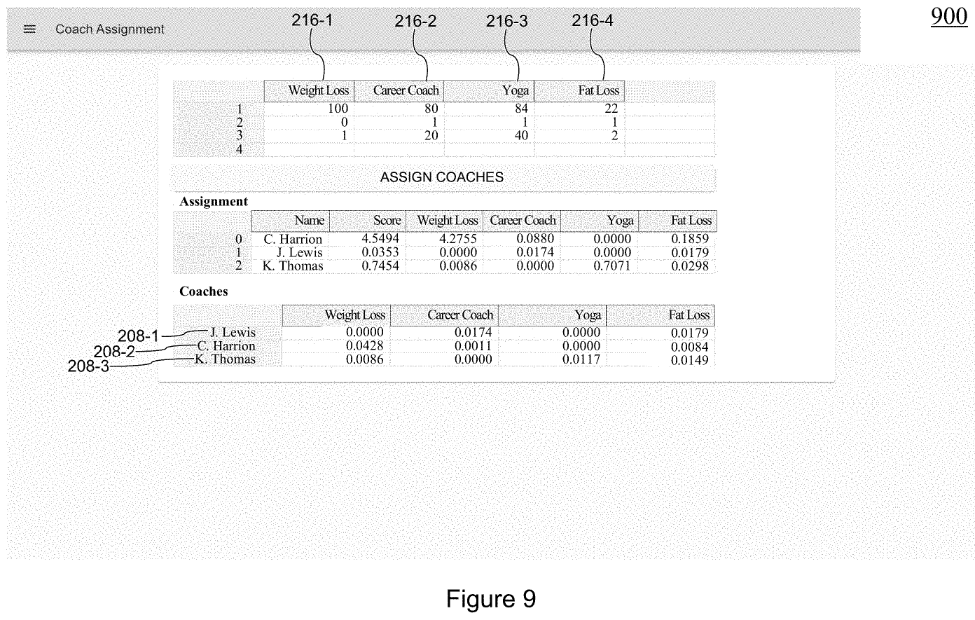

Block 446. Referring to block 446, in some embodiments, the method 400 further includes generating, for display at the client device 300, a listing of the set of the at least one coaching profile and the at least one wellness program. For instance, referring briefly to FIG. 9, a listing of a first set of three coaching profiles is presented for the first client based on the corresponding plurality of responses the first client provided to the survey (e.g., block 404 of FIG. 4A) including a prompt regarding desired weightless from the wellness program 210, career coaching advice from a respective coach of the wellness program 210, an emphasis of yoga attribute 216, and an emphasis on fat lass.

Block 448. Referring to block 448, in some embodiments, the method 400 further includes matching the first coach with the first client, in the corresponding plurality of clients in accordance with an identification, by the plurality of computational models, that the first coach is a respective coach that best matches with a respective attribute of the first client.

In some embodiments, this matching opens a communication channel between the first coach and the first client, such as the communication channel of FIG. 10 or 11.

As such, the matching of the client and the coach requires a computer (e.g., wellness system 200 of FIGS. 2A and 2B and/or client device 300 of FIG. 3) to be used because the matching provided by the plurality of computational models 222 cannot be mentally solved. In other words, given an input to the model, the model output needs to be determined using a computer rather than mentally in such embodiments.

Referring briefly to FIG. 6, a user interface is provided for optimizing a description of a respective wellness program. Here, an example description of “Learn to Play Tennis Like Roger Federer,” is provided to the systems and methods of the present disclosure. To improve the description, the systems and methods of the present disclosure compares the description with one or more descriptions from a respective communications corpus 226 associated with a wellness program 210 or publication. In some embodiments, this comparison is by a neural network computational model that is trained to transform arbitrary text to a fixed-length vector and produce similar vectors for descriptions of the respective wellness program from the same attributes the coach provided and far vectors for different attributes. In some embodiments, a point cloud labeled by attribute 216 is obtained by passing all existing descriptions of wellness programs 216, or a subset thereof, through this computational model. In some embodiments, for an obtained embedding vector, one or more nearest neighbors are chosen from the point cloud of labeled descriptions. Then for every attribute 216 from the chosen vectors a probability of attribute 216 presence in the description is obtained as a portion of vectors labeled with this attribute 216 among chosen nearest neighbors. These attributes 216 and probabilities are shown to the coach, as illustrated by FIG. 11. In some embodiments, if the most probable attribute 216 is below a threshold value (e.g., 50%), a hint is displayed to the coach to change the description to more attribute 216 specific recommendations.

### Example 1: A First Wellness System 200

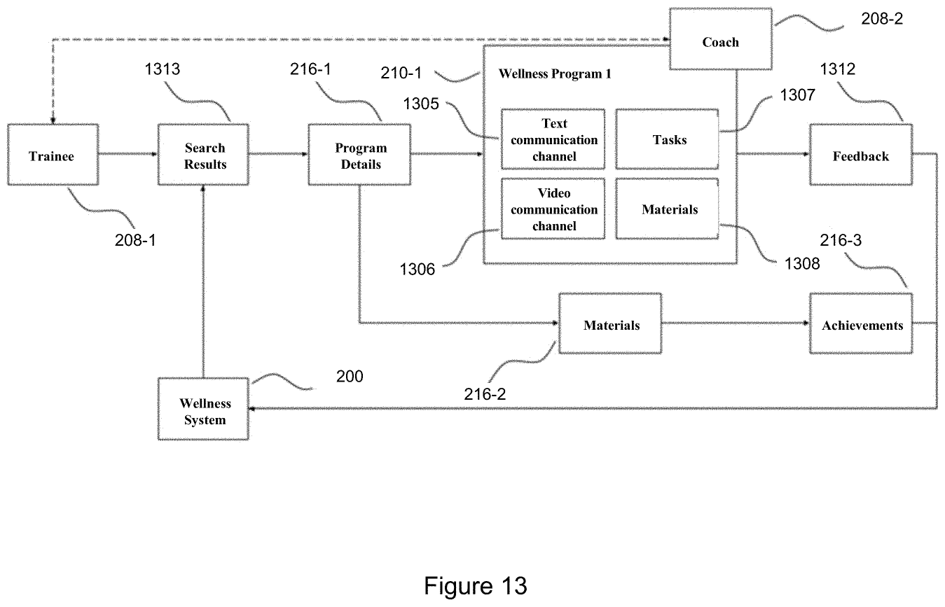

Referring briefly to FIG. 5 through FIG. 11 and FIG. 13, in some embodiments, a respective client 208-1 within a wellness system 200 is provided access a client application 320 that enables the respective client 208-1 to: search for a respective wellness program 210, join the respective wellness program 210, complete the respective wellness program 210 (e.g., accomplish one or more goals associated with the respective wellness program 210), provide feedback for the respective wellness program 210, provide feedback for a coach 208-2 associated with the respective wellness program 210, or a combination thereof. At step 1313, in some embodiments, the respective client 208-1 provides a search to find the respective wellness program 210 matching the needs of the respective client 208-1, such as specified by a set of attributes assigned to the respective client 208-1. In some embodiments, a subject other than the respective client 208-1 In some embodiments, the respective client 208-1 views an attribute 216 associated with a wellness program 210, including a title of the respective wellness program 210, a description of the respective wellness program 210, a price of the respective wellness program 210, a length of the respective wellness program 210, information about a coach 208 associated with the respective wellness program 210, or a combination thereof. In some embodiments, to start the respective wellness program 210, the respective client 208-1 pays (e.g., provides payment information to the wellness system 200 and/or the respective coach 208-2), such as by using an input of the client device 300 associated with the respective client 208-1. In some embodiments, after starting the respective wellness program 210, the respective client 208-1 gets access to materials 1308 of the respective wellness program 210 such as books provided by the respective coach 208-2, tasks 1307, and communication channels 1305, 1306 associated with the respective wellness program 210 (e.g., one or more corpus of communications 226 associated with the respective wellness program 210). In some embodiments, the respective client 208-1 specifies one or more goals in the set of attributes 216 to achieve after completion of the respective wellness program 210 (e.g., weight loss 216-1 of FIG. 9, fat loss 216-4 of FIG. 9, etc.). In some such embodiments, each goal is an arbitrary attribute 216 describing an intention and/or a wish of the respective client. In some embodiments, each goal includes a weight showing relative interest of the respective client 208-1 for this attribute (e.g., a high weight of “22” for first request for a coach of FIG. 9 and a low weight of “2” for third request for the coach of FIG. 9). In some embodiments, after the respective wellness program 210 is deemed complete for the respective client 208-1, the respective client 208-1 specifies a percentage of achievement for each goal assigned to the respective client 208-1 at the start of the respective wellness program 210. In some embodiments, the respective client 208-1 provides feedback on the respective wellness program 210 when deemed complete by filling out a first survey, such as filling the following rating fields: one or more results of each goal attribute 216, a depth of the respective wellness program 210 (e.g., rating a difficulty level of the respective wellness program 210, an amount of information provided by the respective wellness program 210, etc.), a rating of materials provided through the respective wellness program 210, manners and ease of communication of the respective coach 208-2 conducting the respective wellness program 210, recommendation to other clients and/or coaches for future enrollment in the respective wellness program 210, or a combination thereof. In some embodiments, the respective client 208-1 provides feedback for the respective coach 208-2 associated with the respective wellness program 210 by filling out a second survey, such as filling the following fields: one or more results of each goal attribute 216, a competence of the respective coach 208-2, attentiveness of the respective coach 208-2, manners and ease of communication of the respective coach 208-2, recommendation to others for the respective coach 208-2, or a combination thereof. In some embodiments, the respective wellness program 210 is deemed completed by the respective client 208-1 when a threshold number of tasks is satisfied (e.g., 50% of tasks are completed, 60% of tasks are completed, 75% of tasks are completed, 80% of tasks are completed, 95% of tasks are completed, etc.) by the respective client 208-1 and/or the respective client 208-1 has provided feedback. In some embodiments, the feedback provided by the respective client 208-1 must satisfy a threshold feedback score such as an average score at least 0.8 (e.g., B−), at least 0.85 (e.g., B+), at least 0.9 (e.g., A−), etc. However, the present disclosure is not limited thereto. In some embodiments, after the respective client 208-1 is deemed to have finished the respective wellness program 210 (e.g., completes or fails a threshold number of tasks associated with the respective wellness program 210), a plurality of computational models 222 is utilized to provide a set of results. In some embodiments, the set of results includes at least two result that are evaluated by the wellness system 200 for this client 208-1/wellness program 210 pair, such as a first engagement result and a second quality result. In some embodiments, each result is a quantity, and, therefore, in the form of a data set. However, the present disclosure is not limited thereto. In some embodiments, the first engagement result is determined based on a portion of completed tasks, a portion of days by the respective client and/or the respective coach with activity in a communication channel associated with the respective wellness program 210, a presence of one or more task overdues, or a combination thereof. In some embodiments, each result provided by a respective computational model 222 is a quantity associated with an attribute 216 (e.g., first quality attribute 216-1, second engagement attribute 216-2, etc.) that lies between 0 and 1 and is expressed as float numbers or in percent from 0 to 100. In some embodiments, the second quality result is a portion of goal achievements weighted by goal importance set by the respective client 208-1 when starting the respective wellness program 210. In some embodiments, the quality result is provided for each wellness program 210 and each coach 208 associated with the wellness system 210, such as by determining an average quality across all clients associated with the respective wellness program 210 for a corresponding wellness program 210 and/or a corresponding coach 208 with engagement as a weight. However, the present disclosure is not limited thereto. For instance, in some embodiments, the plurality of computational models 222 is utilized to determine a popularity result for a respective coach 208-2 and/or a respective wellness program 210. In some embodiments, the popularity result is determined based on a number of views of the respective wellness program 210 (e.g., views of the description and/or focus attributes 216 of the respective wellness program 210), a number of followers of the respective wellness program 210, a number of purchases of the respective wellness program 210, or a combination thereof. In some embodiments, the popularity result is a number equal or greater than 0. In some embodiments, the respective wellness program 210 is associated with a unique popularity number, such that no two wellness programs 210 share the same popularity number result. As such, the wellness system 200 requires a computer (e.g., wellness system 200 of FIGS. 2A and 2B and/or client device 300 of FIG. 3) to be used because the results provided by the plurality of computational models 222 cannot be mentally solved. In other words, given an input to the model, the model output needs to be determined using a computer rather than mentally in such embodiments. However, the present disclosure is not limited thereto.

### Example 2: Matching a Respective Client 208 and a Wellness Program 210

Referring to FIGS. 9, 12, and 14, in some embodiments, a request for a wellness program 210 is received from a client device 300 associated with a respective client 208-1. The request includes a search query of a set of attributes 216 assigned to the respective client 208-1. In some embodiments, the set of attributes 216 assigned to the respective client 208-1 includes one or more keywords associated with the respective wellness program 210, a focus or discipline of the respective wellness program 210, one or more medical conditions associated with the respective wellness program, personal information from a user profile 208 associated with the respective client 208, or a combination thereof. In some embodiments, keywords include arbitrary words or phrases to find in a title and description of the respective wellness program 210. In some embodiments, the focus is a list of attributes 216 the respective client 208-1 is interested in with a corresponding weight assigned to every attribute 2016. In some embodiments, each topic is entered through a list where the respective client 208-1 is able to add topics, assign a weight or remove the weight, and the like. In some embodiments, the respective client 208-1 chooses one or more topics from a predefined list of topics. In some embodiments, the client selects one or more medical conditions through another editable list with ability to choose from a predefined list. In some embodiments, personal data is provided by the respective client 208-1 during/after registration and stored in a corresponding user profile 208 associated with the respective client.

In some embodiments, the respective programs is matched to the set of attributes 216 assigned to the respective client 208-1, and, therefore, included in a set if at least one coaching profile and at least one wellness program provided to the respective client 208-1 in response to the request, such as by using a plurality of computational models 222 to produce a result including a composite matching score. In some embodiments, this score is a weighted average. In some embodiments, the weighted average includes: Keywords-to-Title Matching (40%) that is a portion of the keywords that appear in the title of the respective wellness program 210; Keywords-to-Description Matching (30%) that is a portion of the keywords that appear in the description of the respective wellness program 210; Focus Matching (10%) that is a twice a number of topics presented in the both the set of attributes 216 assigned to the client 208-1 and the one or more attributes 216 associated with the respective wellness program 210 divided by the sum of number of topics in the request and the respective wellness program 210; Medical Conditions (10%) that is a twice a number of conditions presented in the both the respective client 208-1 request and the topic attributes 216 of the respective wellness program 210 divided by the sum of number of conditions in the query and the program; and Personal Data Matching (10%) that is an average similarity between personal data of the user profile 208 provided by the respective client 208-1 and personal data of different clients that have already successfully completed the respective wellness program and provided a positive feedback (e.g., satisfied the threshold feedback score). However, the present disclosure is not limited thereto.

In some embodiments, the set of the at least one coaching profile and the at least one wellness program 210 is presented on the display of the client device 300 to the respective client 208-1. In some embodiments, the at least one wellness program 210 is sorted, such as by a weighted average between composite matching score defined above (75%) and quality (25%) in descending order. However, the present disclosure is not limited thereto. In some embodiments, the respective client 208-1 is able to navigate to a corresponding page for the respective wellness program 210 from the set of the at least one coaching profile and the at least one wellness program 210. As such, the matching of the client and the coach requires a computer (e.g., wellness system 200 of FIGS. 2A and 2B and/or client device 300 of FIG. 3) to be used because the matching provided by the plurality of computational models 222 cannot be mentally solved. In other words, given an input to the model, the model output needs to be determined using a computer rather than mentally in such embodiments.

### Example 3: Recommending Improvements for a Wellness Program 210

Referring to FIGS. 6, 8, and 15, in some embodiments, when a title of the respective wellness program 210 is specified by the coach, the wellness system 200 utilizes the plurality of computational models 222 to provide one or more recommendations on how to improve the wellness program. In some embodiments, the one or more recommendations seek to make the description of the respective wellness program 210 clear (e.g., clean of spelling and/or grammar errors) and/or concise. In some embodiments, the one or more recommendations seek to adjust a structure or tweak the respective wellness program 210 to optimize for the goal of the respective wellness program 210. However, the present disclosure is not limited thereto.

In some embodiments, the one or more recommendations to improve the description of the respective wellness program 210, the wellness system 200 uses the plurality of computational models compares a first description provided by the respective coach 208-2 with one or more descriptions from a corpus of communications 226 associated with the respective wellness program 210, such as a respective corpus of communications 226 obtained from existing open resources and/or a different wellness program 210. In some embodiments, the respective corpus of communications is configured or defined by a respective computational model 222 (e.g., a neural network computational model 222). In some such embodiments, the respective computational model 222 is trained on a training data set in order to transform arbitrary text to a fixed-length vector. In some embodiments, the respective computational model 222 is trained to produce similar vectors for attributes associated with a respective wellness program 210 associated with a first attribute 216-1 and far vectors for different attributes 216. However, the present disclosure is not limited thereto. In some embodiments, a point cloud of the respective computational model 222 that is labeled by topic is obtained by passing one or more existing program descriptions associated with the wellness programs of the wellness system by the respective computational model 222. Accordingly, in some such embodiments, when the respective coach 208-2 adds or changes the description of the respective wellness program 210, the changed description is passed through the respective computational model 222. As such, in some embodiments, for the obtained embedding vector, one or more nearest neighbors is chosen from the point cloud 25 of labeled descriptions. Accordingly, in some embodiments, for every topic (e.g., attribute 216) from the chosen vectors a probability of topic presence in the description is obtained as a portion of vectors labeled with this topic among chosen nearest neighbors. However, the present disclosure is not limited thereto. In some embodiments, the topics and probabilities form a portion (e.g., some or all) of a revised set of attributes 216 that is shown to the coach to improve the wellness program 210. In some embodiments, if the plurality of computational models 222 determine that the most probable topic is below a threshold score (e.g., about 40%, about 50%, about 55%, about 60%, etc.), a hint to change description to more topic-specific descriptions is presented to the respective coach 208-2. From this, the one or more recommendations improve the respective wellness program 210 by ensuring that the description is configured to draw clients to participate in the respective wellness program 210. However, the present disclosure is not limited thereto. For instance, in some embodiments, a recommendation in the form of a maximization of one or more attributes 216 is provided to ensure that each attribute 216 in the set of attributes 216 is a best fit. In some embodiments, the one or more attributes 216 evaluated by the plurality of computational models 222 include: a title of the respective wellness program 210, a description of the respective wellness program 210, a focus of the respective wellness program 210, duration of the respective wellness program 210, price of the respective wellness program 210, number of materials of the respective wellness program 210, or a combination thereof. In some embodiments, optimal values for these attributes 216 are found as a weighted average of attributes 216 of each respective wellness program 216 in the given topic of the respective wellness program 210. In some embodiments, the optimal values for these attributes 216 are further found by related topics with the respective wellness program 210 based on topic similarity as weights. In some embodiments, these values are shown to the respective coach when creating the respective wellness program 210.

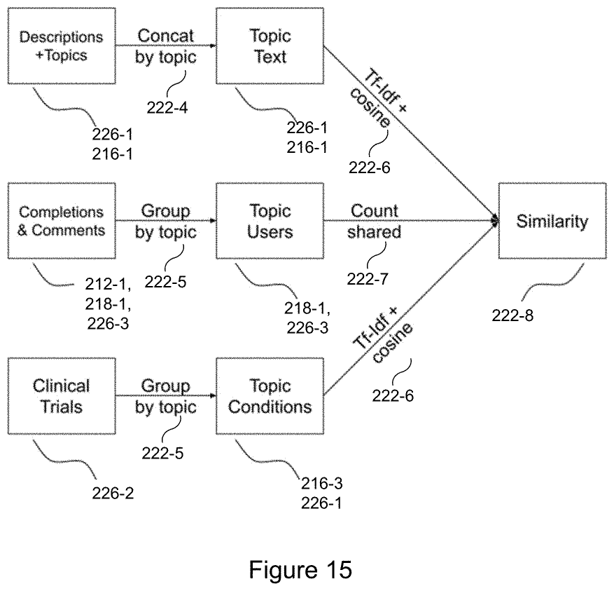

Referring briefly to FIG. 15, in some embodiments, when the respective coach 208-2 creates a new wellness program (e.g., using a client device 300), the wellness system 200 provides the respective coach 208-2 with a set of revised attributes 216 (e.g., presents a listing of the set of related attributes), such as one or more related attributes 216 (e.g., related topic attributes, related focus attributes, etc.), to one specified for the respective wellness program 210 being created by the respective coach 208. In some embodiments, the set of related attributes 216 is produced by using the plurality of computational models 222 including a compound attribute similarity measure. In some embodiments, this similarity is based on a plurality of data sets resultant from one or more computational models 222 including a first evaluation, a second evaluation, a third evaluation, or a combination thereof.

In some embodiments, the first result includes a similarity between existing descriptions for one or more wellness programs 210 hosted by the wellness system and the description provided by the respective coach 208-3. In some embodiments, these existing descriptions are taken from historical data sets 212, 214 of the wellness systems and/or open resources (e.g., corpus of communications 226 based on scientific publications). In some embodiments, for every topic descriptions of a plurality of wellness program 210 that includes some or all of the wellness programs 210 hosted by the wellness system 200 from this topic are concatenated. Thus, in some such embodiments, a single corpus of communications 226 for every topic is obtained. In some embodiments, these documents are transformed to fixed-length vectors using a term frequency-inverse document frequency (TF-IDF) transform. Additional details and information regarding TF-IDF can be found at Qaiser et al., 2018, “Text Mining: Use of TF-IDF to Examine the Relevance of Words to Documents,” International Journal of Computer Applications, 181(1), pg. 25; Aytu\check{g} et al., 2021, “Weighted Word Embeddings and Clustering-based Identification of Question Topics in MOOC Discussion Forum Posts,” Computer Applications in Engineering Educations, 29(4), pg. 675, each of which is hereby incorporated by reference in its entirety. In some embodiments, the first result is based on a cosine similarity applied to every possible pair of these aforementioned vectors, whereby pairwise similarity between topics is produced.

In some embodiments, the second result is based on one or more attributes 216 of one or more wellness programs 210 other than the respective wellness program 210 by the same user (e.g., the same respective coach, the same respective client, etc.). For instance, in some embodiments, for every pair of topics a number of clients deemed to have passed a wellness program 210 from both of these topics is obtained from a respective historical data set 212, 214 of the wellness system 100. In some embodiments, this value result is considered with the number of clients who have been deemed to have passed one or more wellness programs 210 from these topics. Then, in some embodiments, for each wellness program 210 and/or corpus of communications 226 the wellness system 200 obtains from one or more open resources, a number of users posted comments for the wellness programs 210 from every pair of topics is obtained and then divided on a product of number of comments for all wellness programs 210 from these topics. In some embodiments, the obtained values are combined in a ratio 2:1 producing a demand-based topic similarity.

In some embodiments, the third result includes a topic similarity measure that is based on open data about clinical trials (e.g., second corpus of communications 226-2 including one or more clinical trial data sets, etc.). In some embodiments, this second corpus of communications 226-2 includes a list of one or more clinical trials that is further annotated with one or more conditions (e.g., diagnoses) of the participants of the one or more clinical trials and one or more interventions (e.g., type of treatment) applied to a respective participant. In some embodiments, all condition attributes 216 are labeled by one or more topic attributes 216 by checking for the presence of the topic name or a variant of the topic name in a condition. In some embodiments, the one or more topic variants include an initial topic name, a name with spaces replaced with hyphens, slashes replaced with spaces and so on. For instance, consider a first topic attribute name of “Dachshund” and a first variant of “Weiner Dog,” a second topic attribute name of “long distance running” and a second variant of “ultramarathon,” and the like. In some embodiments, for every topic attribute, a count of times a condition attribute appears in one or more clinical trials with a corresponding intervention labeled with this topic is obtained. Thus, in some such embodiments, the second corpus of communications 226-2 includes a document-term matrix that appears with topics as documents and conditions as words. Then, in some embodiments, using the plurality of computational models 222, such as Tf-Idf followed by a cosine similarity, is applied to get a pairwise topic similarity.

In some embodiments, the first result, the second result, the third result, or the combination thereof is combined (e.g., eight computational model 222-8 of FIG. 15) based on a requirement to satisfy a threshold of condition-based similarity (e.g., 50%, 60%, 65%, etc.), a threshold comment-based similarity (e.g., 20%, 25%, etc.), a threshold text-based compound topic similarity measure (e.g., 15%, 20%), or a combination thereof. However, the present disclosure is not limited thereto.

As such, the one or more results and/or one or more recommendations provided by the wellness system 200 requires a computer (e.g., wellness system 200 of FIGS. 2A and 2B and/or client device 300 of FIG. 3) to be used because the one or more results and/or one or more recommendations provided by the plurality of computational models 222 cannot be mentally solved. In other words, given an input to the model, the model output needs to be determined using a computer rather than mentally in such embodiments.

### Example 4: Obtaining and/or Evaluating a Corpus of Communications 226

In some embodiments, the wellness system 200 obtain one or more data sets (e.g., a corpus of communications 226 from an external source.

In some embodiments, a first external source for a portion of one or more corpus of communications 226 is a public forum or conversation related to a respective attribute 215, such as related to cardiovascular health. In some embodiments, the first external source is used as a training set of data for one or more computational models 222. In some embodiments, the one or more computational models 222 use the corpus of communications 222 associated with the first external source to determine a probability a given conversation of a respective coach 208-2 belongs to a given topic, such as to determine if the respective coach 208-2 is discussing the correct topic for the respective wellness program 210.

In some embodiments, a second external source for the portion of the one or more corpus of communications 226 is scientific (e.g., academic) publications. In some embodiments, the scientific publication is annotated with conditions and interventions described therein, which form a basis for a respective computational model 222. Accordingly, these scientific publications are used by the plurality of computational models 222 to produce one or more connections between respective attributes 216, such as between topics and conversations.

In some embodiments, a third external source for the portion of the one or more corpus of communications 226 is the descriptions and/or topics provided by the respective coach 208-2 when creating the respective wellness program 210. In some embodiments, these descriptions are then used by the plurality of computational models 222 to classify one or more attributes 216 for a revised set of attributes 216, such as one or more topic coverage recommendations and the like.

In some embodiments, the first external source, the second external source, the third external source, or a combination thereof is merged with internal data sets (e.g., user profiles 208 and/or historical data sets 212, 214) of the wellness system, such as to obtain one or more intermediate results by the plurality of computational models 222. In some embodiments, the one or more intermediate results is used by the wellness system 200 handle incoming queries provided by the respective client 208-1 and/or produce results returned to the respective client 208-1.

As such, the obtaining and/or the evaluating of the corpus of communications 226 provided by the wellness system 200 requires a computer (e.g., wellness system 200 of FIGS. 2A and 2B and/or client device 300 of FIG. 3) to be used because the obtaining and/or the evaluating provided by the plurality of computational models 222 cannot be mentally solved. In other words, given an input to the model, the model output needs to be determined using a computer rather than mentally in such embodiments.

### Example 5: Matching a Respective Coach and a Respective Client Vetted by Entity of the Respective Client

In some embodiments, the systems and methods of the present disclosure provide facilitating matching the respective coach and the respective client when the respective client is an employee of the entity. In some embodiments, the entity vets the respective coach and/or a plurality of coaches including the respective coach. In some embodiments, the entity registers with the wellness system 200 (e.g., onboarding, creates a user profile 208 associated with the entity, etc.). In some embodiments, the entity gets access to a survey that is provided to a plurality of clients, in which each client in the plurality of clients is an employee of the entity. In some embodiments, the entity gets access to a predetermined survey provided by the wellness system 200, provided by one or more coaches 208, or a combination thereof. In some embodiments, the entity creates the survey using a client application 320. The survey is configured to receive a set of attributes from each client. For instance, in some embodiments, this survey is to be downloaded at a respective client device fill by a respective client using the client device 300. After receiving the set of attributes 216 (e.g., block 404 of FIG. 4A) from a survey, the set of attributes 216 is considered by a plurality of computational models 222. In some embodiments, this considering forms a collective set of attributes, such as a list of medical conditions and health-related concerns of clients collectively. In some embodiments, for every coach and/or wellness program 210, the wellness system 200 obtains a quality result and confidence result from the plurality of computational models 22. In some embodiments, the quality result (e.g., a fixed attributed based on the quality result produced by the wellness system 200) is an average achievement score weighted by user engagement. In some embodiments, the confidence result is a logarithmic function of the number of sessions for a particular coach and/or wellness program 210. However, the present disclosure is not limited thereto. In some embodiments, an expected quality result is provided by the plurality of computational models 222, such as based on a product of the quality result and the confidence result. In some embodiments, the wellness system 200 provides the respective client 208-1 with a data set that includes a list of topic attributes 216 related to one or more conditions and concerns found. In some embodiments, the data set includes at least one coaching profile and at least one wellness program 210 that have the highest expected quality results in these topic attributes 216.

As such, the matching of the client and the coach requires a computer (e.g., wellness system 200 of FIGS. 2A and 2B and/or client device 300 of FIG. 3) to be used because the matching provided by the plurality of computational models 222 cannot be mentally solved. In other words, given an input to the model, the model output needs to be determined using a computer rather than mentally in such embodiments.

### Example 6: Return-On-Investment for a Plurality of Clients by a Plurality of Computational Models 222

In some embodiments, when matching a respective client 208-1 with a coach and/or a wellness program 210, the wellness system 200 provides the respective client 208-1 with a return-on-investment (ROI) estimation using the plurality of computational models 222. In some embodiments, the ROI is determined based on a respective quality result for each coach for every condition attributed 216 based on goal achievement score of the respective coach. In some embodiments, the estimated ROI is provided for each attribute or a subset of attributes (e.g., condition attributes 216) in the set of attributes 216 assigned to the respective client 208-1 and/or obtained from open resources (e.g., scientific publications). In some embodiments, the estimated ROI for every coach and attribute 216 pair is provided, such as based on a product of the achievement score result of the respective coach and the expected ROI for a corresponding attribute 216. In some embodiments, each client is assigned to a respective coach in a plurality of coaches to maximize total expected ROI while the number of clients per coach is less or equal to a capacity of the respective coach. In some such embodiments, the capacity of the respective coach includes a number of clients the respective coach is able to lead. In some embodiments, the number of clients the respective coach is able to lead is determined by the plurality of computational models 222. In alternative embodiments, the number of clients the respective coach is able to lead is determined by the respective coach. Furthermore, in some embodiments, a total expected ROI is a sum of the expected ROI for each client in the plurality of clients.

In some embodiments, the systems and methods of the present disclosure use the expected ROI and the total ROI to get several resultant data sets including a number of employees that satisfy a threshold score, such as greater than or equal to 80% expected achievement score, condition coverage, and the like.

In some embodiments, the systems and methods of the present disclosure associated the respective coach with a corresponding identifier that is configured to corresponding to a vetted status. In some embodiments, the corresponding identifier is associated the respective coach when the user profile of the respective coach satisfies a threshold condition using the plurality of computational models 222. In this way, in some such embodiments, an element of gamification is utilized by the system and methods of the present disclosure to incentivize the respective coach to spend more time engaging with the wellness system 200. For instance, in some embodiments, for every level of achievement in a plurality of levels of achievement, whether it is a minimum number of hours engaging with the wellness system 200, minimum number of clients deemed to have completed a corresponding wellness program 210 must be satisfied by an ascending (e.g., escalating) threshold minimum level of achievement and minimum level of review. Accordingly, in some such embodiments, the higher the activity of the respective coach, the more probability that the systems and methods of the present disclosure can obtain enough data sets and information to for a plurality of computational models to determine resultant data sets required to match a client with a best coach, such as confidence and quality scores of a respective coach. In some embodiments, once a certain threshold level is achieved by the respective coach, the respective coach reaches an administrator status of the wellness system. However, the present disclosure is not limited thereto.

As such, the matching of the client and the coach requires a computer (e.g., wellness system 200 of FIGS. 2A and 2B and/or client device 300 of FIG. 3) to be used because the matching provided by the plurality of computational models 222 cannot be mentally solved. In other words, given an input to the model, the model output needs to be determined using a computer rather than mentally in such embodiments.

### Example 7: Oversight of a Communication Channel of a Wellness Program 210

In some embodiments, the systems and methods of the present disclosure provide oversight of one or more communication channels associated with a respective wellness program, such as first communication channel of FIG. 11 or second communication channel of FIG. 12. In some embodiments, the information exchanged through the communication channel is evaluation by the plurality of computational models 222, such as evaluating for a presence of a medical attribute 215 and other topic-related terms to prove that conversation between the respective coach 208-2 and the respective client 208-1 is not off-topic. In some embodiments, the related terms are obtained from a topic attribute classifier based on a number of words with the highest Tf-Idf coefficient. In some embodiments, the related terms are obtained from clinical-related data sources including medical conditions, interventions, and other terms extracted from scientific data (e.g., second corpus of communications 226-2 of FIG. 15). However, the present disclosure is not limited thereto. In some embodiments, the systems and methods of the present disclosure determines that a conversation in the communication channel is treated as topic-related when a threshold condition is satisfied. In some embodiments, the threshold condition includes at least one term per day for 90% of days this communication exists. However, the present disclosure is not limited thereto. For instance, in some embodiments, if the threshold condition is satisfied, one or more notifications are sent to a respective client device 300 associated with the respective coach of the wellness program associated with the communication channel, an entity associated with the respective client of the wellness program, an administrator of the wellness system 200, or a combination thereof. In some embodiments, the systems and methods of the present disclosure utilize the plurality of computational models 222 to analysis a sentiment of a communication channel and/or a user engaging with the communication channel. In some embodiments, the sentiment is utilized by the plurality of computational models 222 to further determine if abuse and similar improper actions are occurring.

As such, the oversight of the communication channel requires a computer (e.g., wellness system 200 of FIGS. 2A and 2B and/or client device 300 of FIG. 3) to be used because the oversight provided by the plurality of computational models 222 cannot be mentally solved. In other words, given an input to the model, the model output needs to be determined using a computer rather than mentally in such embodiments.

### Example 8: Using Historical and Empirical Data Sets to Match a First Client and a Coach

In some embodiments, the systems and methods of the present disclosure provide obtain and process a plurality of data sets including one or more historical data sets, one or more empirical data sets (e.g., corpus of communications), and a set of attributes assigned to a first client in order to facilitate the match of the first client and the coach.

More particularly, a computer wellness system (e.g., wellness system 200 of FIGS. 2A and 2B) is utilized to host a plurality of wellness programs (e.g., first wellness program 210-1, second wellness program 210-2, . . . , wellness program R 210-R of FIGS. 2A and 2B). Furthermore, each respective wellness program 210 in the plurality of wellness programs 210 is associated with a corresponding coach in a plurality of coaches. Each respective coach in the plurality of coaches is associated with a corresponding user profile in a plurality of user profiles stored by the wellness system 200.

In some embodiments, the systems and methods of the present disclosure receive a request in electronic form for a wellness program (e.g., a request communicated by way of communication network 106 to be match with the wellness program or a respective coach associated with the wellness program).

The request for the wellness program includes a set of attributes from a plurality of attributes (e.g., attributes 216 of FIGS. 2A and 2B). The set of attributes is assigned to the first client. In some embodiments, the set of attributes assigned to the first client is further stored in a corresponding user profile associated with the first client, which allows the wellness system 200 to at least match a second client with a respective wellness program based, at least in part, on a similarity between the set of attributes assigned to the first client and one or more attributes assigned to the second client. However, the present disclosure is not limited thereto.

In some embodiments, the first client is responsible for assigning the set of attributes. For instance, in some embodiments, the first client is presented with one or more prompts (e.g., survey prompts) that is configured to elicit a response (e.g., answer) from the first client, in which the response is associated with one or more attributes 215. A non-limiting example includes the first client choosing one or more attributes from a subset of attributes in the plurality of attributes. In some embodiments, a subject other than the first client is responsible for assigning the set of attributes to the first client. In some embodiments, the subject other than the first client is a first coach that is associated with the respective wellness program, an administrator of the wellness system, or the like. For instance, in some embodiments, the first client has a conversation with the administrator of the wellness system (e.g., through a communication channel, such as a through a short message service, a social media platform, an audio recording, etc.), through which the administrator assigns the set of attributes to the first client based on the conversation with the first client. In this way, the subject acts as a concierge for the first client to match with a respective coach. However, the present disclosure is not limited thereto. For instance, in some embodiments, a plurality of computational models (e.g., computational models 222 of FIG. 2B) provide an evaluation of one or more responses provided by the first client, whereby the evaluation identifies one or more attributes for inclusion in the set of attributes assigned to the first client. For instance, consider a first question asking for a weight of the first client, a first response to the first question that includes a weight of the first client of about 350 pounds. Accordingly, in some embodiments, the plurality of computational models evaluates the first response to identify a first attribute associated with obesity for inclusion in the first set of attributes.

In some embodiments, responsive to the request for the wellness program (e.g., in response to receiving the set of attributes assigned to the first client), the systems and methods of the present disclosure obtain a plurality of coach profiles. The plurality of coaching profiles is each user profile in the plurality of user profiles of the wellness system that is associated with a coach of the wellness system 200. In some embodiments, the plurality of coaching profiles is a subset of each user profile in the plurality of user profiles that is associated with the coach. In this way, each respective coaching profile is associated (or includes) a corresponding one or more wellness programs 210 that is administrated, at least in part, by the corresponding coach. For instance, in some embodiments, the corresponding coach administrates the wellness program 210 by conducting a meeting with a respective client of the wellness program. In some embodiments, the corresponding coach administrates the wellness program 210 by mentoring one or more apprentice coaches (e.g., clients).

Furthermore, each coaching profile includes a corresponding first historical data set. This corresponding first historical data set is configured to at least store information obtained by the systems and methods of the present disclosure, such as usage information, feedback information, the like. In some embodiments, the corresponding first historical data set includes one or more results produced by a respective computational model in the plurality of computational models, such as in accordance with a determination that the one or more results is associated with the corresponding coach. As a non-limiting example, in some embodiments, the corresponding first historical data set includes a quality of the corresponding coach, a quality of a mentor coach of the corresponding coach, a quality of one or more wellness programs associated with the corresponding coach, an accuracy and/or precision of a respective wellness program in the one or more wellness programs associated with the corresponding coach, a relevance quantity of the respective wellness program, a popularity of the corresponding coach, an achievement level of the corresponding coach, a projected return of investment associated with the corresponding, or a combination thereof.

For instance, in some embodiments, the accuracy and/or precision of the respective wellness program is processed by the plurality of computational models based on an average of independently weighted parameters, such as a first parameter of a first weight (e.g., 20%) that is a text-based cosine similarity of Tf-Idf vectors of texts formed by concatenation of a respective description of the respective wellness program 210 assigned by the corresponding coach, a second parameter of a second weight (e.g., 20%) that is a comment-based similarity of a number of users posted comments associated with a respective attribute (e.g., topic), and a third parameter of a third weight (e.g., 60%) that is medical conditions-based cosine similarity between Tf-Idf vectors formed by one or more medical conditions appearing in one or more corpus of communications (e.g., clinical trial data sets) related to one or more attributes 216. However, the present disclosure is not limited thereto.

In some embodiments, the quality of the corresponding coach is determined based on a set of parameters. In some embodiments, the quality of the corresponding coach is based on a first parameter of a first weight (e.g., 70%) and a second parameter of a second weight (30%). For instance, in some embodiments, the first parameter includes an average achievement score for all sessions that the corresponding has administrated for a respective wellness program 210. In some embodiments, the second parameter is an average feedback score across all feedback scores provided by each client associated with the corresponding coach.

Accordingly, each respective coach is enabled to engage with the wellness system 200, either by directly engaging with a respective client (e.g., through a communication channel) or conducting a respective wellness program. In some embodiments, when a respective coach and/or the respective client is conducting the respective wellness program 210, the wellness system 200 obtains one or more historical data sets including a number of completed tasks (e.g., by the respective client and/or the respective coach), a period of time with activity in a respective communication channel (e.g., a percentage of days with chat activity), a number of tasks identified as overdue, or a combination thereof. From this engagement, the wellness system 200 obtains and curates rich historical and empirical data sets. Moreover, coaches are challenged to improve coaching skills, improve wellness program content, service more clients, create new wellness programs, and achieve higher scores within the wellness system 200 in order to increase the engagement by the corresponding coach, thereby increasing the matching of the corresponding coach with clients.

Furthermore, in some embodiments, responsive to the request for the wellness program, the systems and methods of the present disclosure obtain a plurality of wellness programs 210. In some embodiments, a respective wellness program in the plurality of wellness programs is created and/or administrated by a corresponding coach. In this way, the systems and methods of the present disclosure allow the corresponding coach to create, edit, administrate, delete, evaluate, or a combination thereof the respective wellness program. For instance, in some embodiments, the corresponding coach and/or the plurality of computational models is able to assign one or more attributes to the respective wellness program. For instance, in some embodiments, the corresponding coach configures (e.g., through a client device associated with the corresponding coach) a title of the respective wellness program 210 that is identify the respective wellness program 210, a description that provides a summary of contents of the respective wellness program, a focus of the respective wellness program that includes a list of one or more topics or disciplines associated with the respective wellness program (e.g., a first topic of tennis, a second topic of clay court, a third topic of amateur, etc.). In some embodiments, each topic or discipline of the focus includes a weight independently assigned to each topic. Accordingly, the corresponding coach or the plurality of computational models is able to combine optimization targets by independently assigning weights to each topic.

In some embodiments, similar to the first corresponding data set associated with the corresponding first historical performance of the corresponding each, each respective wellness program 210 is associated with a second corresponding data set that is associated with a second historical performance of the respective wellness program. In this way, the second corresponding data set is configured to reflect collective performance of clients and, optionally, one or more coaches, associated with the respective wellness program 210. For instance, in some embodiments, the second corresponding data set includes a quality of the respective wellness program 210, an achievement score of each client associated with the respective wellness program 210, an accuracy and/or precision of the respective wellness program 210, and the like.

For instance, in some embodiments, the quality of the respective wellness system is determined based on a set of parameters. In some embodiments, the quality of the corresponding coach is based on a first parameter of a first weight (e.g., 70%) and a second parameter of a second weight (30%). For instance, in some embodiments, the first parameter includes an average achievement score for all sessions of the respective wellness program 210. In some embodiments, the second parameter is an average feedback score across all feedback scores provided by each client associated with the respective wellness program.

In some embodiments, the plurality of coaching profiles, the plurality of wellness programs, the set of attributes assigned to the first client, or a combination thereof is processed using the plurality of computational models (e.g., using at least three computational models, at least 5 computational models, at least 15 computational models, etc.). In some embodiments, the plurality of coaching profiles, the plurality of wellness programs, the set of attributes assigned to the first client, and one or more corpus of communications is processed using the plurality of computational models. By processing the plurality of coaching profiles, the plurality of wellness programs, the set of attributes assigned to the first client, or the combination thereof, each respective computational model in the plurality of computational model produces a respective result that is a data set. In some embodiments, the data set of the respective result is associated with a wellness program in the plurality of wellness programs or a coach in the plurality of coaches. For instance, in some embodiments, the respective result includes a projected quality of the corresponding coach for the set of attributes assigned to the first client, a project quality of a mentor coach of the corresponding coach for the set of attributes assigned to the first client, a projected quality of one or more wellness programs associated with the corresponding coach for the set of attributes assigned to the first client, a projected accuracy and/or precision of a respective wellness program in the one or more wellness programs associated with the corresponding coach for the set of attributes assigned to the first client, a projected relevance quantity of the respective wellness program for the set of attributes assigned to the first client, a projected popularity of the corresponding coach for the set of attributes assigned to the first client, a projected achievement level of the corresponding coach for the set of attributes assigned to the first client, a projected return of investment associated with the corresponding for the set of attributes assigned to the first client, or a combination thereof. However, the present disclosure is not limited thereto. As another non-limiting example, in some embodiments, the respective result includes a first quantification of similarity of two or more user profiles (e.g., to determine a respective match for one or more wellness programs deemed complete by one or more clients deemed similar to the first client), a second quantification of similarity of at least two sets of attributes that includes the set of attributes assigned to the first client, such as two sets of goals (e.g., to determine a respective match for one or more wellness programs with one or more attribute similar to the at least to sets of attributes), a third quantification of similarity of at least two sets of attributes and at least two sets of wellness programs (e.g., to determine a best match for one or more wellness programs that satisfy each attribute in a set of attributes assigned to the client), a fourth quantification of similarity of least two texts in one or more corpus of communications (e.g., to determine one or more clusters of a plurality of wellness programs, a plurality of communication channels, a plurality of material, or a combination there associated with a corresponding subject matter), a fifth quantification of similarity of at least two wellness programs (e.g., to determine if a respective wellness program has an incorrect attribute in a set of attributes assigned to the first client), or a combination thereof. However, the present disclosure is not limited thereto. As yet another non-limiting example, in some embodiments, the respective result includes a measure of a number of attributes in the set of attributes assigned to the first client that appear in the respective wellness program (e.g., a first portion of keywords that is present in the title of the respective wellness program, a second portion of keywords that is present in the description of the respective wellness program, a third portion that is a number of medical condition attributes, etc.). In some embodiments, each respective result associated with a corresponding coach or a corresponding wellness program is stored in a corresponding historical data set associated with the corresponding coach or the corresponding wellness program, such as for future use with a second request for a wellness program from a second client.

In some embodiments, the systems and methods of the present disclosure produce a set of at least one coaching profile and at least one wellness program by collectively considering each respective result produced by each computational model in the plurality of computational models. For instance, in some embodiments, each respective result that is produced by each computational model is assigned a weight independently. As a non-limiting example, consider a first result having a first weight of about 5% and a second result having a weight of about 15%. However, the present disclosure is not limited thereto. For instance, as yet another non-limiting example, in some embodiments, the systems and methods of the present disclosure collectively consider the first portion of keywords that is present in the title of the respective wellness program at a first weight of about 40%, a second portion of keywords that is present in the description of the respective wellness program at a second weight of about 30%, a third portion that is a number of medical condition attributes of about 10%, a fourth portion that is a quantification of similarity between personal characteristic attributes of the first client and a respective client (e.g., personalized attributes) that have been deemed to compete a respective wellness program 210 and further deemed to have provided positive feedback (e.g., positive feedback for the respective wellness program 210 and/or the corresponding coach). In some embodiments, each respective result is collectively considered to determine a projected return of investment in accordance with a determination that the first client is deemed to have completed the respective wellness program 210. Accordingly, in some such embodiments, this collective considering produces a set of at least one coaching profile and at least one wellness program. Each coaching profile or wellness program in the set of the at least one coaching profile and the at least one wellness program is deemed a best, or optimal, match for the first client based on the collective consideration of the plurality of computational models.

In some embodiments, the systems and methods of the present disclosure include communicating the set of the at least one coaching profile and the at least one wellness program to a remote device. Accordingly, by communicating the set to the remote device, a subject associated with the free device is free to enroll the first trainee with a respective coaching profile or a respective wellness program 210 in this set. As such, in some embodiments, the remote device is associated with the first client. In alternative embodiments, the remote device is associated with a subject other than the first client. In some embodiments, the systems and methods of the present disclosure further generate a listing of the set of the at least one coaching profile and the at least one wellness program. In some such embodiments, the listing of the set is configured for display on the display of the remote device.

### REFERENCES CITED AND ALTERNATIVE EMBODIMENTS

All references cited herein are incorporated herein by reference in their entirety and for all purposes to the same extent as if each individual publication or patent or patent application was specifically and individually indicated to be incorporated by reference in its entirety for all purposes.

The present invention can be implemented as a computer program product that includes a computer program mechanism embedded in a non-transitory computer-readable storage medium. For instance, the computer program product could contain instructions for operating the user interfaces disclosed herein and described with respect to FIGS. 2A, 2B, 3, 5, 6, 7, 8, 9, 10, 11, and 12. These program modules can be stored on a CD-ROM, DVD, magnetic disk storage product, USB key, or any other non-transitory computer readable data or program storage product.

Many modifications and variations of this invention can be made without departing from its spirit and scope, as will be apparent to those skilled in the art. The specific embodiments described herein are offered by way of example only. The embodiments were chosen and described in order to best explain the principles of the invention and its practical applications, to thereby enable others skilled in the art to best utilize the invention and various embodiments with various modifications as are suited to the particular use contemplated. The invention is to be limited only by the terms of the appended claims, along with the full scope of equivalents to which such claims are entitled.

What is claimed is:

1. A method of matching a first client and a coach, the method comprising:
at a computer system comprising one or more processors and a memory storing at least one program for execution by the at least one processor, the at least one program comprising instructions for:
(A) receiving, in electronic form, a request for a wellness program, the request comprising a set of attributes assigned to the first client from a plurality of attributes;
(B) obtaining, in response to receiving the request, a plurality of coaching profiles, wherein each coaching profile in the plurality of coaching profiles: (i) is associated with a corresponding coach in a plurality of coaches, (ii) comprises a corresponding one or more wellness programs, each wellness program in the corresponding one or more wellness programs administrated, at least in part, by the corresponding coach, and (iii) a first corresponding data set associated with a corresponding first historical performance of the corresponding coach during a respective wellness program in the corresponding one or more wellness program;
(C) further obtaining, in response to receiving the request, a plurality of wellness programs, wherein each respective wellness program in the plurality of wellness program: (i) is associated with one or more corresponding coaches in the plurality of coaches, (ii) comprises one or more attributes improved by the respective wellness program, and (iii) a second corresponding data set associated with a second historical performance of the respective wellness program during the respective wellness program;
(D) processing, using a plurality of computational models, the plurality of coaching profiles, the plurality of wellness programs, and the set of attributes assigned to the first client, thereby producing a respective result for each computational model in the plurality of computational models, wherein each respective result is a data set associated with a wellness programs in the plurality of wellness programs or a coach in the plurality of coaches;
(E) collectively considering each respective result, thereby producing a set of at least one coaching profile and at least one wellness program; and
(F) communicating, in electronic format, to a remote device associated with the first client, the set of the at least one coaching profile and the at least one wellness program, thereby matching the first client and the coach.

2. The method of claim 1, wherein the set of attributes comprises one or more medical condition attributes associated with the first client, one or more lifestyle attributes associated with the first client, one or more temporal attributes associated with completing the respective wellness program, one or more geographic attributes associated with completing the respective wellness program, one or more accounting attributes associated with the respective wellness program, one or more physical attributes associated with the respective wellness program, one or more mental attributes associated with the respective wellness program, or a combination thereof.

3. The method of claim 2, wherein the set of attributes comprises the one or more accounting attributes, and wherein the one or more accounting attributes comprises a price of the respective wellness program, a schedule of the respective wellness program, a plurality of tasks associated with the respective wellness program, one or more quantitative goals associated with the respective wellness program, or a combination thereof.

4. The method of claim 1, wherein the corresponding first historical data set for a corresponding coach in the plurality of coaching profiles comprises a universal success rate for the one or more wellness programs associated with the corresponding coach, an individual success rate for each wellness program in the corresponding one or more wellness programs, a universal enrollment rate for the corresponding one or more wellness programs, an individual enrollment rate for each wellness program in the corresponding one or more wellness programs, an evaluation of communications corpus associated with the corresponding coach, an engagement rate for each wellness program in the corresponding one or more wellness programs, a frequency rate for each wellness program in the corresponding one or more wellness programs, a frequency rate for each wellness program in the corresponding one or more wellness programs, the data set associated with the corresponding one or more wellness programs, or a combination thereof.

5. The method of claim 4, wherein the corresponding first historical data set comprises the communications corpus associated with the corresponding coach, and wherein the communications corpus comprises a record of a corresponding plurality of messages for each communication channel in a plurality of communication channels associated with the corresponding coach.

6. The method of claim 5, wherein each communication channel in the plurality of communication channels facilitates an exchange of a plurality of messages between the corresponding coach and a respective subject in a plurality of subjects.

7. The method of claim 1, wherein a respective computational model in the plurality of computational models includes determining, for each respective sentiment in a plurality of sentiments, whether a corresponding sentiment analysis criterion is satisfied or not satisfied by taking a cosine similarly measure or dot product of one or more data elements in the corresponding coaching profile against each reference statement in a corresponding list of reference statements for the respective sentiment that are deemed to be attributive of a predetermined sentiment.

8. The method of claim 1, wherein the first client is associated with an enterprise that has vetted the coach.

9. The method of claim 1, wherein the at least one program further comprises instructions for (G) responsive to receiving a request for a match with the first coach in the set of the at least one coaching profile by the first client, matching the first coach with the first client, in the corresponding plurality of clients in accordance with an identification, by the plurality of computational models, that the first coach is a respective coach that best matches with a respective attribute of the first client.

10. The method of claim 1, wherein the data set associated with the one or more wellness programs comprises a weighted average of a subset of attributes in the set of attributes assigned to the first client.

11. The method of claim 10, wherein each respective attribute in the set of attributes comprises an independent weight.

12. The method of claim 1, wherein the data set associated with the one or more wellness program comprises a first return of investment of the first client and/or a second return on investment of a respective coach associated with a coaching profile in the set of coaching profiles.

13. The method of claim 1, wherein the communicating (F) further comprises generating, for display at the remote device, a listing of the set of the at least one coaching profile and the at least one wellness program.

14. The method of claim 1, wherein the at least one coaching profile and the at least one wellness program have a one to one relationship in the set.

15. The method of claim 1, wherein the remote device associated with a subject other than the first client.

16. The method of claim 1, wherein the respective result produced comprises a first similarity based on two or more user profiles, a second result based on at least two sets of attributes, a third result based on at least two sets of attributes and at least two sets of wellness programs a fourth result based on at least two texts in one or more corpus of communications, a fifth result based on at least two wellness programs.

17. The method of claim 1, wherein the first corresponding data set comprises a quality of the respective coach, a quality of a respective wellness program in the one or more wellness programs, a popularity of the respective coach, a popularity of the respective wellness program, or a combination thereof.

18. The method of claim 1, wherein the set of attributes comprises a first subset of attributes assigned to the first client by the plurality of computational models and a second subset of attributes assigned to the first client by a human subject.

19. A computer system comprising one or more processors and a memory storing at least one program for execution by the at least one processor, the at least one program comprising instructions for:
(A) receiving, in electronic form, a request for a wellness program, the request comprising a set of attributes assigned to the first client from a plurality of attributes;
(B) obtaining, in response to receiving the request, a plurality of coaching profiles, wherein each coaching profile in the plurality of coaching profiles: (i) is associated with a corresponding coach in a plurality of coaches, (ii) comprises a corresponding one or more wellness programs, each wellness program in the corresponding one or more wellness programs administrated, at least in part, by the corresponding coach, and (iii) a first corresponding data set associated with a corresponding first historical performance of the corresponding coach during a respective wellness program in the corresponding one or more wellness program;
(C) further obtaining, in response to receiving the request, a plurality of wellness programs, wherein each respective wellness program in the plurality of wellness program: (i) is associated with one or more corresponding coaches in the plurality of coaches, (ii) comprises one or more attributes improved by the respective wellness program, and (iii) a second corresponding data set associated with a second historical performance of the respective wellness program during the respective wellness program;
(D) processing, using a plurality of computational models, the plurality of coaching profiles, the plurality of wellness programs, and the set of attributes assigned to the first client, thereby producing a respective result for each computational model in the plurality of computational models, wherein each respective result is a data set associated with a wellness programs in the plurality of wellness programs or a coach in the plurality of coaches;
(E) collectively considering each respective result, thereby producing a set of at least one coaching profile and at least one wellness program; and
(F) communicating, in electronic format, to a remote device associated with the first client, the set of the at least one coaching profile and the at least one wellness program, thereby matching the first client and the coach.

20. A non-transitory computer readable storage medium stored on a computer system, the computer system comprising one or more processors and a memory storing at least one program for execution by the at least one processor, the at least one program comprising instructions for:
(A) receiving, in electronic form, a request for a wellness program, the request comprising a set of attributes assigned to the first client from a plurality of attributes;
(B) obtaining, in response to receiving the request, a plurality of coaching profiles, wherein each coaching profile in the plurality of coaching profiles: (i) is associated with a corresponding coach in a plurality of coaches, (ii) comprises a corresponding one or more wellness programs, each wellness program in the corresponding one or more wellness programs administrated, at least in part, by the corresponding coach, and (iii) a first corresponding data set associated with a corresponding first historical performance of the corresponding coach during a respective wellness program in the corresponding one or more wellness program;
(C) further obtaining, in response to receiving the request, a plurality of wellness programs, wherein each respective wellness program in the plurality of wellness program: (i) is associated with one or more corresponding coaches in the plurality of coaches, (ii) comprises one or more attributes improved by the respective wellness program, and (iii) a second corresponding data set associated with a second historical performance of the respective wellness program during the respective wellness program;
(D) processing, using a plurality of computational models, the plurality of coaching profiles, the plurality of wellness programs, and the set of attributes assigned to the first client, thereby producing a respective result for each computational model in the plurality of computational models, wherein each respective result is a data set associated with a wellness programs in the plurality of wellness programs or a coach in the plurality of coaches;
(E) collectively considering each respective result, thereby producing a set of at least one coaching profile and at least one wellness program; and
(F) communicating, in electronic format, to a remote device associated with the first client, the set of the at least one coaching profile and the at least one wellness program, thereby matching the first client and the coach.
